(19) United States
(12) Patent Application Publication
(10) Pub. No.: US 2024/0002494 A1
(43) Pub. Date: Jan. 4, 2024

(54) Safe and Effective Method of Treating Ulcerative Colitis with Anti-IL12/IL23 Antibody

(71) Applicant: Janssen Biotech, Inc., Horsham, PA (US)

(72) Inventors: Jewel Johanns, Devon, PA (US); Katherine Li, Wallingford, PA (US); Colleen Marano, Malvern, PA (US); Richard Strauss, Doylestown, PA (US); Hongyan Zhang, Malvern, PA (US); Christopher O'Brien, Lafayette Hill, PA (US); Omoniyi Adedokun, Phoenixville, PA (US); Kimberly Shields-Tuttle, West Chester, PA (US)

(21) Appl. No.: 18/210,885

(22) Filed: Jun. 16, 2023

Related U.S. Application Data
(62) Division of application No. 17/175,129, filed on Feb. 12, 2021, now abandoned.
(60) Provisional application No. 62/976,582, filed on Feb. 14, 2020.

(51) Int. Cl. C07K 16/24; A61P 1/04

(52) U.S. Cl. CPC C07K 16/244; A61P 1/04; A61K 2039/545

(57) ABSTRACT

Described are methods and compositions for clinical proven safe and effective treatment of ulcerative colitis, particularly moderately to severely active ulcerative colitis in patients who have had an inadequate response to or are intolerant of a conventional or existing therapy by intravenous and/or subcutaneous administration of an anti-IL-12/IL-23p40 antibody.

### CROSS-REFERENCE TO RELATED APPLICATIONS

This application is a divisional of U.S. patent application Ser. No. 17/175,129, filed 12 Feb. 2021, currently pending, which claims priority to U.S. Provisional Application No. 62/976,582, filed 14 Feb. 2020. The entire contents of each of the aforesaid applications are incorporated herein by reference in their entireties.

### REFERENCE TO SEQUENCE LISTING SUBMITTED ELECTRONICALLY

This application contains a sequence listing, which is submitted electronically via EFS-Web as an ASCII formatted sequence listing with a file name “JBI6165USDIV1Sequence Listing.xml” creation date of 16 Jun. 2023, and having a size of 15 kilobytes. The sequence listing submitted via Patent Center is part of the specification and is herein incorporated by reference in its entirety.

### FIELD OF THE INVENTION

The invention relates to methods of providing a clinically proven safe and clinically proven effective treatment of ulcerative colitis, particularly moderately to severely active ulcerative colitis in patients who have had an inadequate response to or are intolerant of a conventional or existing therapy by intravenous and/or subcutaneous administration of an anti-IL-12/IL-23p40 antibody.

### BACKGROUND OF THE INVENTION

Inflammatory bowel diseases (IBDs), including ulcerative colitis (UC), are chronic relapsing disorders characterized by destructive inflammation and epithelial injury in the gastrointestinal (GI) tract (Baumgart and Sandborn, J Clin Invest. 98:1010-1020 (1996); Danese and Fiocchi, N Engl J Med. 365:1715-1725 (2011)). The incidence of UC in the United States is estimated to be between 9 and 12 per 100,000 persons with a prevalence of 205 to 240 per 100,000 persons (Tally et al., Am J Gastroenterol. 106 Suppl 1:S2-S25 (2011)). The estimate of the prevalence of UC in Europe is approximately 1 million people (Loftus, Gastroenterology. 126(6):1504-1517 (2004); Loftus, Gastoenterol Clin N Am. 31:1-20 (2002)). The etiology of UC is unknown. However, abnormal immune responses to contents in the gut, including intestinal microbes, are thought to drive disease in genetically predisposed individuals (Geremia et al., Autoimmun Rev. 13:3-10 (2014)). Dysregulated innate and adaptive immune pathways contribute to aberrant intestinal inflammation in IBD, and cytokines, including interleukin (IL)-12, interferon-gamma (IFNγ), and IL-23 have been implicated in the pathogenesis of UC (Geremia et al., Autoimmune Rev. 2014; 13:3-10; Neurath, Nat Rev Immunol. 14(5):329-42 (2014)).

The involvement of the IL-12/23 pathway in the pathogenesis of IBD is well established, and an important role for IL-12/IL-23 pathway in intestinal inflammation has been elucidated in colitis (Ahern et al., Immunity. 33(2):279-288 (2010); Investigator's Brochure: STELARA® (ustekinumab), edition 18. Janssen Research & Development, LLC (2017); Uhlig et al., Immunity. 25:309 318 (2006); Yen et al., J Clin Invest. 116(5):1310-1316 (2006)). Early studies showed that treatment with anti-IFNγ (Berg et al., J Clin Invest. 98:1010-1020 (1996); Davidson et al., J Immunol. 161:3143-3149 (1998)) or anti-IL-12p40 monoclonal antibodies (mAb) prevented disease in experimental colitis models, suggesting an important role for type 1 T helper (Th-1) cells in promoting intestinal inflammation (Neurath et al., J Exp Med. 182(5):1281-1290 (1995)). Genome-wide association studies have implicated several genetic loci in humans in the IL-12/23 pathway that are associated with increased susceptibility to UC, including IL-23R and IL-12B (Anderson et al., Nat Genet. 43(3):246-252 (2011); Brant et al., Clin Gastroenterol Hepatol. 11(1):22-26 (2013)). Subjects with active UC were shown to have significantly more IL-23, IL-22, IL-22R1 and p-STAT3-positive cells than subjects with inactive UC and normal controls (Yu et al., World J Gastroenterol. 19(17):2638-2649 (2013)).

Biologic therapies currently approved for the treatment of UC are either tumor necrosis factor (TNF) or integrin inhibitors (Colombel et al., Gastroenterology. 132:52-65 (2007); Hanauer et al., Lancet. 359:1541-1549 (2002); Sandborn et al., N Engl J Med. 369:711-721 (2013); Sandborn et al., Gastroenterology. 142:257-265 (2012)). However, only 1 therapy of all currently approved treatments, vedolizumab, has demonstrated efficacy in subjects who have had an inadequate response to (i.e., primary nonresponse or secondary loss of response) or are intolerant of anti-TNFs (Feagan et al., N Engl J Med. 369:699 710 (2013)). Anti-TNFs have safety risks associated with immunosuppression and not all subjects adequately respond to such therapy. Furthermore, as was observed with the anti-TNFs, inadequate response, and intolerance has been identified in subjects receiving vedolizumab for the treatment of their UC. Therefore, there remains an unmet need for novel therapies with alternative mechanisms of action.

When tested, biologic therapies that are currently approved for the treatment of UC have also demonstrated efficacy in Crohn's disease (Sandborn et al., Gastroenterology. 135(4):1130-1141 (2008)). Multiple lines of evidence suggest that inflammatory bowel disease (UC and Crohn's disease) is mediated by Th1 or Th17 cells with strong contribution from the proinflammatory cytokines, IL-12, and IL-23. Ustekinumab (STELARA®) is a fully human immunoglobulin G1 mAb to human IL-12/23p40 that prevents IL-12 and IL-23 bioactivity by inhibiting their interaction with their cell surface IL-12Rβ1 receptor protein (Investigator's Brochure: STELARA® (ustekinumab), edition 18. Janssen Research & Development, LLC (2017)). Through this mechanism of action, ustekinumab effectively neutralizes IL-12 (Th1)- and IL-23 (Th17)-mediated cellular responses. Ustekinumab has received marketing approval globally, including countries in North America, Europe, South America, and the Asia-Pacific region, for the treatment of adult subjects with moderately to severely active Crohn's disease (the first approval for Crohn's disease was received on 11 Nov. 2016), moderate to severe plaque psoriasis, or active psoriatic arthritis, as well as for pediatric subjects (12 to 17 years old) with moderate to severe plaque psoriasis.

The efficacy and safety of intravenous (IV) ustekinumab as induction therapy in Crohn's disease have been evaluated in clinical studies CRD3001 and CRD3002. In study CRD3001, subjects with demonstrated prior failure or intolerance to one or more TNF antagonists were evaluated, and in CRD3002 subjects with history of inadequate response to or intolerance of corticosteroids or immunomodulators, but without a history of an inadequate response or intolerance to TNF antagonists were evaluated. In these studies, two IV doses were evaluated: a 130 mg IV fixed dose (˜2 mg/kg on a mg/kg basis) was chosen for the low-dose group, while body-weight range based doses approximating ˜6 mg/kg IV (weight ≤55 kg: ustekinumab 260 mg; weight >55 and ≤85 kg: ustekinumab 390 mg; weight >85 kg: ustekinumab: 520 mg) were chosen as the high-dose group. In both studies, ustekinumab demonstrated clinically significant efficacy compared with placebo and was well-tolerated with a favorable safety profile.

Prior to the present invention, no studies had been conducted with ustekinumab for UC. there is a need in the art for improved methods of treating UC, particularly moderately to severely active UC, in subjects who had previously failed or were intolerant of a biologic therapy or other conventional therapy, or subjects who had demonstrated corticosteroid dependence.

### BRIEF SUMMARY OF THE INVENTION

The present application relates to clinically proven safe and clinically proven effective methods and compositions for treatment of moderately to severely active ulcerative colitis (UC), particularly in subjects who have had an inadequate response to or are intolerant of a conventional or existing therapy, by administration of an anti-IL-12/IL-23p40 antibody to subjects, thereby addressing a clear unmet medical need in this subject population.

In one general aspect, the application relates to a clinically proven safe and clinically proven effective method of treating moderately to severely active ulcerative colitis (UC) in a subject in need thereof, comprising administering to the subject a pharmaceutical composition comprising a safe and effective amount of an anti-IL-12/IL-23p40 antibody, wherein the antibody comprises a heavy chain variable region and a light chain variable region, the heavy chain variable region comprising: a complementarity determining region heavy chain 1 (CDRH1) amino acid sequence of SEQ ID NO:1; a CDRH2 amino acid sequence of SEQ ID NO:2; and a CDRH3 amino acid sequence of SEQ ID NO:3; and the light chain variable region comprising: a complementarity determining region light chain 1 (CDRL1) amino acid sequence of SEQ ID NO:4; a CDRL2 amino acid sequence of SEQ ID NO:5; and a CDRL3 amino acid sequence of SEQ ID NO:6.

In certain embodiments, the anti-IL-12 and/or anti-IL-23 antibody is administered intravenously to the subject, preferably at week 0, at a dosage of about 6.0 mg/kg body weight of the subject or 130 mg per administration.

In certain embodiments, the anti-IL-12 and/or anti-IL-23 antibody is administered intravenously or subcutaneously to the subject, preferably at week 8, at a dosage of about 6.0 mg/kg body weight of the subject or 90 mg per administration, respectively.

Preferably, the subject treated by methods according to embodiments of the application has had an inadequate response to or are intolerant of a conventional or existing therapy. In some embodiments, the subject had previously failed or were intolerant of a biologic therapy, such as an anti-TNF and/or vedolizumab. In some embodiments, the subject had previously failed or were intolerant of a non-biologic therapy, such as a treatment with corticosteroids, azathioprine (AZA), and/or 6 mercaptopurine (6 MP). In some embodiments, the subject had demonstrated corticosteroid dependence.

In another general aspect, the application relates to a clinically proven safe and clinically proven effective method of treating moderately to severely active ulcerative colitis (UC) in a subject in need thereof, comprising:
intravenously administering to the subject a pharmaceutical composition comprising an anti-IL-12/IL-23p40 antibody at a dosage of about 6.0 mg/kg body weight of the subject or 130 mg of the antibody per administration at week 0 of the treatment, and
subcutaneously administering to the subject a pharmaceutical composition comprising the anti-IL-12/IL-23p40 antibody at a dosage of 90 mg of the antibody per administration at week 8 of the treatment,
wherein the antibody comprises a heavy chain variable region and a light chain variable region, the heavy chain variable region comprising: a complementarity determining region heavy chain 1 (CDRH1) amino acid sequence of SEQ ID NO:1; a CDRH2 amino acid sequence of SEQ ID NO:2; and a CDRH3 amino acid sequence of SEQ ID NO:3; and the light chain variable region comprising: a complementarity determining region light chain 1 (CDRL1) amino acid sequence of SEQ ID NO:4; a CDRL2 amino acid sequence of SEQ ID NO:5; and a CDRL3 amino acid sequence of SEQ ID NO:6; and
wherein the subject had previously failed or were intolerant of at least one therapy selected from the group consisting of: an anti-TNF, vedolizumab, corticosteroids, azathioprine (AZA), and 6 mercaptopurine (6 MP), or the subject had demonstrated corticosteroid dependence

In certain embodiments, methods of the present application comprise intravenously (IV) and/or subcutaneously (SC) administering to the subject a pharmaceutical composition comprising an anti-IL-12 and/or anti-IL-23 antibody or antigen binding fragment comprising: (i) a heavy chain variable domain amino acid sequence of SEQ ID NO:7; and (ii) a light chain variable domain amino acid sequence of SEQ ID NO:8.

In certain embodiments, methods of the present application comprise intravenously (IV) and/or subcutaneously (SC) administering to the subject a pharmaceutical composition comprising the anti-IL-12/23p40 antibody ustekinumab, which comprises: (i) a heavy chain amino acid sequence of SEQ ID NO:10; and (ii) a light chain amino acid sequence of SEQ ID NO:11.

In certain embodiments, the IV dose at week 0 is about 6.0 mg/kg. For example, the IV dose is 260 mg for subjects with body weight ≥35 kg and ≤55 kg, 390 mg for subjects with body weight >55 kg and ≥85 kg, and 520 mg for subjects with body weight >85 kg.

In certain embodiments, the subject is a responder to a treatment of a method according to an embodiment of the application, measured preferably 92 weeks after initial treatment and after maintenance doses have been received, and is identified as having at least one of: (1) a clinical remission based on at least one of the global submissions and the US submissions; (2) an endoscopic healing; (3) a clinical response; (4) a change from baseline in Inflammatory Bowel Disease Questionnaire (IBDQ) score; (5) a mucosal healing; (6) a decrease from baseline in Mayo score; and (7) a normalization of one or more biomarkers selected from the group consisting of C-reactive protein, fecal lactoferrin and fecal calprotectin. Preferably, at least one of (1) to (7) above is identified from the subject by week 16, more preferably by week 8 or week 4, and most preferably by week 2 of the treatment.

In certain embodiments, the present invention provides a clinically proven safe and clinically proven effective method of treating moderately to severely active UC in a subject, wherein the subject is a responder to the treatment with the antibody and is identified as having a statistically significant improvement in disease activity as determined by endoscopic healing with a Mayo endoscopy subscore of 0 or 1 by week 8 of treatment with the antibody. In other embodiments, the present invention provides a clinically proven safe and clinically proven effective method of treating moderately to severely active UC in a subject, wherein the subject is a responder to the treatment with the antibody and is identified as having a statistically significant improvement in disease activity as determined by an Ulcerative Colitis Endoscopic Index of Severity (UCEIS) score of ≤4 by week 8 of treatment with the antibody.

In certain embodiments, the subject is in clinical response as determined by a decrease from baseline in the Mayo score by ≥30% and ≥3 points and a decrease from baseline in the rectal bleeding subscore ≥1 points or a rectal bleeding subscore of 0 or 1 by week 8 of treatment with the antibody.

In other embodiments, a maintenance dose of the anti-IL-12/IL-23p40 antibody is administered every 8 weeks after the treatment at week 8 or every 12 weeks after the treatment at week 8 and clinical response is maintained by the subject for at least 44 weeks.

In certain embodiments, the present invention provides a clinically proven safe and clinically proven effective method of treating moderately to severely active UC in a subject, wherein a subject identified as a non-responder to an initial treatment is administered a second treatment, preferably with an administration route different from the initial treatment. For example, a subject identified as a non-responder to an initial treatment with an IV administration of an antibody or antibody binding fragment can be treated with a subsequent subcutaneous administration of the antibody or antibody binding fragment according to embodiments of the invention.

In certain embodiments, the present application provides for a method of treating moderately to severely active UC in a subject, wherein an anti-IL-12 and/or anti-IL-23 antibody for use with IV administration is in a pharmaceutical composition comprising a solution comprising 10 mM L-histidine, 8.5% (w/v) sucrose, 0.04% (w/v) polysorbate 80, 0.4 mg/mL L methionine, and 20 μg/mL EDTA disodium salt, dehydrate, at pH 6.0.

In certain embodiments, the present application provides for a clinically proven safe and clinically proven effective method of treating moderately to severely active UC in a subject, wherein an anti-IL-12 and/or anti-IL-23 antibody for use with subcutaneous administration is in a pharmaceutical composition comprising a solution comprising 6.7 mM L-histidine, 7.6% (w/v) sucrose, 0.004% (w/v) polysorbate 80, at pH 6.0.

In certain embodiments, the present application provides a method further comprising administering to the subject one or more additional drugs used to treat UC. In a preferred embodiment, the additional drug is selected from the group consisting of: oral 5-aminosalicylate (5-ASA) compounds, oral corticosteroids, immunomodulators, 6-mercaptopurine (6-MP), azathioprine (AZA), or methotrexate (MTX).

Other aspects of the application include pharmaceutical compositions comprising an anti-IL-12 and/or anti-IL-23 antibody for use in a clinically proven safe and clinically proven effective method of treating moderately to severely active UC in a subject, as well as methods of preparing the compositions and kits comprising the pharmaceutical compositions.

In certain embodiments, a kit useful for a method of the invention comprises at least one of a pharmaceutical composition for intravenous administration of the invention and pharmaceutical composition for subcutaneous administration of the invention. In other embodiments, the kit comprises both a pharmaceutical composition for intravenous administration and a pharmaceutical composition for subcutaneous administration of the invention.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

FIG. 1 shows a diagrammatic representation of the study design of the induction and maintenance studies of the Phase 3 Program Design. Abbreviations: W8=Week 8; W16=Week 16; LTE=Long-term Extension.

FIG. 2 shows the maintenance study of the Phase 3 Program Design.

FIG. 3 shows the disposition of subjects by Maintenance Week 0 Treatment in CNTO1275UC03001 through Week 96 of randomized subjects.

FIG. 4 shows the disposition of subjects by Maintenance Week 0 Treatment in CNTO1275USO3001 through week 96 of nonrandomized subjects.

FIG. 5 shows a proportion of subjects in symptomatic remission over time through Week 92 or up to the time of Dose Adjustment of randomized subjects in maintenance study who were treated in the LTE (CNTO1275USO3001).

FIG. 6 shows the mean daily prednisone-equivalent Corticosteroid dose (mg/day) over time from Week 0 through Week 92 among subjects receiving Corticosteroids other than budesonide and beclomethasone dipropionate at the maintenance baseline.

FIG. 7 shows the number of subjects in symptomatic remission over time through week 92 where the dose adjustment is not considered as treatment failure of randomized subjects in maintenance who were treated in the long-term extension.

FIG. 8 shows the number of subjects in symptomatic remission over time through Week 92 where all subjects were randomized at week 0 of maintenance and the dose adjustment is not considered as treatment failure.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, “subject” means any animal, preferably a mammal, most preferably a human, whom will be or has been treated by a method according to an embodiment of the invention. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, humans, etc., more preferably a human.

As used herein, the term “in combination”, in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

As used herein, an “anti-IL-12 antibody,” “anti-IL-23 antibody,” “anti-IL-12/23p40 antibody,” or “IL-12/23p40 antibody,” refers to a monoclonal antibody (mAb) or antigen binding fragment thereof, that binds the 40 kDa (p40) subunit shared by the cytokines interleukin-12 and interleukin-23 (IL-12/23p40). The antibody can affect at least one of IL-12/23 activity or function, such as but not limited to, RNA, DNA or protein synthesis, IL-12/23 release, IL-12/23 receptor signaling, membrane IL-12/23 cleavage, IL-12/23 activity, IL-12/23 production and/or synthesis.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian IL-12/23. For example, antibody fragments capable of binding to IL-12/23 or portions thereof, including, but not limited to, Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the C_{H}1 domain and/or hinge region of the heavy chain. The various portions of antibodies can be joined together chemically by conventional techniques, or can be prepared as a contiguous protein using genetic engineering techniques.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A “human antibody” can also be an antibody that is derived from or closely matches human germline immunoglobulin sequences. Human antibodies can include amino acid residues not encoded by germline immunoglobulin sequences (e.g., mutations introduced by random or site-specific mutagenesis in vitro or by somatic mutation in vivo). Often, this means that the human antibody is substantially non-immunogenic in humans. Human antibodies have been classified into groupings based on their amino acid sequence similarities. Accordingly, using a sequence similarity search, an antibody with a similar linear sequence can be chosen as a template to create a human antibody. Similarly, antibodies designated primate (monkey, baboon, chimpanzee, etc.), rodent (mouse, rat, rabbit, guinea pig, hamster, and the like) and other mammals designate such species, sub-genus, genus, sub-family, and family specific antibodies. Further, chimeric antibodies can include any combination of the above. Such changes or variations optionally and preferably retain or reduce the immunogenicity in humans or other species relative to non-modified antibodies. Thus, a human antibody is distinct from a chimeric or humanized antibody.

It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

Anti-IL-12/23p40 antibodies (also termed IL-12/23p40 antibodies) (or antibodies to IL-23) useful in the methods and compositions of the present invention can optionally be characterized by high affinity binding to IL-12/23p40, optionally and preferably, having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat subjects for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the subjects treated and/or raising low titres in the subject treated (less than about 300, preferably less than about 100 measured with a double antigen enzyme immunoassay) (Elliott et al., Lancet 344:1125-1127 (1994), entirely incorporated herein by reference). “Low immunogenicity” can also be defined as the incidence of titrable levels of antibodies to the anti-IL-12 antibody in subjects treated with anti-IL-12 antibody as occurring in less than 25% of subjects treated, preferably, in less than 10% of subjects treated with the recommended dose for the recommended course of therapy during the treatment period.

The terms “clinically proven efficacy” and “clinically proven effective” as used herein in the context of a dose, dosage regimen, treatment or method refer to the effectiveness of a particular dose, dosage or treatment regimen. Efficacy can be measured based on change in the course of the disease in response to an agent of the present invention. For example, an anti-IL12/23p40 of the present invention (e.g., ustekinumab) is administered to a subject in an amount and for a time sufficient to induce an improvement, preferably a sustained improvement, in at least one indicator that reflects the severity of the disorder that is being treated. Various indicators that reflect the extent of the subject's illness, disease or condition can be assessed for determining whether the amount and time of the treatment is sufficient. Such indicators include, for example, clinically recognized indicators of disease severity, symptoms, or manifestations of the disorder in question. The degree of improvement generally is determined by a physician, who can make this determination based on signs, symptoms, biopsies, or other test results, and who can also employ questionnaires that are administered to the subject, such as quality-of-life questionnaires developed for a given disease. For example, an anti-IL12/23p40 or anti-IL23 antibody of the present invention can be administered to achieve an improvement in a subject's condition related to ulcerative colitis.

Improvement can be indicated by an improvement in an index of disease activity, by amelioration of clinical symptoms or by any other measure of disease activity. Once such index of disease is the ulcerative colitis Mayo score. The Mayo score is an established, validated disease activity index for mild, moderate, and severe ulcerative colitis (UC) that is calculated as the sum of the 4 subscores of stool frequency, rectal bleeding, findings of endoscopy, and physician's global assessment (PGA), and ranges from 0-12. A score of 3 to 5 points indicates mildly active disease, a score of 6 to 10 points indicates moderately active disease, and a score of 11 to 12 points indicates severe disease. The partial Mayo score, which is the Mayo score without the endoscopy subscore, is calculated as the sum of stool frequency, rectal bleeding, and physician's global assessment subscores, and ranges from 0 to 9. The modified Mayo score, which is the Mayo score without the PGA subscore, is calculated as the sum of the stool frequency, rectal bleeding, and endoscopy subscores, and ranges from 0 to 9. Other disease activity indexes for UC include for example, Ulcerative Colitis Endoscopic Index of Severity (UCEIS) score and the Bristol Stool Form Scale (BSFS) score. The UCEIS score provides an overall assessment of endoscopic severity of UC, based on mucosal vascular pattern, bleeding, and ulceration (Travis et al., Gut. 61:535-542 (2012)). The score ranges from 3 to 11 with a higher score indicating more severe disease by endoscopy. The BSFS score is used to classify the form (or consistency) of human feces into 7 categories (Lewis and Heaton, Scand J Gastroenterol. 32(9):920-924 (1997)).

The term “clinical response” as used herein as it relates to a subject's response to drug administration, refers to a decrease from induction baseline in the Mayo score by ≥30% and ≥3 points, with either a decrease from baseline in the rectal bleeding subscore ≥1 or a rectal bleeding subscore of 0 or 1.

The term “clinically proven safe,” as it relates to a dose, dosage regimen, treatment or method with anti-IL-12/IL-23p40 antibody of the present invention (e.g., ustekinumab), refers to a favorable risk:benefit ratio with an acceptable frequency and/or acceptable severity of treatment-emergent adverse events (referred to as AEs or TEAEs) compared to the standard of care or to another comparator. As used herein, “adverse event,” “treatment-emergent adverse event,” and “adverse reaction” mean any harm, unfavorable, unintended or undesired sign or outcome associated with or caused by administration of a pharmaceutical composition or therapeutic. It is an untoward medical occurrence in a subject administered a medicinal product. However, abnormal values or observations are not reported as adverse events unless considered clinically significant by the investigator. As used herein, when referring to an adverse event, “clinically apparent” means clinically significant as determined by a medical doctor or an investigator using standard acceptable to those of ordinary skill in the art. When the harm or undesired outcome of adverse events reaches such a level of severity, a regulatory agency can deem the pharmaceutical composition or therapeutic unacceptable for the proposed use. In particular, “safe” as it relates to a dose, dosage regimen or treatment with an anti-IL12/23p40 or anti-IL23 antibody of the present invention refers to with an acceptable frequency and/or acceptable severity of adverse events associated with administration of the antibody if attribution is considered to be possible, probable, or very likely due to the use of the anti-IL12/23p40 or anti-IL23 antibody.

As used herein, unless otherwise noted, the term “clinically proven” (used independently or to modify the terms “safe” and/or “effective”) shall mean that it has been proven by a clinical trial wherein the clinical trial has met the approval standards of U.S. Food and Drug Administration, EMEA or a corresponding national regulatory agency. For example, the clinical study may be an adequately sized, randomized, double-blinded study used to clinically prove the effects of the drug.

As used herein, a dosage amount of an anti-IL-12/IL-23p40 antibody in “mg/kg” refers to the amount of the anti-IL-12/IL-23p40 antibody in milligrams per kilogram of the body weight of a subject to be administered with the antibody.

Antibodies of the Present Invention—Production and Generation

At least one anti-IL-12/23p40 (or anti-IL-23) used in the method of the present invention can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2nd Edition, Cold Spring Harbor, NY (1989); Harlow and Lane, antibodies, a Laboratory Manual, Cold Spring Harbor, N Y (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001), each entirely incorporated herein by reference.

Human antibodies that are specific for human IL-12/23p40 or IL-23 proteins or fragments thereof can be raised against an appropriate immunogenic antigen, such as an isolated IL-12/23p40 protein, IL-23 protein and/or a portion thereof (including synthetic molecules, such as synthetic peptides). Other specific or general mammalian antibodies can be similarly raised. Preparation of immunogenic antigens, and monoclonal antibody production can be performed using any suitable technique in view of the present disclosure.

In one approach, a hybridoma is produced by fusing a suitable immortal cell line (e.g., a myeloma cell line, such as, but not limited to, Sp2/0, Sp2/0-AG14, NSO, NS1, NS2, AE-1, L.5, L243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMALWA, NEURO 2A, or the like, or heteromylomas, fusion products thereof, or any cell or fusion cell derived therefrom, or any other suitable cell line as known in the art) (see, e.g., www.atcc.org, www.lifetech.com, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Antibody producing cells can also be obtained from the peripheral blood or, preferably, the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; BioInvent, Lund, Sweden; Dyax Corp., Enzon, Affymax/Biosite; Xoma, Berkeley, CA; Ixsys. See, e.g., EP 368,684, PCT/GB91/01134; PCT/GB92/01755; PCT/GB92/002240; PCT/GB92/00883; PCT/GB93/00605; U.S. Ser. No. 08/350,260 (5/12/94); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); WO96/13583, WO97/08320 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590 689 (Ixsys, predecessor of Applied Molecular Evolution (AME), each entirely incorporated herein by reference)) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (Can 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, MA; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source that is non-human, e.g., but not limited to, mouse, rat, rabbit, non-human primate or other mammal. These non-human amino acid residues are replaced by residues often referred to as “import” residues, which are typically taken from an “import” variable, constant or other domain of a known human sequence.

Known human Ig sequences are disclosed, e.g., www. ncbi.nlm.nih.gov/entrez/query.fcgi; www.ncbi.nih.gov/igblast; www.atcc.org/phage/hdb.html; www.mrc-cpe.cam.ac.uk/ALIGNMENTS.php; www.kabatdatabase.com/top.html; ftp.ncbi.nih.gov/repository/kabat; www.sciquest.com; www.abcam.com; www. antibodyresource.com/onlinecomp.html; www.public.iastate.edu/-pedro/research_tools.html; www.whfreeman.com/immunology/CH05/kuby05.htm; www. hhmi.org/grants/lectures/1996/vlab; www.path.cam.ac.uk/-mrc7/mikeimages.html; mcb.harvard.edu/BioLinks/Immunology.html; www.immunologylink.com; pathbox.wustl.edu/-hcenter/index.html; www.appliedbiosystems.com; www. nal.usda.gov/awic/pubs/antibody; www.m.ehime-u.ac.jp/-yasuhito/Elisa.html; www. biodesign.com; www.cancerresearchuk.org; www.biotech.ufl.edu; www.isac-net.org; baserv.uci.kun.nl/-jraats/links1.html; www.recab.uni-hd.de/immuno.bme.nwu.edu; www.mrc-cpe.cam.ac.uk; www.ibt.unam.mx/vir/V_mice.html; www.bioinf.org.uk/abs; antibody.bath.ac.uk; www.unizh.ch; www.cryst.bbk.ac.uk/-ubcg07s; www. nimr.mrc.ac.uk/CC/ccaewg/ccaewg.html; www. path.cam.ac.uk/-mrc7/humanisation/TAHHP.html; www. ibt.unam.mx/vir/structure/stat_aim.html; www.biosci.missouri.edu/smithgp/index.html; www. jerini.de; Kabat et al., Sequences of Proteins of Immunological Interest, U.S. Dept. Health (1983), each entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing antigen binding. Accordingly, part or all of the non-human or human CDR sequences are maintained while the non-human sequences of the variable and constant regions can be replaced with human or other amino acids.

Antibodies can also optionally be humanized or human antibodies engineered with retention of high affinity for the antigen and other favorable biological properties. To achieve this goal, humanized (or human) antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

In addition, the human anti-IL-12/23p40 (or anti-IL-23) specific antibody used in the method of the present invention can comprise a human germline light chain framework. In particular embodiments, the light chain germline sequence is selected from human VK sequences including, but not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8. In certain embodiments, this light chain human germline framework is selected from V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6.

In other embodiments, the human anti-IL-12/23p40 (or anti-IL-23) specific antibody used in the method of the present invention can comprise a human germline heavy chain framework. In particular embodiments, this heavy chain human germline framework is selected from VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81.

In particular embodiments, the light chain variable region and/or heavy chain variable region comprises a framework region or at least a portion of a framework region (e.g., containing 2 or 3 subregions, such as FR2 and FR3). In certain embodiments, at least FRL1, FRL2, FRL3, or FRL4 is fully human. In other embodiments, at least FRH1, FRH2, FRH3, or FRH4 is fully human. In some embodiments, at least FRL1, FRL2, FRL3, or FRL4 is a germline sequence (e.g., human germline) or comprises human consensus sequences for the particular framework (readily available at the sources of known human Ig sequences described above). In other embodiments, at least FRH1, FRH2, FRH3, or FRH4 is a germline sequence (e.g., human germline) or comprises human consensus sequences for the particular framework. In preferred embodiments, the framework region is a fully human framework region.

Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, PCT/: US98/16280, US96/18978, US91/09630, US91/05939, US94/01234, GB89/01334, GB91/01134, GB92/01755; WO90/14443, WO90/14424, WO90/14430, EP 229246, each entirely incorporated herein by reference, included references cited therein.

In certain embodiments, the antibody comprises an altered (e.g., mutated) Fc region. For example, in some embodiments, the Fc region has been altered to reduce or enhance the effector functions of the antibody. In some embodiments, the Fc region is an isotype selected from IgM, IgA, IgG, IgE, or other isotype. Alternatively, or additionally, it can be useful to combine amino acid modifications with one or more further amino acid modifications that alter C1q binding and/or the complement dependent cytotoxicity function of the Fc region of an IL-23 binding molecule. The starting polypeptide of particular interest can be one that binds to C1q and displays complement dependent cytotoxicity (CDC). Polypeptides with pre-existing C1q binding activity, optionally further having the ability to mediate CDC can be modified such that one or both of these activities are enhanced. Amino acid modifications that alter C1q and/or modify its complement dependent cytotoxicity function are described, for example, in WO0042072, which is hereby incorporated by reference.

As disclosed above, one can design an Fc region of the human anti-IL-12/23p40 (or anti-IL-23) specific antibody of the present invention with altered effector function, e.g., by modifying C1q binding and/or FcγR binding and thereby changing complement dependent cytotoxicity (CDC) activity and/or antibody-dependent cell-mediated cytotoxicity (ADCC) activity. “Effector functions” are responsible for activating or diminishing a biological activity (e.g., in a subject). Examples of effector functions include, but are not limited to: C1q binding; CDC; Fc receptor binding; ADCC; phagocytosis; down regulation of cell surface receptors (e.g., B cell receptor; BCR), etc. Such effector functions can require the Fc region to be combined with a binding domain (e.g., an antibody variable domain) and can be assessed using various assays (e.g., Fc binding assays, ADCC assays, CDC assays, etc.).

For example, one can generate a variant Fc region of the human anti-IL-12/23p40 (or anti-IL-23) antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities can be increased, and, optionally, also the other activity reduced (e.g., to generate an Fc region variant with improved ADCC activity, but reduced CDC activity and vice versa).

Fc mutations can also be introduced in engineer to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, J. Biol. Chem. 276:6591-6604).

Another type of amino acid substitution serves to alter the glycosylation pattern of the Fc region of the human anti-IL-12/23p40 (or anti-IL-23) specific antibody. Glycosylation of an Fc region is typically either N-linked or O-linked. N-linked refers to the attachment of the carbohydrate moiety to the side chain of an asparagine residue. O-linked glycosylation refers to the attachment of one of the sugars N-aceylgalactosamine, galactose, or xylose to a hydroxyamino acid, most commonly serine or threonine, although 5-hydroxyproline or 5-hydroxylysine can also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern can be altered, for example, by deleting one or more glycosylation site(s) found in the polypeptide, and/or adding one or more glycosylation sites that are not present in the polypeptide. Addition of glycosylation sites to the Fc region of a human IL-23 specific antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration can also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the human anti-IL-12/23p40 (or anti-IL-23) specific antibody of the present invention is expressed in cells that express beta (1,4)-N-acetylglucosaminyltransferase III (GnT III), such that GnT III adds GlcNAc to the human anti-IL-12/23p40 (or anti-IL-23) antibody. Methods for producing antibodies in such a fashion are provided in WO/9954342, WO/03011878, patent publication 20030003097A1, and Umana et al., Nature Biotechnology, 17:176-180, February 1999; all of which are herein specifically incorporated by reference in their entireties.

The human anti-IL-12/23p40 (or anti-IL-23) antibody can also be optionally generated by immunization of a transgenic animal (e.g., mouse, rat, hamster, non-human primate, and the like) capable of producing a repertoire of human antibodies, as described herein and/or as known in the art. Cells that produce a human anti-IL-12/23p40 (or anti-IL-23) antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278.

Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, CA), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge antibody Technologies; U.S. Pat. No. 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra, each of the above patents and publications entirely incorporated herein by reference.

Antibodies used in the method of the present invention can also be prepared using at least one anti-IL-12/23p40 (or anti-IL-23) antibody encoding nucleic acid to provide transgenic animals or mammals, such as goats, cows, horses, sheep, rabbits, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Antibodies used in the method of the present invention can additionally be prepared using at least one anti-IL-12/23p40 (or anti-IL-23) antibody encoding nucleic acid to provide transgenic plants and cultured plant cells (e.g., but not limited to, tobacco and maize) that produce such antibodies, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize has been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Antibodies have also been produced in large amounts from transgenic plant seeds including antibody fragments, such as single chain antibodies (scFv's), including tobacco seeds and potato tubers. See, e.g., Conrad et al., Plant Mol. Biol. 38:101-109 (1998) and references cited therein. Thus, antibodies of the present invention can also be produced using transgenic plants, according to known methods. See also, e.g., Fischer et al., Biotechnol. Appl. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. Each of the above references is entirely incorporated herein by reference.

The antibodies used in the method of the invention can bind human IL-12/IL-23p40 or IL-23 with a wide range of affinities (KD). In a preferred embodiment, a human mAb can optionally bind human IL-12/IL-23p40 or IL-23 with high affinity. For example, a human mAb can bind human IL-12/IL-23p40 or IL-23 with a KD equal to or less than about 10-7 M, such as but not limited to, 0.1-9.9 (or any range or value therein)×10-7, 10-8, 10-9, 10-10, 10-11, 10-12, 10-13 or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, NY (1984); Kuby, Janis Immunology, W. H. Freeman and Company: New York, NY (1992); and methods described herein). The measured affinity of a particular antibody-antigen interaction can vary if measured under different conditions (e.g., salt concentration, pH). Thus, measurements of affinity and other antigen-binding parameters (e.g., KD, Ka, Kd) are preferably made with standardized solutions of antibody and antigen, and a standardized buffer, such as the buffer described herein.

Vectors and Host Cells

The present invention also relates to vectors that include isolated nucleic acid molecules, host cells that are genetically engineered with the recombinant vectors, and the production of at least one anti-IL-12/IL-23p40 antibody by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but are not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one antibody used in the method of the present invention can be expressed in a modified form, such as a fusion protein, and can include not only secretion signals, but also additional heterologous functional regions. For instance, a region of additional amino acids, particularly charged amino acids, can be added to the N-terminus of an antibody to improve stability and persistence in the host cell, during purification, or during subsequent handling and storage. Also, peptide moieties can be added to an antibody of the present invention to facilitate purification. Such regions can be removed prior to final preparation of an antibody or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein used in the method of the present invention. Alternatively, nucleic acids can be expressed in a host cell by turning on (by manipulation) in a host cell that contains endogenous DNA encoding an antibody. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the antibodies, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art, and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va (www.atcc.org). Preferred host cells include cells of lymphoid origin, such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to, an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

Purification of an Antibody

An anti-IL-12/IL-23p40 or IL-23 antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, NY, (1997-2001), e.g., Chapters 1, 4, 6, 8, 9, 10, each entirely incorporated herein by reference.

Antibodies used in the method of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

Anti-IL-12/IL-23p40 or IL-23 Antibodies

An anti-IL-12/IL-23p40 or IL-23 antibody according to the present invention includes any protein or peptide containing molecule that comprises at least a portion of an immunoglobulin molecule, such as but not limited to, at least one ligand binding portion (LBP), such as but not limited to, a complementarity determining region (CDR) of a heavy or light chain or a ligand binding portion thereof, a heavy chain or light chain variable region, a framework region (e.g., FR1, FR2, FR3, FR4 or fragment thereof, further optionally comprising at least one substitution, insertion or deletion), a heavy chain or light chain constant region, (e.g., comprising at least one CH1, hinge1, hinge2, hinge3, hinge4, CH2, or CH3 or fragment thereof, further optionally comprising at least one substitution, insertion or deletion), or any portion thereof, that can be incorporated into an antibody. An antibody can include or be derived from any mammal, such as but not limited to, a human, a mouse, a rabbit, a rat, a rodent, a primate, or any combination thereof, and the like.

Preferably, the human antibody or antigen-binding fragment binds human IL-12/IL-23p40 or IL-23 and, thereby, partially or substantially neutralizes at least one biological activity of the protein. An antibody, or specified portion or variant thereof, that partially or preferably substantially neutralizes at least one biological activity of at least one IL-12/IL-23p40 or IL-23 protein or fragment can bind the protein or fragment and thereby inhibit activities mediated through the binding of IL-12/IL-23p40 or IL-23 to the IL-12 and/or IL-23 receptor or through other IL-12/IL-23p40 or IL-23-dependent or mediated mechanisms. As used herein, the term “neutralizing antibody” refers to an antibody that can inhibit an IL-12/IL-23p40 or IL-23-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-IL-12/IL-23p40 or IL-23 antibody to inhibit an IL-12/IL-23p40 or IL-23-dependent activity is preferably assessed by at least one suitable IL-12/IL-23p40 or IL-23 protein or receptor assay, as described herein and/or as known in the art. A human antibody can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4 (e.g., γ1, γ2, γ3, γ4). Antibodies of this type can be prepared by employing a transgenic mouse or other trangenic non-human mammal comprising at least one human light chain (e.g., IgG, IgA, and IgM) transgenes as described herein and/or as known in the art. In another embodiment, the anti-IL-23 human antibody comprises an IgG1 heavy chain and an IgG1 light chain.

An antibody binds at least one specified epitope specific to at least one IL-12/IL-23p40 or IL-23 protein, subunit, fragment, portion or any combination thereof. The at least one epitope can comprise at least one antibody binding region that comprises at least one portion of the protein, which epitope is preferably comprised of at least one extracellular, soluble, hydrophillic, external or cytoplasmic portion of the protein.

Generally, the human antibody or antigen-binding fragment will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. The CDR sequences can be derived from human germline sequences or closely match the germline sequences. For example, the CDRs from a synthetic library derived from the original non-human CDRs can be used. These CDRs can be formed by incorporation of conservative substitutions from the original non-human sequence. In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3.

Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

In one embodiment, an anti-IL-12/23p40 antibody useful for the invention is a monoclonal antibody, preferably a human mAb, comprising heavy chain complementarity determining regions (CDRs) HCDR1, HCDR2, and HCDR3 of SEQ ID NOs: 1, 2, and 3, respectively; and light chain CDRs LCDR1, LCDR2, and LCDR3, of SEQ ID NOs: 4, 5, and 6, respectively.

The anti-IL-12/IL-23p40 or IL-23 specific antibody can comprise at least one of a heavy or light chain variable region having a defined amino acid sequence. For example, in a preferred embodiment, the anti-IL-12/IL-23p40 or IL-23 antibody comprises an anti-IL-12/IL-23p40 antibody with a heavy chain variable region comprising an amino acid sequence at least 85%, preferably at least 90%, more preferably at least 95%, and most preferably 100% identical to SEQ ID NO:7, and a light chain variable region comprising an amino acid sequence at least 85%, preferably at least 90%, more preferably at least 95%, and most preferably 100% identical to SEQ ID NO:8.

The anti-IL-12/IL-23p40 or IL-23 specific antibody can also comprise at least one of a heavy or light chain having a defined amino acid sequence. In another preferred embodiment, the anti-IL-12/IL-23p40 or IL-23 antibody comprises an anti-IL-12/IL-23p40 antibody with a heavy chain comprising an amino acid sequence at least 85%, preferably at least 90%, more preferably at least 95%, and most preferably 100% identical to SEQ ID NO:10, and a light chain variable region comprising an amino acid sequence at least 85%, preferably at least 90%, more preferably at least 95%, and most preferably 100% identical to SEQ ID NO:11.

Preferably, the anti-IL-12/23p40 antibody is ustekinumab (Stelara®), comprising a heavy chain having the amino acid sequence of SEQ ID NO: 10 and a light chain comprising the amino acid sequence of SEQ ID NO: 11. Other examples of anti-IL12/23p40 antibodies useful for the invention include, but are not limited to, Briakinumab (ABT-874, Abbott) and other antibodies described in U.S. Pat. Nos. 6,914,128, 7,247,711, 7,700,739, the entire contents of which are incorporated herein by reference).

The invention also relates to antibodies, antigen-binding fragments, immunoglobulin chains and CDRs comprising amino acids in a sequence that is substantially the same as an amino acid sequence described herein. Preferably, such antibodies or antigen-binding fragments and antibodies comprising such chains or CDRs can bind human IL-12/IL-23p40 or IL-23 with high affinity (e.g., KD less than or equal to about 10-9 M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include, without limitation, replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Antibodies that bind to human IL-12/IL-23p40 or IL-23 and that comprise a defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human IL-12/IL-23p40 or IL-23 or a fragment thereof to elicit the production of antibodies. If desired, the antibody producing cells can be isolated and hybridomas or other immortalized antibody-producing cells can be prepared as described herein and/or as known in the art. Alternatively, the antibody, specified portion or variant can be expressed using the encoding nucleic acid or portion thereof in a suitable host cell.

An anti-IL-12/IL-23p40 or IL-23 antibody used in the method of the present invention can include one or more amino acid substitutions, deletions or additions, either from natural mutations or human manipulation, as specified herein.

The number of amino acid substitutions a skilled artisan would make depends on many factors, including those described above. Generally speaking, the number of amino acid substitutions, insertions or deletions for any given anti-IL-12/IL-23p40 or IL-23 antibody, fragment or variant will not be more than 40, 30, 20, 19, 18, 17, 16, 15, 14, 13, 12, 11, 10, 9, 8, 7, 6, 5, 4, 3, 2, 1, such as 1-30 or any range or value therein, as specified herein.

Amino acids in an anti-IL-12/IL-23p40 or IL-23 specific antibody that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to, at least one IL-12/IL-23p40 or IL-23 neutralizing activity. Sites that are critical for antibody binding can also be identified by structural analysis, such as crystallization, nuclear magnetic resonance or photoaffinity labeling (Smith, et al., J. Mol. Biol. 224:899-904 (1992) and de Vos, et al., Science 255:306-312 (1992)).

Anti-IL-12/IL-23p40 or IL-23 antibodies can include, but are not limited to, at least one portion, sequence or combination selected from 5 to all of the contiguous amino acids of at least one of SEQ ID NOs 1, 2, 3, 4, 5, 6, 7, 8, 10, or 11.

IL-12/IL-23p40 or IL-23 antibodies or specified portions or variants can include, but are not limited to, at least one portion, sequence or combination selected from at least 3-5 contiguous amino acids of the SEQ ID NOs above; 5-17 contiguous amino acids of the SEQ ID NOs above, 5-10 contiguous amino acids of the SEQ ID NOs above, 5-11 contiguous amino acids of the SEQ ID NOs above, 5-7 contiguous amino acids of the SEQ ID NOs above; 5-9 contiguous amino acids of the SEQ ID NOs above.

An anti-IL-12/IL-23p40 or IL-23 antibody can further optionally comprise a polypeptide of at least one of 70-100% of 5, 17, 10, 11, 7, 9, 119, 108, 449, or 214 contiguous amino acids of the SEQ ID NOs above. In one embodiment, the amino acid sequence of an immunoglobulin chain, or portion thereof (e.g., variable region, CDR) has about 70-100% identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or any range or value therein) to the amino acid sequence of the corresponding chain of at least one of the SEQ ID NOs above. For example, the amino acid sequence of a light chain variable region can be compared with the sequence of the SEQ ID NOs above, or the amino acid sequence of a heavy chain CDR3 can be compared with the SEQ ID NOs above. Preferably, 70-100% amino acid identity (i.e., 90, 91, 92, 93, 94, 95, 96, 97, 98, 99, 100 or any range or value therein) is determined using a suitable computer algorithm, as known in the art.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, MD).

Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm can also be used to determine identity.

Exemplary heavy chain and light chain variable regions sequences and portions thereof are provided in the SEQ ID NOs above. The antibodies of the present invention, or specified variants thereof, can comprise any number of contiguous amino acid residues from an antibody of the present invention, wherein that number is selected from the group of integers consisting of from 10-100% of the number of contiguous residues in an anti-IL-12/IL-23p40 or IL-23 antibody. Optionally, this subsequence of contiguous amino acids is at least about 10, 20, 30, 40, 50, 60, 70, 80, 90, 100, 110, 120, 130, 140, 150, 160, 170, 180, 190, 200, 210, 220, 230, 240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active antibody of the present invention. Biologically active antibodies have a specific activity at least 20%, 30%, or 40%, and, preferably, at least 50%, 60%, or 70%, and, most preferably, at least 80%, 90%, or 95%-100% or more (including, without limitation, up to 10 times the specific activity) of that of the native (non-synthetic), endogenous or related and known antibody. Methods of assaying and quantifying measures of enzymatic activity and substrate specificity are well known to those of skill in the art.

In another aspect, the invention relates to human antibodies and antigen-binding fragments, as described herein, which are modified by the covalent attachment of an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified antibodies and antigen-binding fragments can comprise one or more organic moieties that are covalently bonded, directly or indirectly, to the antibody. Each organic moiety that is bonded to an antibody or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the invention. Hydrophilic polymers suitable for modifying antibodies of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody of the invention has a molecular weight of about 800 to about 150,000 Daltons as a separate molecular entity. For example, PEG5000 and PEG20,000, wherein the subscript is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N, N-carbonyl diimidazole) on a fatty acid or fatty acid ester can be coupled to a hydroxyl group on a polymer.

Fatty acids and fatty acid esters suitable for modifying antibodies of the invention can be saturated or can contain one or more units of unsaturation. Fatty acids that are suitable for modifying antibodies of the invention include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-Δ9-octadecanoate (C18, oleate), all cis-A5,8,11,14-eicosatetraenoate (C20, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably, one to about six, carbon atoms.

The modified human antibodies and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups, such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, CA (1996)). An activating group can be bonded directly to the organic group (e.g., hydrophilic polymer, fatty acid, fatty acid ester), or through a linker moiety, for example, a divalent C1-C12 group wherein one or more carbon atoms can be replaced by a heteroatom, such as oxygen, nitrogen or sulfur. Suitable linker moieties include, for example, tetraethylene glycol, —(CH2)3-, —NH—(CH2)6-NH—, —(CH2)2-NH— and —CH2-O—CH2-CH2-O—CH2-CH2O-CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate, as described, or can be reacted with maleic anhydride and the resulting product cyclized to produce an activated maleimido derivative of the fatty acid. (See, for example, Thompson, et al., WO 92/16221, the entire teachings of which are incorporated herein by reference.)

The modified antibodies can be produced by reacting a human antibody or antigen-binding fragment with a modifying agent. For example, the organic moieties can be bonded to the antibody in a non-site specific manner by employing an amine-reactive modifying agent, for example, an NHS ester of PEG. Modified human antibodies or antigen-binding fragments can also be prepared by reducing disulfide bonds (e.g., intra-chain disulfide bonds) of an antibody or antigen-binding fragment. The reduced antibody or antigen-binding fragment can then be reacted with a thiol-reactive modifying agent to produce the modified antibody of the invention. Modified human antibodies and antigen-binding fragments comprising an organic moiety that is bonded to specific sites of an antibody of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, CA (1996).

The method of the present invention also uses an anti-IL-12/IL-23p40 or IL-23 antibody composition comprising at least one, at least two, at least three, at least four, at least five, at least six or more anti-IL-12/IL-23p40 or IL-23 antibodies thereof, as described herein and/or as known in the art that are provided in a non-naturally occurring composition, mixture or form. Such compositions comprise non-naturally occurring compositions comprising at least one or two full length, C- and/or N-terminally deleted variants, domains, fragments, or specified variants, of the anti-IL-12/IL-23p40 or IL-23 antibody amino acid sequence selected from the group consisting of 70-100% of the contiguous amino acids of the SEQ ID NOs above, or specified fragments, domains or variants thereof. Preferred anti-IL-12/IL-23p40 or IL-23 antibody compositions include at least one or two full length, fragments, domains or variants as at least one CDR or LBP containing portions of the anti-IL-12/IL-23p40 or IL-23 antibody sequence described herein, for example, 70-100% of the SEQ ID NOs above, or specified fragments, domains or variants thereof. Further preferred compositions comprise, for example, 40-99% of at least one of 70-100% of the SEQ ID NOs above, etc., or specified fragments, domains or variants thereof. Such composition percentages are by weight, volume, concentration, molarity, or molality as liquid or dry solutions, mixtures, suspension, emulsions, particles, powder, or colloids, as known in the art or as described herein.

Antibody Compositions Comprising Further Therapeutically Active Ingredients

The antibody compositions used in the method of the invention can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21st edition, Springhouse Corp., Springhouse, P A, 2001; Health Professional's Drug Guide 2001, ed., Shannon, Wilson, Stang, Prentice-Hall, Inc, Upper Saddle River, NJ; Pharmcotherapy Handbook, Wells et al., ed., Appleton & Lange, Stamford, CT, each entirely incorporated herein by reference).

By way of example of the drugs that can be combined with the antibodies for the method of the present invention, the anti-infective drug can be at least one selected from amebicides or at least one antiprotozoals, anthelmintics, antifungals, antimalarials, antituberculotics or at least one antileprotics, aminoglycosides, penicillins, cephalosporins, tetracyclines, sulfonamides, fluoroquinolones, antivirals, macrolide anti-infectives, and miscellaneous anti-infectives. The hormonal drug can be at least one selected from corticosteroids, androgens or at least one anabolic steroid, estrogen or at least one progestin, gonadotropin, antidiabetic drug or at least one glucagon, thyroid hormone, thyroid hormone antagonist, pituitary hormone, and parathyroid-like drug. The at least one cephalosporin can be at least one selected from cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, cefuroxime axetil, cefuroxime sodium, cephalexin hydrochloride, cephalexin monohydrate, cephradine, and loracarbef.

The at least one coricosteroid can be at least one selected from betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, and triamcinolone diacetate. The at least one androgen or anabolic steroid can be at least one selected from danazol, fluoxymesterone, methyltestosterone, nandrolone decanoate, nandrolone phenpropionate, testosterone, testosterone cypionate, testosterone enanthate, testosterone propionate, and testosterone transdermal system.

The at least one immunosuppressant can be at least one selected from azathioprine, basiliximab, cyclosporine, daclizumab, lymphocyte immune globulin, muromonab-CD3, mycophenolate mofetil, mycophenolate mofetil hydrochloride, sirolimus, 6-mercaptopurine, methotrexate, mizoribine, and tacrolimus.

The at least one local anti-infective can be at least one selected from acyclovir, amphotericin B, azelaic acid cream, bacitracin, butoconazole nitrate, clindamycin phosphate, clotrimazole, econazole nitrate, erythromycin, gentamicin sulfate, ketoconazole, mafenide acetate, metronidazole (topical), miconazole nitrate, mupirocin, naftifine hydrochloride, neomycin sulfate, nitrofurazone, nystatin, silver sulfadiazine, terbinafine hydrochloride, terconazole, tetracycline hydrochloride, tioconazole, and tolnaftate. The at least one scabicide or pediculicide can be at least one selected from crotamiton, lindane, permethrin, and pyrethrins. The at least one topical corticosteroid can be at least one selected from betamethasone dipropionate, betamethasone valerate, clobetasol propionate, desonide, desoximetasone, dexamethasone, dexamethasone sodium phosphate, diflorasone diacetate, fluocinolone acetonide, fluocinonide, flurandrenolide, fluticasone propionate, halcionide, hydrocortisone, hydrocortisone acetate, hydrocortisone butyrate, hydrocorisone valerate, mometasone furoate, and triamcinolone acetonide. (See, e.g., pp. 1098-1136 of Nursing 2001 Drug Handbook.)

Anti-IL-12/IL-23p40 or IL-23 antibody compositions can further comprise at least one of any suitable and effective amount of a composition or pharmaceutical composition comprising at least one anti-IL-12/IL-23p40 or IL-23 antibody contacted or administered to a cell, tissue, organ, animal or subject in need of such modulation, treatment or therapy, optionally further comprising at least one selected from at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept, CDP-571, CDP-870, afelimomab, lenercept, and the like), an antirheumatic (e.g., methotrexate, auranofin, aurothioglucose, azathioprine, etanercept, gold sodium thiomalate, hydroxychloroquine sulfate, leflunomide, sulfasalzine), an immunization, an immunoglobulin, an immunosuppressive (e.g., azathioprine, basiliximab, cyclosporine, daclizumab), a cytokine or a cytokine antagonist. Non-limiting examples of such cytokines include, but are not limited to, any of IL-1 to IL-23 et al. (e.g., IL-1, IL-2, etc.). Suitable dosages are well known in the art. See, e.g., Wells et al., eds., Pharmacotherapy Handbook, 2nd Edition, Appleton and Lange, Stamford, C T (2000); PDR Pharmacopoeia, Tarascon Pocket Pharmacopoeia 2000, Deluxe Edition, Tarascon Publishing, Loma Linda, CA (2000), each of which references are entirely incorporated herein by reference.

Anti-IL-12/IL-23p40 or IL-23 antibody compounds, compositions or combinations used in the method of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18th Edition, Mack Publishing Co. (Easton, PA) 1990. Pharmaceutically acceptable carriers can be routinely selected that are suitable for the mode of administration, solubility and/or stability of the anti-IL-12/IL-23p40, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Anti-IL-12/IL-23p40 or IL-23 antibody compositions can also include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, anti-IL-12/IL-23p40 or IL-23 antibody compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the anti-IL-12/IL-23p40 or IL-23 antibody, portion or variant compositions according to the invention are known in the art, e.g., as listed in “Remington: The Science & Practice of Pharmacy,” 19th ed., Williams & Williams, (1995), and in the “Physician's Desk Reference,” 52nd ed., Medical Economics, Montvale, NJ (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which can be useful for intraarticular delivery.

Formulations

As noted above, the invention provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one anti-IL-12/IL-23p40 or IL-23 antibody in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the method of the invention uses an article of manufacture, comprising packaging material and at least one vial comprising a solution of at least one anti-IL-12/IL-23p40 or IL-23 antibody with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further uses an article of manufacture, comprising packaging material, a first vial comprising lyophilized anti-IL-12/IL-23p40 or IL-23 antibody, and a second vial comprising an aqueous diluent of prescribed buffer or preservative, wherein said packaging material comprises a label that instructs a subject to reconstitute the anti-IL-12/IL-23p40 or IL-23 antibody in the aqueous diluent to form a solution that can be held over a period of twenty-four hours or greater.

The anti-IL-12/IL-23p40 or IL-23 antibody used in accordance with the present invention can be produced by recombinant means, including from mammalian cell or transgenic preparations, or can be purified from other biological sources, as described herein or as known in the art.

The range of the anti-IL-12/IL-23p40 or IL-23 antibody includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations can be prepared by a process which comprises mixing at least one anti-IL-12/IL-23p40 or IL-23 antibody and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one anti-IL-12/IL-23p40 or IL-23 specific antibody and preservative in an aqueous diluent is carried out using conventional dissolution and mixing procedures. To prepare a suitable formulation, for example, a measured amount of at least one anti-IL-12/IL-23p40 or IL-23 antibody in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The formulations can be provided to subjects as clear solutions or as dual vials comprising a vial of lyophilized anti-IL-12/IL-23p40 or IL-23 specific antibody that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of subject treatment and thus can provide a more convenient treatment regimen than currently available.

The present articles of manufacture are useful for administration over a period ranging from immediate to twenty-four hours or greater. Accordingly, the presently claimed articles of manufacture offer significant advantages to the subject. Formulations of the invention can optionally be safely stored at temperatures of from about 2° C. to about 40° C. and retain the biologically activity of the protein for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of anti-IL-12/IL-23p40 or IL-23 specific antibody can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to subjects as clear solutions or as dual vials comprising a vial of lyophilized at least one anti-IL-12/IL-23p40 or IL-23 specific antibody that is reconstituted with a second vial containing the aqueous diluent. Either a single solution vial or dual vial requiring reconstitution can be reused multiple times and can suffice for a single or multiple cycles of subject treatment and thus provides a more convenient treatment regimen than currently available.

The claimed products can be provided indirectly to subjects by providing to pharmacies, clinics, or other such institutions and facilities, clear solutions or dual vials comprising a vial of lyophilized at least one anti-IL-12/IL-23p40 or IL-23 specific antibody that is reconstituted with a second vial containing the aqueous diluent. The clear solution in this case can be up to one liter or even larger in size, providing a large reservoir from which smaller portions of the at least one antibody solution can be retrieved one or multiple times for transfer into smaller vials and provided by the pharmacy or clinic to their customers and/or subjects.

Recognized devices comprising single vial systems include pen-injector devices for delivery of a solution, such as BD Pens, BD Autojector®, Humaject®, NovoPen®, B-D® Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, Smartject® e.g., as made or developed by Becton Dickensen (Franklin Lakes, NJ, www.bectondickenson.com), Disetronic (Burgdorf, Switzerland, www.disetronic.com; Bioject, Portland, Oregon (www.bioject.com); National Medical Products, Weston Medical (Peterborough, UK, www.weston-medical.com), Medi-Ject Corp (Minneapolis, MN, www.mediject.com), and similarly suitable devices. Recognized devices comprising a dual vial system include those pen-injector systems for reconstituting a lyophilized drug in a cartridge for delivery of the reconstituted solution, such as the HumatroPen®. Examples of other devices suitable include pre-filled syringes, auto-injectors, needle free injectors, and needle free IV infusion sets.

The products can include packaging material. The packaging material provides, in addition to the information required by the regulatory agencies, the conditions under which the product can be used. The packaging material of the present invention provides instructions to the subject, as applicable, to reconstitute the at least one anti-IL-12/IL-23p40 or IL-23 antibody in the aqueous diluent to form a solution and to use the solution over a period of 2-24 hours or greater for the two vial, wet/dry, product. For the single vial, solution product, pre-filled syringe or auto-injector, the label indicates that such solution can be used over a period of 2-24 hours or greater. The products are useful for human pharmaceutical product use.

The formulations used in the method of the present invention can be prepared by a process that comprises mixing an anti-IL-12/IL-23p40 and a selected buffer, preferably, a phosphate buffer containing saline or a chosen salt. Mixing the anti-IL-12/IL-23p40 antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The method of the invention provides pharmaceutical compositions comprising various formulations useful and acceptable for administration to a human or animal subject. Such pharmaceutical compositions are prepared using water at “standard state” as the diluent and routine methods well known to those of ordinary skill in the art. For example, buffering components such as histidine and histidine monohydrochloride hydrate, can be provided first followed by the addition of an appropriate, non-final volume of water diluent, sucrose and polysorbate 80 at “standard state.” Isolated antibody can then be added. Last, the volume of the pharmaceutical composition is adjusted to the desired final volume under “standard state” conditions using water as the diluent. Those skilled in the art will recognize a number of other methods suitable for the preparation of the pharmaceutical compositions.

The pharmaceutical compositions can be aqueous solutions or suspensions comprising the indicated mass of each constituent per unit of water volume or having an indicated pH at “standard state.” As used herein, the term “standard state” means a temperature of 25° C.+/−2° C. and a pressure of 1 atmosphere. The term “standard state” is not used in the art to refer to a single art recognized set of temperatures or pressure, but is instead a reference state that specifies temperatures and pressure to be used to describe a solution or suspension with a particular composition under the reference “standard state” conditions. This is because the volume of a solution is, in part, a function of temperature and pressure. Those skilled in the art will recognize that pharmaceutical compositions equivalent to those disclosed here can be produced at other temperatures and pressures. Whether such pharmaceutical compositions are equivalent to those disclosed here should be determined under the “standard state” conditions defined above (e.g. 25° C.+/−2° C. and a pressure of 1 atmosphere).

Importantly, such pharmaceutical compositions can contain component masses “about” a certain value (e.g. “about 0.53 mg L-histidine”) per unit volume of the pharmaceutical composition or have pH values about a certain value. A component mass present in a pharmaceutical composition or pH value is “about” a given numerical value if the isolated antibody present in the pharmaceutical composition is able to bind a peptide chain while the isolated antibody is present in the pharmaceutical composition or after the isolated antibody has been removed from the pharmaceutical composition (e.g., by dilution). Stated differently, a value, such as a component mass value or pH value, is “about” a given numerical value when the binding activity of the isolated antibody is maintained and detectable after placing the isolated antibody in the pharmaceutical composition.

Competition binding analysis is performed to determine if the IL-12/IL-23p40 or IL-23 specific mAbs bind to similar or different epitopes and/or compete with each other. Abs are individually coated on ELISA plates. Competing mAbs are added, followed by the addition of biotinylated hrIL-12 or IL-23. For positive control, the same mAb for coating can be used as the competing mAb (“self-competition”). IL-12/IL-23p40 or IL-23 binding is detected using streptavidin. These results demonstrate whether the mAbs recognize similar or partially overlapping epitopes on IL-12/IL-23p40 or IL-23.

In one embodiment of the pharmaceutical compositions, the isolated antibody concentration is from about 77 to about 104 mg per ml of the pharmaceutical composition. In another embodiment of the pharmaceutical compositions the pH is from about 5.5 to about 6.5.

The stable or preserved formulations can be provided to subjects as clear solutions or as dual vials comprising a vial of lyophilized at least one anti-IL-12/IL-23p40 that is reconstituted with a second vial containing a preservative or buffer and excipients in an aqueous diluent. Either a single solution vial or dual vial requiring reconstitution can be reused multiple times and can suffice for a single or multiple cycles of subject treatment and thus provides a more convenient treatment regimen than currently available.

Other formulations or methods of stabilizing the anti-IL-12/IL-23p40 can result in other than a clear solution of lyophilized powder comprising the antibody. Among non-clear solutions are formulations comprising particulate suspensions, said particulates being a composition containing the anti-IL-12/IL-23p40 in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of glelatin agar, starch, arabinogalactan, albumin, collagen, polyglycolic acid, polylactic aced, glycolide-L(−) lactide poly(episilon-caprolactone, poly(epsilon-caprolactone-CO-lactic acid), poly(epsilon-caprolactone-CO-glycolic acid), poly(ß-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(−) lactide poly(episilon-caprolactone, poly(epsilon-caprolactone-CO-lactic acid), and poly(epsilon-caprolactone-CO-glycolic acid. Solvents useful for dissolving the polymer and/or the active include: water, hexafluoroisopropanol, methylenechloride, tetrahydrofuran, hexane, benzene, or hexafluoroacetone sesquihydrate. The process of dispersing the active containing phase with a second phase can include pressure forcing said first phase through an orifice in a nozzle to affect droplet formation.

Dry powder formulations can result from processes other than lyophilization, such as by spray drying or solvent extraction by evaporation or by precipitation of a crystalline composition followed by one or more steps to remove aqueous or non-aqueous solvent. Preparation of a spray-dried antibody preparation is taught in U.S. Pat. No. 6,019,968. The antibody-based dry powder compositions can be produced by spray drying solutions or slurries of the antibody and, optionally, excipients, in a solvent under conditions to provide a respirable dry powder. Solvents can include polar compounds, such as water and ethanol, which can be readily dried. Antibody stability can be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions can be administered to the lung of a subject using a metered dose inhaler. Equipment useful in the commercial manufacture of spray dried medicaments are manufactured by Buchi Ltd. or Niro Corp.

An anti-IL-12/IL-23p40 in either the stable or preserved formulations or solutions described herein, can be administered to a subject in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

Therapeutic Applications

The present invention also provides a method for modulating or treating ulcerative colitis, in a cell, tissue, organ, animal, or subject, as known in the art or as described herein, using at least one IL-23 antibody of the present invention, e.g., administering or contacting the cell, tissue, organ, animal, or subject with a therapeutic effective amount of IL-12/IL-23p40 or IL-23 specific antibody.

Any method of the present invention can comprise administering an effective amount of a composition or pharmaceutical composition comprising an IL-12/IL-23p40 to a cell, tissue, organ, animal or subject in need of such modulation, treatment or therapy. Such a method can optionally further comprise co-administration or combination therapy for treating such diseases or disorders, wherein the administering of said at least one IL-12/IL-23p40, specified portion or variant thereof, further comprises administering, before concurrently, and/or after, at least one selected from at least one TNF antagonist (e.g., but not limited to, a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept (Enbrel™), adalimulab (Humira™), CDP-571, CDP-870, afelimomab, lenercept, and the like), an antirheumatic (e.g., methotrexate, auranofin, aurothioglucose, azathioprine, gold sodium thiomalate, hydroxychloroquine sulfate, leflunomide, sulfasalzine), a muscle relaxant, a narcotic, a non-steroid anti-inflammatory drug (NSAID) (e.g., 5-aminosalicylate), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2nd Edition, Appleton and Lange, Stamford, C T (2000); PDR Pharmacopoeia, Tarascon Pocket Pharmacopoeia 2000, Deluxe Edition, Tarascon Publishing, Loma Linda, C A (2000); Nursing 2001 Handbook of Drugs, 21st edition, Springhouse Corp., Springhouse, P A, 2001; Health Professional's Drug Guide 2001, ed., Shannon, Wilson, Stang, Prentice-Hall, Inc, Upper Saddle River, NJ, each of which references are entirely incorporated herein by reference.

Therapeutic Treatments

Treatment of ulcerative colitis is affected by administering an effective amount or dosage of an anti-IL-12/23p40 composition in a subject in need thereof. The dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

In one exemplary regimen of providing safe and effective treatment of severely active UC in a subject in need thereof, a total dosage of about 130 mg of an anti-IL-12/IL-23p40 antibody is administered intravenously to the subject per administration. For example, the total volume of the composition administered is appropriately adjusted to provide to the subject the target dosage of the antibody at 80 mg, 90 mg, 100 mg, 110 mg, 120 mg, 130 mg, 140 mg, 150 mg, 160 mg, 170 mg or 180 mg per administration.

In another exemplary regimen of providing safe and effective treatment of severely active UC in a subject in need thereof, a total dosage of about 6.0 mg/kg±1.5 mg/kg of an anti-IL-12/IL-23p40 antibody is administered intravenously to the subject per administration. For example, the total volume of the composition administered is appropriately adjusted to provide to the subject the target dosage of the antibody at 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, or 9.0 mg/kg body weight of the subject per administration.

The total dosage of an anti-IL-12/IL-23p40 antibody to be administered to the subject per administration can be administered by intravenous infusion over a period of about 30 minutes to 180 minutes, preferably 60 minutes to 120 minutes, such as 30 minutes, 60 minutes, 90 minutes, 120 minutes, 150 minutes, or 180 minutes.

In yet another exemplary regimen of providing safe and effective treatment of severely active UC in a subject in need thereof, a total dosage of about 90 mg of an anti-IL-12/IL-23p40 antibody is administered subcutaneously to the subject per administration. For example, the total volume of the composition administered is appropriately adjusted to provide to the subject the target dosage of the antibody at 40 mg, 50 mg, 60 mg, 70 mg, 80 mg, 90 mg, 100 mg, 110 mg, 120 mg, 130 mg or 140 mg per administration. The target dosage per administration can be administered in a single subcutaneous injection or in multiple subcutaneous injections, such as 1, 2, 3, 4, 5, or more subcutaneous injections.

The total dosage of the anti-IL-12/IL-23p40 antibody can be administered once per day, once per week, once per month, once every six months, etc. for a period of one day, one week, one month, six months, 1 year, 2 years or longer. Multiple administrations of the anti-IL-12/IL-23p40 antibody, each at a total dosage of described herein, can be administered to a subject in need thereof.

Dosage forms (composition) suitable for internal administration generally contain from about 0.001 milligram to about 500 milligrams of active ingredient per unit or container.

For parenteral administration, the antibody can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Many known and developed modes can be used according to the present invention for administering pharmaceutically effective amounts of an IL-12/IL-23p40 antibody. IL-12/IL-23p40 or IL-23 antibodies of the present invention can be delivered in a carrier, as a solution, emulsion, colloid, or suspension, or as a dry powder, using any of a variety of devices and methods suitable for administration by inhalation or other modes described here within or known in the art.

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

Alternative Delivery

The invention further relates to the administration of an anti-IL-12/IL-23p40 or IL-23 antibody by parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. An anti-IL-12/IL-23p40 or IL-23 antibody composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

### Embodiments

The invention provides also the following non-limiting embodiments.
1. A method of treating moderately to severely active ulcerative colitis (UC) in a subject in need thereof, comprising administering to the subject a pharmaceutical composition comprising a clinically proven safe and clinically proven effective amount of an anti-IL-12/IL-23p40 antibody, wherein the antibody comprises a heavy chain variable region and a light chain variable region, the heavy chain variable region comprising: a complementarity determining region heavy chain 1 (CDRH1) amino acid sequence of SEQ ID NO:1; a CDRH2 amino acid sequence of SEQ ID NO:2; and a CDRH3 amino acid sequence of SEQ ID NO:3; and the light chain variable region comprising: a complementarity determining region light chain 1 (CDRL1) amino acid sequence of SEQ ID NO:4; a CDRL2 amino acid sequence of SEQ ID NO:5; and a CDRL3 amino acid sequence of SEQ ID NO:6, wherein the antibody is administered intravenously to the subject, preferably at week 0 of the treatment, at a dosage of about 6.0 mg/kg body weight of the subject or 130 mg per administration, administered subcutaneously to the subject, preferably at week 8 of the treatment, at a dosage of about 90 mg per administration, and the antibody is administered in a maintenance dose every 8 weeks after the treatment at week 8 or every 12 weeks after the treatment at week 8, wherein the subject is a responder to treatment at week 92 based on satisfying one or more clinical endpoints selected from the group consisting of:
   (a) symptomatic remission;
   (b) partial Mayo remission;
   (c) Mayo rectal bleeding subscore of 0;
   (d) Mayo stool frequency subscore of 0 or 1;
   (e) mean absolute stool numbers decreased by at least 3;
   (f) decrease in corticosteroid usage and/or dosage;
   (g) corticosteroid-free symptomatic remission;
   (h) corticosteroid-free partial mayo remission;
   (i) normalized fecal lactoferrin;
   (j) normalization of fecal calprotectin levels;
   (k) ≥16-point improvement from induction baseline in the total Inflammatory Bowel Disease Questionnaire (IBDQ) score;
   (l) IBDQ remission;
   (m) a ≥5-point improvement from induction baseline in the SF-36 PCS score; and
   (n) a ≥5-point improvement from induction baseline in the SF-36 MCS score.
2. The method of embodiment 1, wherein the antibody comprises the heavy chain variable region of the amino acid sequence of SEQ ID NO:7 and the light chain variable region of the amino acid sequence of SEQ ID NO:8.
3. The method of embodiment 1, wherein the antibody comprises a heavy chain of the amino acid sequence of SEQ ID NO:10 and a light chain of the amino acid sequence of SEQ ID NO:11.
4. The method of any one of embodiments 1 to 3, wherein the subject had previously failed or were intolerant of at least one therapy selected from the group consisting of an anti-TNF, vedolizumab, corticosteroids, azathioprine (AZA), and 6 mercaptopurine (6 MP), or the subject had demonstrated corticosteroid dependence.
5. The method of any one of embodiments 1 to 3, wherein the subject is in corticosteroid-free clinical remission at least 92 weeks after week 0.
6. The method of any one of embodiments 1 to 3, wherein the subject is a responder to the treatment with the antibody and is identified as having an endoscopic healing continuing at least 92 weeks after week 0.
7. The method of any one of embodiments 1-6, wherein the pharmaceutical composition for intravenous administration further comprises a solution comprising 10 mM L-histidine, 8.5% (w/v) sucrose, 0.04% (w/v) polysorbate 80, 0.4 mg/mL L-methionine, and 20 μg/mL EDTA disodium salt, dehydrate, at pH 6.0.
8. The method of any one of embodiments 1-7, wherein the pharmaceutical composition for subcutaneous administration further comprises a solution comprising 6.7 mM L-histidine, 7.6% (w/v) sucrose, 0.004% (w/v) polysorbate 80, at pH 6.0.
   Having generally described the invention, the same will be more readily understood by reference to the following Examples, which are provided by way of illustration and are not intended as limiting. Further details of the invention are illustrated by the following non-limiting Examples. The disclosures of all citations in the specification are expressly incorporated herein by reference.

### EXAMPLES

### Example 1: Induction Study of Ustekinumab in the Treatment of Ulcerative Colitis in Humans

The following multicenter, randomized, double-blind, placebo-controlled, clinical study in adult men and women with moderately to severely active ulcerative colitis (UC) was performed: A Phase 3, Randomized, Double-blind, Placebo-controlled, Parallel-group, Multicenter Study to Evaluate the Safety and Efficacy of ustekinumab Induction and Maintenance Therapy in Subjects with Moderately to Severely Active Ulcerative Colitis

Overall Rationale

A study was performed to assess the efficacy of intravenous (IV) administration of ustekinumab in subjects with moderately to severely active ulcerative colitis who demonstrated inadequate response or failure to tolerate conventional (corticosteroids or 6-mercaptopurine/azathioprine [6-MP/AZA]) or biologic therapy (TNF antagonist and/or the integrin antagonist, vedolizumab). Subjects received a single 130 mg, a single 6 mg/kg IV dose, or placebo at Week 0. Subjects who demonstrated no clinical response at Week 8 received an additional IV or subcutaneous (SC) dose at Week 8.

Objectives

The primary objectives of the study included (1) evaluating the efficacy of ustekinumab in inducing clinical remission in subjects with moderately to severely active UC; and (2) evaluating the safety of the IV ustekinumab in subjects with moderately to severely active UC.

The secondary objectives of the study included (1) evaluating the efficacy of IV ustekinumab in inducing endoscopic healing (i.e. improvement in the endoscopic appearance of mucosa) in subjects with moderately to severely active UC; (2) evaluating the efficacy of IV ustekinumab in inducing clinical response in subjects with moderately to severely active UC; (3) evaluating the impact of IV ustekinumab on disease-specific health-related quality of life; (4) evaluating the efficacy of ustekinumab treatment on mucosal healing (i.e, endoscopic healing and histologic healing); (5) evaluating the efficacy of induction therapy with IV ustekinumab by biologic failure status; and (6) evaluating the pharmacokinetics (PK), immunogenicity, and pharmacodynamics (PD) of ustekinumab induction therapy in subjects with moderately to severely active UC, including changes in C-reactive protein (CRP), fecal calprotectin, fecal lactoferrin, and other PD biomarkers.

The exploratory objectives of the study included (1) evaluating response using the Mayo score without the physician's global assessment (PGA) subscore and (2) evaluating the performance of the Bristol Stool Form Scale (BSFS) score.

Experimental Design

The Phase 3 development program for ustekinumab comprised 2 separate studies, an induction study and a maintenance study. In the induction study, subjects were randomized at Week 0 into one of three treatment groups: placebo, low-dose ustekinumab, and high-dose ustekinumab. At Week 8, all subjects were evaluated for the primary endpoint of clinical remission and clinical response. Subjects who achieved a clinical response at Week 8 were eligible to enter the maintenance study. Subjects who did not achieve clinical response at Week 8 received a second dose of ustekinumab at Week 8 of treatment.

At Week 16, subjects who did not achieve clinical response at Week 8 were re-evaluated for clinical response. Subjects who achieved clinical response at Week 16 were eligible to enter the maintenance study. Subjects who did not achieve clinical response at Week 16 were not eligible to enter the maintenance study and had a safety follow-up visit approximately 20 weeks after their last dose of study agent (Week 8).

Subjects who were in clinical response to IV ustekinumab during induction comprised the primary population in the maintenance study. The maintenance study is a randomized withdrawal study designed to evaluate maintenance therapy using SC ustekinumab and is currently ongoing.

Dosage and Administration

Subjects received a single IV dose of ustekinumab or placebo at Week 0 of the study. The induction study antibodies with the administered doses are as follows:
Ustekinumab at a low, fixed does of 130 mg
Ustekinumab at a high, weight-range based dose of ˜6 mg/kg:
   Ustekinumab 260 mg (body-weight ≤55 kg)
   Ustekinumab 390 mg (body-weight >55 kg but ≤85 kg)
   Ustekinumab 520 mg (body-weight >85 kg)

Subjects who did not present a clinical response received a second dose of ustekinumab at Week 8. The study antibodies with the second administered doses are as follows:
Subjects who were randomized to placebo at Week 0 received 1 dose of ustekinumab ˜6 mg/kg IV+placebo SC (to maintain the blind) at Week 8.
Subjects who were randomized to ustekinumab at Week 0 received 1 dose of ustekinumab 90 mg SC+placebo IV (to maintain the blind) at Week 8.

Safety Evaluations

Safety was evaluated based on AEs and clinical laboratory test results (i.e., hematology and serum chemistry). Adverse events were either voluntarily reported by the subject or were obtained by means of interviewing subjects in a non-directed manner at study visits. Safety evaluations included the following clinical laboratory tests:
Hematology: Hemoglobin (Hb), hematocrit, red blood cell count, white blood cell (WBC) count, and platelets.
Serum Chemistry: Sodium, potassium, chloride, blood urea nitrogen (BUN), creatinine, aspartate aminotransferase (AST), alanine aminotransferase (ALT), total and direct bilirubin, alkaline phosphatase, calcium, phosphate, albumin, total protein.
Screening: Serology for human immunodeficiency virus antibody, serology for hepatitis C virus (HCV) antibody, serology for hepatitis B virus (HBV) antibody, hepatitis B surface antigen, HBV surface antibody (anti-HBs), and HBV core (anti-HBc) antibody total, QuantiFERON-TB Gold test, pregnancy (β human chorionic gonadotropin [β-HCG]).

Pharmacokinetics

Blood samples for the measurement of serum ustekinumab concentrations were collected at Week 0 (pre- and postinfusion) and Weeks 2, 4, and 8. Analyses of serum ustekinumab concentrations were performed using a validated electrochemiluminescent immunoassay (ECLIA) method on the Meso Scale Discovery (MSD®) platform (Gaithersburg, MD, USA). The lowest quantifiable concentration in a sample for the ECLIA method using the MSD

platform was 0.1688 μg/mL.

Immunogenicity

Antibodies to ustekinumab were evaluated using serum samples collected from all subjects. Analyses of antibodies to ustekinumab were performed using a validated, drug-tolerant, electrochemiluminescence immunoassay (ECLIA), in which ustekinumab was used to capture and detect induced immune responses to ustekinumab. Antibody titers were determined for all subjects who had antibodies to ustekinumab and the neutralizing antibody (Nab) status of anti-drug antibody positive samples were determined.

Efficacy Evaluation

Efficacy evaluations were collected throughout the study. Mayo score and partial Mayo score, Ulcerative Colitis Endoscopic Index of Severity (UCEIS), Bristol Stool Form Scale (BSFS), C-reactive protein (CRP), fecal lactoferrin, fecal calprotectin, Inflammatory Bowel Disease Questionnaire (IBDQ), 36-item Short Form Health Survey (SF-36), and EuroQoL-5D Health Questionnaire were all evaluated to determine efficacy. The efficacy criteria were defined as follows:
Clinical remission (global submissions): Mayo score ≤2 points, with no individual subscore >1.
Clinical remission (US submissions): absolute stool number ≤3, rectal bleeding subscore of 0, and Mayo endoscopy subscore of 0 or 1.
Clinical response: a decrease from induction baseline in the Mayo score by ≥30% and ≥3 points, with either a decrease from baseline in the rectal bleeding subscore ≥1 or a rectal bleeding subscore of 0 or 1.
Endoscopic healing (i.e., improvement in the endoscopic appearance of the mucosa): Mayo endoscopy subscore of 0 or 1.
Histologic healing: based on the Geboes score and is defined as 0 to <5% neutrophils in epithelium and no crypt destruction, erosions, ulcerations, or granulations.
Mucosal healing: both endoscopic healing and histologic healing.
Normal or inactive mucosal disease: Mayo endoscopy subscore of 0.
Symptomatic remission: Mayo stool frequency subscore of 0 or 1 and a rectal bleeding subscore of 0.
Normalization of CRP concentration: CRP concentration ≤3 mg/L.
Normalization of fecal lactoferrin concentration: fecal lactoferrin concentration ≤7.24 Ig/g.
Normalization of fecal calprotectin concentration: fecal calprotectin concentration 250 mg/kg.
Modified Mayo score response:
   Definition 1: a decrease in the modified Mayo score of ≥2 points and ≥35% and either a decrease in the rectal bleeding subscore of ≥1 or a rectal bleeding subscore of 0 or 1.
   Definition 2: a decrease in the modified Mayo score of ≥2 points and ≥30% and either a decrease in rectal bleeding of ≥1 or a rectal bleeding score of 0 or 1.

### Safety Results

Intravenous ustekinumab doses of both ˜6 mg/kg and 130 mg were generally well-tolerated with a safety profile that was generally comparable with placebo through Week 8. Of the 960 subjects in the safety analysis set, 1 or more treatment-emergent AEs was reported through Week 8 for 50.0%, 41.4%, and 48.0% of subjects in the ˜6 mg/kg, 130 mg, and placebo groups, respectively. Through Week 8, serious adverse effects (SAEs) were reported for 3.1%, 3.7%, and 6.6% of subjects in the ˜6 mg/kg, 130 mg, and placebo groups, respectively.

AEs within 1 hour of infusion were 0.9%, 2.2%, and 1.9% in the ˜6 mg/kg, 130 mg, and placebo groups, respectively.

The proportions of subjects with 1 or more infections were 15.3%, 15.9%, and 15.0% in the ˜6 mg/kg, 130 mg, and placebo groups, respectively. Serious infections were reported for 0.3%, 0.6%, and 1.3% of subjects in the ˜6 mg/kg, 130 mg, and placebo groups, respectively.

Pharmacokinetics Results

Serum samples were collected at Week 0 (preadministration), Week 0 (1 hr post-administration, Week 2, Week 4, and Week 8. For subjects randomized to ustekinumab treatment, a single IV infusion of ustekinumab was given either as a weight-based tiered dose of ˜6 mg/kg (ie, 260 mg for subjects with body-weight ≤55 kg, 390 mg for subjects with body-weight >55 kg and ≤85 kg, or 520 mg for subjects with body-weight >85 kg), or as a fixed dose of 130 mg. Considering that the median body-weight of subjects in the 130 mg group was 72 kg, the ustekinumab 130 mg dose corresponded to ˜2 mg/kg on a per-kg basis. Thus, on average, ustekinumab exposure in the 46 mg/kg group was approximately 3 times that of the 130 mg group. In line with this expectation, after a single IV administration of ustekinumab 46 mg/kg or 130 mg, median serum ustekinumab concentrations were approximately dose proportional at all sampling timepoints through Week 8. Median peak serum ustekinumab concentrations, which were observed 1 hour after the end of the infusion at Week 0, were 127.0 μg/mL and 43.16 μg/mL for the ˜6 mg/kg and 130 mg groups, respectively. At Week 8, the time of the primary efficacy endpoint, the median serum ustekinumab concentrations were 8.59 μg/mL and 2.51 μg/mL for the 46 mg/kg and 130 mg groups, respectively.

Subjects who were not in clinical response at Week 8 following administration of placebo IV at Week 0 received ustekinumab ˜6 mg/kg IV at Week 8, while subjects who were not in clinical response at Week 8 following administration of ustekinumab IV at Week 0 received ustekinumab 90 mg SC at Week 8. Among subjects who received placebo IV at Week 0 and who subsequently received ustekinumab 46 mg/kg IV at Week 8, median serum ustekinumab concentration at Week 16 (8 weeks after the ustekinumab IV dose) was slightly higher than that observed at Week 8 (among subjects who received ustekinumab 46 mg/kg IV at Week 0 [10.51 μg/mL versus 8.59 μg/mL, respectively]). Among subjects who received ustekinumab 90 mg SC at Week 8 (following their initial IV ustekinumab dose at Week 0), the median serum ustekinumab concentration at Week 16 was slightly higher in subjects who received ustekinumab 6 mg/kg IV at Week 0 compared to those who received ustekinumab 130 mg at Week 0 (1.92 μg/mL versus 1.59 μg/mL, respectively)

Immunogenicity Results

Of the 635 subjects in the ustekinumab groups with appropriate samples for the assessment of antibodies to ustekinumab, 4 (0.6%) subjects were positive for antibodies to ustekinumab through Week 8. Of these 4 subjects, 2 (50%) were positive for NAbs.

Of 822 subjects who received ustekinumab at any time through Week 16, and had appropriate samples for the assessment of anti-drug antibodies (ADAs), 18 subjects (2.2%) were positive for antibodies to ustekinumab through the final safety visit. Of these, 4 of 15 subjects (26.7%) were positive for NAbs among those evaluable for NAbs through the final safety visit. Among subjects who received ustekinumab 90 mg SC at Week 8, the incidence of antibodies to ustekinumab through Week 16 was numerically higher in the 130 mg IV→90 mg SC group compared to the ˜6 mg/kg IV→90 mg SC group (4.5% [6 of 132 subjects] vs 1.0% [1 of 101 subjects]).

Efficacy Results

Clinical Remission at Week 8—Global Definition

At Week 8, significantly greater proportions of subjects in the ˜6 mg/kg and 130 mg groups achieved clinical remission (15.5% and 15.6%, respectively) compared with subjects in the placebo group (5.3%; p<0.001 for both comparisons; Table 1).

TABLE 1
Number of Subjects in Clinical Remission (Global Definition) at Week 8

| | Placebo | Ustekinumab IV | | |
|---|---|---|---|---|
| | IV | 130 mg | 6 mg/kg | Combined |
| Primary Efficacy | 319 | 320 | 322 | 642 |
| Analysis Set | | | | |
| Week 8 (N) | 319 | 320 | 322 | 642 |
| Subjects in clinical | 17 | 50 | 50 | 100 |
| remission | (5.3%) | (15.6%) | (15.5%) | (15.6%) |
| Adjusted Treatment | | 10.3 | 10.2 | 10.2 |
| difference | | | | |
| (97.5% CI) | | (5.7, | (5.6, | (6.6, |
| | | 14.9) | 14.8) | 13.9) |
| p-value | | <0.001 | <0.001 | <0.001 |

N = number of subjects;
CI = confidence interval

Clinical Remission at Week 8—US Definition

At Week 8, significantly greater proportions of subjects in the ˜6 mg/kg and 130 mg groups achieved clinical remission (18.9% and 16.6%, respectively) compared with subjects in the placebo group (6.3%; p<0.001 for both comparisons; Table 2).

TABLE 2
Number of Subjects in Clinical Remission (US Definition) at Week 8

| | Placebo | Ustekinumab IV | | |
|---|---|---|---|---|
| | IV | 130 mg | 6 mg/kg | Combined |
| Primary Efficacy | 319 | 320 | 322 | 642 |
| Analysis Set | | | | |
| Week 8 (N) | 319 | 320 | 322 | 642 |
| Subjects in clinical | 20 | 53 | 61 | 114 |
| remission | (6.3%) | (16.6%) | (18.9%) | (17.8%) |
| Adjusted Treatment | | 10.3 | 12.7 | 11.5 |
| difference | | | | |
| (97.5% CI) | | (4.8, | (7.0, | (7.0, |
| | | 15.8) | 18.4) | 16) |
| p-value | | <0.001 | <0.001 | <0.001 |

N = number of subjects;
CI = confidence interval

Endoscopic Healing at Week 8

At Week 8, significantly greater proportions of subjects in the ˜6 mg/kg and 130 mg groups achieved endoscopic healing (27.0% and 26.3%, respectively) compared with subjects in the placebo group (13.8%; p<0.001 for both comparisons; Table 3).

TABLE 3
Number of Subjects with Endoscopic Healing at Week 8

| | Placebo | Ustekinumab IV | | |
|---|---|---|---|---|
| | IV | 130 mg | 6 mg/kg | Combined |
| Primary Efficacy | 319 | 320 | 322 | 642 |
| Analysis Set | | | | |
| Week 8 (N) | 319 | 320 | 322 | 642 |
| Subjects with endoscopic | 44 | 84 | 87 | 171 |
| healing | (13.8%) | (26.3%) | (27.0%) | (26.6%) |
| Adjusted Treatment | | 12.4 | 13.3 | 12.8 |
| difference | | | | |
| (95% CI) | | (6.5, | (7.3, | (7.9, |
| | | 18.4) | 19.3) | 17.8) |
| (97.5% CI) | | (5.2, | (6.4, | (7.2, |
| | | 19.2) | 20.1) | 18.5) |
| p-value | | <0.001 | <0.001 | <0.001 |

N = number of subjects;
CI = confidence interval

Clinical Response at Week 8

At Week 8, significantly greater proportions of subjects in the ˜6 mg/kg and 130 mg groups achieved clinical response (61.8% and 51.3%, respectively) compared with subjects in the placebo group (31.3%; p<0.001 for both comparisons; Table 4).

TABLE 4
Number of Subjects in Clinical Response

| | | Ustekinumab IV | | |
|---|---|---|---|---|
| | Placebo IV | 130 mg | 6 mg/kg | Combined |
| Primary Efficacy Analysis Set | 319 | 320 | 322 | 642 |
| Week 8 (N) | 319 | 320 | 322 | 642 |
| Subjects in clinical response | 100 (31.3%) | 164 (51.3%) | 199 (61.8%) | 363 (56.5%) |
| Adjusted Treatment difference | | 19.9 | 30.5 | 25.2 |
| (95% CI) | | (12.8, 27.3) | (23.2, 37.8) | (18.9, 31.5) |
| (97.5% CI) | | (11.4, 28.3) | (22.2, 38.8) | (18.0, 32.4) |
| p-value | | <0.001 | <0.001 | <0.001 |

N = number of subjects;
CI = confidence interval

Change in Baseline in Total IBDQ Score at Week 8

At baseline, median IBDQ scores were similar across all treatment groups. At Week 8, the median improvements from baseline in the IBDQ scores were significantly greater in the ˜6 mg/kg and 130 mg groups (31.0 and 31.5, respectively) compared with the placebo group (10.0; p<0.001 for both comparisons).

Clinical Remission at Week 8

When remission was assessed as clinical remission (global definition) with a rectal bleeding subscore of 0 at Week 8, the proportions of subjects who achieved this endpoint were almost identical to that observed based on the primary efficacy analysis (global definition). Significantly greater proportions of subjects in the ˜6 mg/kg and 130 mg groups achieved this endpoint (15.2% and 15.3%, respectively) compared with subjects in the placebo group (5.3%; p<0.001 for both comparisons).

Symptomatic Remission at Week 8

At Week 8, significantly greater proportions of subjects in the ˜6 mg/kg and 130 mg groups achieved symptomatic remission (44.7% and 41.3%, respectively) compared with subjects in the placebo group (22.6%; p<0.001 for both comparisons).

Histologic Healing at Week 8

Histologic healing was defined as 0 to <5% neutrophils in epithelium and no crypt destruction, erosions, ulcerations, or granulations. At Week 8, significantly greater proportions of subjects in the ˜6 mg/kg and 130 mg groups achieved histologic healing (35.6% and 37.9%, respectively) compared with subjects in the placebo group (21.9%; p<0.001 for both comparisons).

Change from Baseline in Mayo Score at Week 8

At baseline, the mean Mayo scores were the same across all treatment groups (8.9 for all groups). At Week 8, the mean decreases from baseline in Mayo scores were significantly greater in the ˜6 mg/kg and 130 mg groups (3.5 and 3.2, respectively) compared with the placebo group (1.8; p<0.001 for both comparisons).

Change from Baseline in Partial Mayo Score Through Week 8

At baseline, the mean partial Mayo scores were the same across all treatment groups (6.2 for all groups). As early as Week 2 and continuing for visits through Week 8, the mean decreases in the partial Mayo score were significantly greater in the ˜6 mg/kg and 130 mg groups compared with the placebo group. At Week 2, the mean decreases from baseline in the partial Mayo scores were 1.6 and 1.5, in the ˜6 mg/kg and 130 mg, respectively, compared with 1.0 in the placebo group (p<0.001 for both comparisons). At Week 8, the mean decreases from baseline in the partial Mayo scores were 2.9 and 2.6, in the ˜6 mg/kg and 130 mg, respectively, compared with 1.5 in the placebo group (p<0.001 for both comparisons).

UCEIS Score at Week 8

The UCEIS score provides an overall assessment of endoscopic severity of UC, based on mucosal vascular pattern, bleeding, and ulceration. The score ranges from 3 to 11 with a higher score indicating more severe disease by endoscopy. The UCEIS score was assessed only during the central read of the video of the endoscopy.

At baseline, the mean UCEIS scores were similar across all treatment groups (7.6, 7.5, 7.5 in the ˜6 mg/kg, 130 mg and placebo groups, respectively). At Week 8, the mean decreases from baseline in UCEIS scores were significantly greater in the ˜6 mg/kg and 130 mg groups (1.3 and 1.1, respectively) compared with the placebo group (0.5; p<0.001 for both comparisons).

At Week 8, significantly greater proportions of subjects in the ˜6 mg/kg and 130 mg groups had a UCEIS score of ≤4 (20.2% and 19.1%, respectively) compared with subjects in the placebo group (11.0%; p<0.001 and p=0.004, respectively). It is hypothesized that a UCEIS score of 4 is associated with Mayo endoscopic subscores of 0 or 1 that have defined endoscopic healing in this study.

Bristol Stool Form Scale Score

The BSFS score at a visit was the average of the 3-day daily average of the BSFS score prior to the visit. The same 3 days used to calculate the stool frequency and rectal bleeding subscores of the Mayo score were used to calculate the average BSFS score for the visit.

Approximately 40% (370/961) of randomized subjects had BSFS score collected at baseline. At baseline, 99.2% (367/370) of the subjects had average BSFS scores of ≥3 and the majority of subjects (54.3%) had average BSFS scores of ≥6, indicating diarrhea. As early as Week 2 and continuing for visits through Week 8, the proportions of subjects with diarrhea (average BSFS scores of ≥6) were smaller in the ˜6 mg/kg and 130 mg groups compared with the placebo group. At Week 8, 22.8%, 21.1%, and 32.0% of subjects had diarrhea (average BSFS scores of ≥6) in the ˜6 mg/kg, 130 mg and placebo groups, respectively. Furthermore, at Week 8 the proportion of subjects with normal stool (≥3 and <5) was greater in the ˜6 mg/kg and 130 mg groups compared with placebo (48.3%, 48.9%, and 29.3%, respectively).

Normalization of C-Reactive Protein

C-reactive protein (CRP) is used as a marker of inflammation in subjects with IBD. In UC, elevated CRP has been associated with severe clinical activity, an elevated sedimentation rate, and active disease as detected by colonoscopy. C-reactive protein was assayed using a validated, high-sensitivity CRP assay.

At baseline, the proportion of subjects who had abnormal CRP (>3 mg/L) was similar across all treatment groups; overall, 59.2% of randomized subjects had abnormal CRP concentrations at baseline. As early as Week 2 and continuing for visits through Week 8, among subjects who had abnormal values at baseline, significantly greater proportions of subjects in the ˜6 mg/kg and 130 mg groups achieved normalization of CRP (≤3 mg/L) compared with the placebo group. At Week 8, 38.7% and 34.1% of subjects achieved normalization of CRP in the ˜6 mg/kg and 130 mg groups, respectively, compared with 21.1% of subjects in the placebo group (p<0.001 for both comparisons).

Normalization of Fecal Lactoferrin

At baseline, the proportions of subjects with abnormal fecal lactoferrin (>7.24 μg/g) were similar across all treatment groups; overall 90.0% of randomized subjects had abnormal fecal lactoferrin concentrations at baseline. At Week 4 and Week 8, among subjects who had abnormal values at baseline, significantly greater proportions of subjects in the ˜6 mg/kg and 130 mg groups achieved normalization of fecal lactoferrin (≤7.24 μg/g) compared with the placebo group. At Week 8, 14.6% and 17.2% of subjects in the ˜6 mg/kg and 130 mg groups, respectively, achieved normalization of fecal lactoferrin compared with 9.3% of subjects in the placebo group (p=0.042, p=0.006, respectively, for the ustekinumab groups).

Normalization of Fecal Calprotectin

At baseline, the proportions of subjects with abnormal fecal calprotectin (>250 mg/kg) were slightly greater in the ˜6 mg/kg group (85.1%) compared with the placebo group (78.4%); 82.5% of subjects in the 130 mg group had abnormal fecal calprotectin at baseline. At Week 2 and Week 4, among subjects who had abnormal values at baseline, significantly greater proportions of subjects in the ˜6 mg/kg and 130 mg groups achieved normalization of fecal calprotectin (≤250 mg/kg). At Week 8, among subjects with abnormal fecal calprotectin at baseline, the proportions of subjects with normalized fecal calprotectin, though not significant, were numerically greater in the ustekinumab ˜6 mg/kg and 130 mg groups (25.5% and 24.2%, respectively), compared with subjects in the placebo group (20.4%; p=0.148, p=0.301 for both comparisons, respectively).

### Example 2: Maintenance Study of Ustekinumab in the Treatment of Ulcerative Colitis in Humans

Methodology

In this randomized-withdrawal maintenance study, all subjects enrolled were to be responders to study agent administered in the induction study. Primary (randomized) population: Subjects who were in clinical response to IV ustekinumab following induction comprised the primary population in the maintenance study. This population included the following: subjects who were randomized to receive ustekinumab (ie, 130 mg IV or ˜6 mg/kg IV) at Week 0 of the induction study and were in clinical response at induction Week 8; and subjects who were randomized to receive placebo at Week 0 of the induction study and were not in clinical response at induction Week 8 but were in clinical response at induction Week 16 after receiving a dose of IV ustekinumab (˜6 mg/kg) at induction Week 8 (placebo→ustekinumab ˜6 mg/kg IV). These subjects were randomized in a 1:1:1 ratio at maintenance Week 0 to receive ustekinumab 90 mg SC every 8 weeks (q8w), ustekinumab 90 mg SC every 12 weeks (q12w), or placebo SC. Nonrandomized population: Additional subjects entering the maintenance study were not randomized in the primary population and received maintenance treatment in this study as follows: ustekinumab induction delayed responders (ie, subjects who were not in clinical response to IV ustekinumab at induction Week 8 but were in clinical response at induction Week 16 after receiving ustekinumab 90 mg SC at induction Week 8) received ustekinumab 90 mg SC q8w; and placebo induction responders (ie, subjects who were in clinical response to placebo IV induction) received placebo SC. Nonrandomized subjects were followed for both efficacy and safety but were not included in the key efficacy analyses.

All subjects received their assigned dose of SC study agent at the maintenance Week 0 visit. Thereafter, to maintain the blind, all subjects received study agent at all scheduled study agent administration visits. Subjects were assessed for clinical flare at every visit and, if loss of clinical response was confirmed, were eligible for rescue medication. The main portion of the maintenance study was through Week 44 and a long-term study extension will continue through Week 220.

### Number of Subjects (Planned and Analyzed):

783 subjects who completed the induction study and were in clinical response to induction study agent were enrolled in this maintenance study. The numbers of subjects in each treatment group at maintenance Week 0 were as follows:
Randomized (primary) population (523 subjects [327 subjects were planned]):
   176 subjects were randomized to ustekinumab 90 mg SC q8w.
   172 subjects were randomized to ustekinumab 90 mg SC q12w.
   175 subjects were randomized to placebo SC.
Nonrandomized population (260 subjects):
   157 subjects who were ustekinumab induction delayed responders (ie, were not in clinical response to ustekinumab at induction Week 8 but were in clinical response at induction Week 16) received ustekinumab 90 mg SC q8w.
   103 subjects who were in clinical response to placebo IV induction (placebo induction responders) received placebo SC.

### Diagnosis and Main Criteria for Inclusion:

All subjects enrolled into this randomized-withdrawal maintenance study were those with moderately to severely active UC who had an inadequate response or had failed to tolerate conventional therapy (ie, corticosteroids or immunomodulators) or biologic therapy (ie, a TNF antagonist and/or vedolizumab), and demonstrated a clinical response to study agent during the induction study. This included subjects who were in clinical response to IV ustekinumab, in clinical response to IV placebo, or in delayed clinical response to ustekinumab, and had not received a protocol-prohibited medication change during the induction study.

### Criteria for Evaluation:

Pharmacokinetics (PK): Serum ustekinumab concentration
Immunogenicity: Antibodies to ustekinumab
Pharmacodynamics (PD)/biomarkers: Serum biomarkers; fecal microbiome; RNA expression and histologic assessment of disease activity and healing in mucosal biopsies
Genetics and epigenetics: Whole blood deoxyribonucleic acid (DNA)
Efficacy: Mayo score and partial Mayo score, UC Endoscopic Index of Severity (UCEIS), CRP, fecal lactoferrin, and fecal calprotectin
Health-related Quality of Life: Inflammatory Bowel Disease Questionnaire (IBDQ), 36-item Short Form Health Survey (SF-36), EuroQoL-5D Health Questionnaire (EQ-5D)
Health economics: UC disease-related hospitalizations and surgeries; productivity Visual Analog Scale (VAS), and Work Productivity and Activity Impairment Questionnaire-General Health (WPAI-GH)
Safety: Adverse events (AEs), serious adverse events (SAEs), infections, injection site reactions, allergic reactions, hematology and chemistry parameters, vital signs, physical examinations, and early detection of tuberculosis

Endpoints
The primary endpoint was clinical remission at Week 44. The definition of clinical remission (as well as the testing procedure) is different for submissions in the US and outside the US to accommodate the global and US preferred definitions of clinical remission. Each definition of clinical remission was applied to all subjects in the primary efficacy analysis set.
   The global definition of the primary endpoint of clinical remission was defined as a Mayo score ≤2 points, with no individual subscore >1.
   The US definition of clinical remission was defined as an absolute stool number ≤3, a Mayo rectal bleeding subscore of 0, and a Mayo endoscopy subscore of 0 or 1.
The major secondary endpoints, listed in the order in which they were tested, were:
   Maintenance of clinical response through Week 44
   Endoscopic healing at Week 44
   Clinical remission and not receiving concomitant corticosteroids (corticosteroid-free clinical remission) at Week 44
   Maintenance of clinical remission through Week 44 among the subjects who had achieved clinical remission at maintenance baseline
For the 3rd and 4th major secondary endpoints, the global definition of clinical remission was used to support submissions for countries outside the US and the US definition of clinical remission was used to support the submission in the United States.
   Demographic and baseline disease characteristics were summarized based on the 961 subjects in the primary efficacy analysis set.

Analyses of multiplicity-controlled endpoints, except for the fourth major secondary endpoint related to maintenance of clinical remission, were conducted using a Cochran-Mantel-Haenszel (CMH) chi square test stratified by clinical remission (global definition) status at maintenance baseline (yes/no as determined by the IWRS) and induction treatment (placebo IV [I-0]→ustekinumab ˜6 mg/kg IV [I-8], ustekinumab 130 mg IV [I-0], or ustekinumab ˜6 mg/kg IV [I-0]). For the fourth major secondary endpoint (maintenance of clinical remission), a CMH chi-square test stratified by induction treatment was used.

Global and US-specific multiple testing procedures were prespecified to control the overall Type 1 error rate at the 0.05 level over the multiplicity-controlled endpoints in this study (Section 3.11.2.7.3). All statistical testing was performed at the 2-sided 0.05 significance level. Nominal p-values are presented.

Safety was assessed by summarizing the frequency and type of treatment-emergent adverse events (AEs), laboratory parameters (hematology and chemistry), and vital signs parameters. Safety summaries are provided separately for randomized subjects, nonrandomized subjects, and all treated subjects. Presentation of the safety data focuses on the randomized population.

### Results:

Study Population

A total of 783 subjects who completed the induction study and were in clinical response to induction study agent were enrolled in this maintenance study. Of these, 523 subjects were in the targeted primary population for the maintenance study and were randomized to receive a SC administration of ustekinumab or placebo at maintenance Week 0 (176, 172, and 175 subjects in the ustekinumab 90 mg SC q8w, ustekinumab 90 mg SC q12w, and placebo groups, respectively). The remaining 250 subjects were in the nonrandomized population, including 157 ustekinumab induction delayed responders (who received ustekinumab 90 mg SC q8w) and 103 placebo induction responders (who received placebo). All enrolled subjects who were assigned treatment at maintenance baseline received their study agent at that time.

Prior to Week 40 (last dosing visit of the maintenance study), 85 subjects (16.3%) in the primary population discontinued study agent. The proportion of subjects who discontinued study agent was greater in the placebo group (24.6%) than those in the ustekinumab q8w and q12w groups (10.2% and 14.0%, respectively). The most common reasons for discontinuation were lack of efficacy and an adverse event due to worsening of UC. Prior to Week 44, 29 subjects (5.5%) in the primary population terminated study participation; the most common reason for termination of study participation was withdrawal of consent.

Baseline clinical disease characteristics were representative of a population of subjects with moderately to severely active UC that was refractory to available therapies and were generally well-balanced across the 3 treatment groups. The median duration of disease was 6.05 years and the median baseline Mayo score was 9.0, with 86.9% and 13.1% presenting with moderate and severe UC, respectively. At induction baseline, 52.2% of subjects in the primary population of the maintenance study were taking corticosteroids, 26.6% were taking immunomodulatory drugs, and 70.7% were taking aminosalicylates. The majority of subjects (93.5%) had an inadequate response to, or were intolerant of, corticosteroids and/or 6-MP/AZA, or demonstrated corticosteroid dependence at induction baseline. Overall in the primary population, 47.6% of subjects had a history of documented biologic failure and 52.4% of subjects did not. Also, 47.2% had failed at least 1 anti-TNF whereas 13.4% had failed both an anti-TNF and vedolizumab, and 49.3% were naïve to biologic therapy; 2 subjects were biologic failures to only vedolizumab.

Efficacy Results

Ustekinumab maintenance therapy demonstrated efficacy in a population of subjects with moderately to severely active UC who had previously failed or were intolerant of conventional or biologic therapies, including TNF antagonists and/or vedolizumab, and were in clinical response 8 weeks after receiving a single dose of ustekinumab IV induction therapy.

Based on the pre-specified global and US-specific multiple testing procedures, statistical significance can be claimed for both ustekinumab dose regimens (90 mg q8w and 90 mg q12w) for the primary endpoint of clinical remission at Week 44 and the three major secondary endpoints of maintenance of clinical response through Week 44, endoscopic healing at Week 44, and corticosteroid-free clinical remission at Week 44. Additionally, statistical significance can be claimed for maintenance of clinical remission through Week 44 (among the subjects who had achieved clinical remission at maintenance baseline) for both ustekinumab doses based on the US-specific testing procedure, and for the ustekinumab q12w regimen based on the global testing procedure.
Clinical Efficacy in the Primary Population (ie, Subjects in Clinical Response 8 Weeks After Receiving Ustekinumab IV Induction Therapy)
   Primary Endpoint: Clinical Remission
      The proportions of subjects in clinical remission (based on the global definition) at Week 44 were significantly greater in the ustekinumab q8w group and ustekinumab q12w group (43.8% and 38.4%, respectively) compared with subjects in the placebo group (24.0%; p<0.001 and p=0.002, respectively).
      The proportions of subjects in clinical remission (based on the US-specific definition) at Week 44 were significantly greater in the ustekinumab q8w group and ustekinumab q12w group (42.6% and 39.5%, respectively) compared with subjects in the placebo group (24.6%; p<0.001 and p=0.002, respectively).
      The effect of ustekinumab on achieving clinical remission (based on both the global and US specific definitions) was generally consistent across subgroups (including subjects who were biologic failures and those who were not biologic failures as well as subjects who were receiving concomitant immunomodulators or corticosteroids at induction baseline and those who were not) and was robust to prespecified changes in data-handling rules.
   Major Secondary Endpoints: Maintenance of Clinical Response, Endoscopic Healing, Corticosteroid-Free Clinical Remission, and Maintenance of Clinical Remission
      The proportions of subjects who maintained clinical response through Week 44, achieved endoscopic healing, achieved corticosteroid-free remission (applying both global and US specific definitions of clinical remission) were significantly greater (p<0.01) in the ustekinumab q8w and q12w groups compared with that in the placebo group.
      The proportions of subjects who maintained clinical remission among the subjects who had achieved clinical remission at maintenance baseline was numerically greater for both the ustekinumab q8w and q12w groups compared with that in the placebo group (applying both the global and US specific definition of clinical remission). Statistical significance (p<0.01) was achieved for both comparisons of the q8w and q12w groups versus placebo using the US-specific definition of clinical remission; however, statistical significance was only achieved for the q12w group (p<0.01) compared to placebo using the global definition of clinical remission.
   Other Histologic, Mucosal, Clinical, and Endoscopic Endpoints
   The analyses summarized below were not adjusted for multiplicity. Statements of statistical significance are based on nominal p-values.
      The proportions of subjects who achieved histologic healing (ie, neutrophil infiltration in <5% of crypts, no crypt destruction, and no erosions, ulcerations, or granulation tissue) at Week 44 were significantly (p<0.001) greater in the ustekinumab q8w and q12w groups compared with the placebo group.
      The proportions of subjects who achieved mucosal healing (a combination of endoscopic healing and histologic healing) at Week 44 were significantly (p<0.01) greater in the ustekinumab q8w and q12w groups compared with the placebo group.
      Applying both global and US-specific definitions of clinical remission, the proportions of subjects achieving corticosteroid-free remission for at least 90 days prior to Week 44 was significantly greater (p<0.01) in the ustekinumab q8w and q12w groups compared with that in the placebo group. Furthermore, among subjects receiving corticosteroids at maintenance baseline, significantly greater proportions of subjects (p<0.05) were in clinical remission and not receiving concomitant corticosteroids for at least 90 days prior to Week 44 in the ustekinumab q8w and q12w groups compared with those in the placebo group.
      The efficacy of ustekinumab maintenance treatment was also demonstrated in clinical outcomes as measured by maintained improvement in the partial Mayo score, maintenance of symptomatic remission as well as maintenance of endoscopic healing. Further evidence of the efficacy of ustekinumab maintenance treatment was observed in partial Mayo remission and symptomatic remission over time as well as symptom control (stool frequency and rectal bleeding).
   Inflammatory Biomarkers
      Over time through Week 44, the ustekinumab treatment groups maintained their CRP, fecal lactoferrin, and fecal calprotectin concentration levels observed at maintenance baseline, whereas median CRP, fecal lactoferrin, and fecal calprotectin concentrations worsened (increased) in the placebo group.
      At Week 44, the proportion of subjects with normalized CRP, fecal calprotectin and fecal lactoferrin were generally significantly greater in the ustekinumab q8w and q12w groups compared with the placebo group.
   Clinical Endpoints by Biologic Failure Status
      For subjects with and subjects without a history of biologic failure, the proportions of subjects who achieved each of the primary and major secondary endpoints and mucosal healing were generally greater in the ustekinumab q8w and q12w groups compared with subjects in the placebo group.
      In some cases, where treatment effects were similar in the biologic non-failure and failure populations, there was a consistent trend in the biologic-failure subjects across endpoints that the treatment effect for the ustekinumab q8w group was greater than that for the ustekinumab q12w group. This trend was not observed in the biologic non-failure population.
   Efficacy Based on Inflammatory Biomarker Subgroups
      Among subjects with a higher inflammatory burden (elevated CRP and/or elevated fecal inflammatory markers) at either induction or maintenance baseline, while both dosages generally demonstrated efficacy compared to placebo, the efficacy of ustekinumab q8w seemed to be better across the range of clinical endpoints than the ustekinumab q12w group. However, in subjects with low inflammatory burden at baseline, the ustekinumab q8w and q12w groups demonstrated similar efficacy over the endpoints
   Health-Related Quality of Life
      Through Week 44, subjects in the ustekinumab q8w and q12w groups were generally able to maintain improvement in health-related quality of life as assessed using the IBDQ, SF 36 and EQ 5D instruments compared to subjects in the placebo group.
   Outcomes for the Ustekinumab 90 mg q8w Dose and Ustekinumab 90 mg q12w Dose
      While both the ustekinumab q8w and q12w groups demonstrated generally similar efficacy for the primary and major secondary endpoints, q8w was modestly better than q12w based on the following more objective and stringent measures of efficacy, including:
         Endoscopic and mucosal healing at Week 44
         Durable partial Mayo remission at Week 44
         Corticosteroid-free clinical remission as well as the elimination of corticosteroids for at least 90 days prior to Week 44 among subjects receiving corticosteroids at maintenance baseline
      Furthermore, when efficacy was examined over time (for the following endpoints), the q8w group showed greater efficacy than the q12w group:
         Mayo stool frequency and rectal bleeding subscores indicating inactive or mild disease (ie, subscores of 0 or 1), as well as an absolute stool number ≤3 over time through Week 44.
         Partial Mayo remission and symptomatic remission over time through Week 44
         Median changes from baseline in fecal lactoferrin and calprotectin concentrations over time through Week 44.
Efficacy in Ustekinumab Induction Delayed Responders
Subjects who were delayed responders to ustekinumab induction therapy were able to maintain clinical response and achieve clinical remission, endoscopic, histologic, and mucosal healing (a combination of endoscopic healing and histologic healing) while receiving ustekinumab 90 mg q8w.
Efficacy and Pharmacokinetics/Immunogenicity
   In general, during maintenance, a positive association was observed between serum ustekinumab concentration and the clinical efficacy outcomes of clinical remission and endoscopic healing. In addition, lower levels of inflammation, as measured by CRP, were observed in subjects with higher serum ustekinumab concentrations.
   Among subjects receiving maintenance ustekinumab, the development of antibodies to ustekinumab did not appear to have an impact on clinical efficacy as measured by multiple endpoints such as clinical remission, endoscopic healing, clinical response, and change from maintenance baseline in Mayo score; however, the interpretation of the data is limited by the small sample size.

Pharmacokinetic and Immunogenicity Results
Following maintenance treatment with ustekinumab 90 mg SC q8w or q12w, steady-state was reached at approximately 8 or 12 weeks after subjects began receiving ustekinumab 90 mg SC q8w, or ustekinumab 90 mg SC q12w maintenance dose regimens, respectively. Median steady state trough serum ustekinumab concentrations over time were approximately 3-fold greater the concentrations in the ustekinumab q8w group (2.69 μg/mL to 3.09 μg/mL) than in the q12w group (0.92 μg/mL to 1.19 μg/mL).
Following maintenance dose regimens of ustekinumab 90 mg SC q8w or q12w, serum ustekinumab concentrations were sustained through Week 44 in almost all subjects, with a smaller proportion of subjects with undetectable trough concentrations over time in the 90 mg q8w group (0.7% to 2.4%) compared to those in the 90 mg q12w group (4.9% to 7.1%). The median ustekinumab concentration in subjects in the placebo group was below detectable levels by Week 16.
The impact of the different ustekinumab IV induction doses on serum ustekinumab concentrations during maintenance continued to diminish over time, as expected.
Median trough serum ustekinumab concentrations tended to be lower in subjects with higher body weight.
Nonrandomized subjects in the ustekinumab induction delayed responders group tended to have lower serum ustekinumab concentrations over time compared to randomized subjects in the ustekinumab q8w group following SC administration of the same ustekinumab dose regimen of 90 mg q8w.
Among 680 treated subjects with appropriate samples for the assessment of antibodies to ustekinumab, 39 (5.7%) were positive for antibodies to ustekinumab through 52 weeks of treatment, the majority with antibody titers 1:800. Of the 39 treated subjects who were positive for antibodies to ustekinumab in this maintenance study, 11 (28.2%) were positive for neutralizing antibodies.
In all randomized treatment groups, median serum ustekinumab concentrations were lower over time in subjects who were positive for antibodies to ustekinumab compared with levels in subjects who were negative for antibodies to ustekinumab.

Safety Results

Subcutaneous maintenance regimens of ustekinumab 90 mg administered q12w or q8w through Week 44 were generally well tolerated and consistent with the known safety profile of ustekinumab.
AEs were reported in 77.3%, 69.2%, and 78.9% of subjects in the ustekinumab q8w, ustekinumab q12w, and placebo groups, respectively.
   Reasonably related AEs were reported in 26.1%, 17.4%, and 28.6% of subjects in the ustekinumab q8w, ustekinumab q12w, and placebo groups, respectively.
Infections (as identified by the investigator) were reported in 48.9%, 33.7%, and 46.3% of subjects in the ustekinumab q8w, ustekinumab q12w, and placebo groups, respectively.
   Infections requiring oral or parenteral antibiotic treatment were reported in 22.7%, 15.7%, and 19.4% of subjects in the ustekinumab q8w, ustekinumab q12w, and placebo groups, respectively.
Serious infections were infrequent among randomized subjects and were reported in 1.7%, 3.5%, and 2.3% in the ustekinumab q8w, ustekinumab q12w, and placebo groups, respectively. Opportunistic infections were identified in 3 subjects (all in the randomized population); cytomegalovirus colitis was diagnosed for 2 subjects in the ustekinumab q12w group and 1 subject was diagnosed with concurrent moderate AEs of ophthalmic and labial herpes. No cases of active TB were reported among ustekinumab-treated subjects through Week 44.
The proportion of randomized subjects with AEs leading to discontinuation of study agent was higher in the placebo group than in the q12w and q8w groups and the most frequent AEs leading to discontinuation in the placebo group was worsening UC.
Among all treated subjects, including delayed ustekinumab induction responders, the overall safety profile was consistent with that observed in the randomized population.
There was 1 death reported for a subject who was a delayed ustekinumab induction responder and was receiving ustekinumab q8w. The cause of death was attributed to acute respiratory failure that occurred during thyroid surgery for a multinodular goiter.
Among all treated subjects, 2 subjects (1 subject in the ustekinumab induction delayed-responders group [receiving ustekinumab q8w] and 1 subject randomized to the placebo group who had received ustekinumab IV during induction) reported serious major adverse cardiovascular events; both events were associated with perioperative complications.
Among all treated subjects, there were 6 subjects for whom malignancies were reported (5 ustekinumab-treated subjects and 1 placebo-only subject).
   Three ustekinumab-treated subjects reported non-melanoma skin cancers (NMSCs); all had either a prior history of azathioprine or 6-MP treatment and 2 were on concomitant immunomodulator therapy at the time of the diagnosis.
   Two ustekinumab-treated subjects were reported to have solid tumors; one subject with a papillary renal cell carcinoma (q12w) and one subject with colon cancer (q8w); both tumors were detected early during the subject's participation in this maintenance study.
There were no cases of anaphylaxis or delayed hypersensitivity reactions identified among ustekinumab treated subjects.
There were no notable differences in the proportions of subjects with post-baseline maximum toxicity Grade ≥1 chemistry and hematology laboratory between the placebo and respective ustekinumab groups. Grade 3 and Grade 4 chemistry and hematology laboratory values were infrequent.

Health Economics and Medical Resource Utilization Results
Through Week 44, fewer subjects in the combined ustekinumab group had a UC disease-related hospitalization or surgery compared with the placebo group.
At Week 44, change from maintenance baseline in productivity visual analog scores (VAS) demonstrated improvement in subjects in the ustekinumab treatment groups and worsening in subjects in the placebo group.
At Week 44, percentages within each of the 4 WPAI-GH domains were maintained from maintenance baseline for the ustekinumab treatment groups, with additional improvement observed in subjects in the ustekinumab q8w group for percent impairment while working due to health, percent overall work impairment due to health, and percent activity impairment due to health. For subjects in the placebo group, percentages for all 4 WPAI-GH domains worsened (ie, increased).

### Conclusions

The ustekinumab maintenance study provided consistent and definitive evidence that the ustekinumab 90 mg SC q12w and q8w dose regimens were both effective in adult subjects with moderately to severely active UC who had responded to a single IV ustekinumab induction dose.
   The efficacy of ustekinumab was observed in subjects who were biologic failures as well as those who failed conventional but not biologic therapy (ie, biology-naïve).
   Of note, while both doses of ustekinumab were effective, the q8w dose regimen demonstrated modestly better efficacy across several objective and/or more stringent endpoints (eg, endoscopic healing and durable partial Mayo remission) as well as in overtime analyses of symptomatic and partial Mayo remission.
Maintenance dosing with ustekinumab SC dose regimens of 90 mg q12w and 90 mg q8w was generally well-tolerated over 44 weeks in this population of adult subjects with moderate to severe ulcerative colitis.
The safety and efficacy data from this study support a positive benefit/risk profile for ustekinumab SC maintenance therapy.

### Example 3: Long Term Extension of Maintenance Study of Ustekinumab in the Treatment of Ulcerative Colitis

Protocol CNTO1275UCO3001; Phase 3 Long-Term Extension of the Maintenance Study

### Protocol No.: CNT01275UCO3001

Title of Study: A Phase 3, Randomized, Double-blind, Placebo-controlled, Parallel-group, Multicenter Study to Evaluate the Safety and Efficacy of Ustekinumab Induction and Maintenance Therapy in Subjects with Moderately to Severely Active Ulcerative Colitis

### Study Name: UNIFI

### EudraCT Number: 2014-005606-38

### NCT No.: NCT02407236

### Clinical Registry No.: CR106920

### Principal Investigator: Bruce Sands, MD, (Division of Gastroenterology, Icahn School of Medicine at Mount Sinai; New York, NY, USA).

Study Center(s): 201 sites in Asia, Eastern Europe, North America, Western Europe, Israel, Australia, and New Zealand.
Publication (Reference): Sands B E, Sandborn W J, Panaccione R, et al. Ustekinumab as Induction and Maintenance Therapy for Ulcerative Colitis. N Engl J Med. 2019; 381(13):1201-1214.
Study Period: 19 Aug. 2015 (Date first subject signed informed consent) to 12 Aug. 2019 (Date of last observation for last subject recorded as part of the database)

### Phase of Development: 3

Objectives: The objectives of the long-term study extension (LTE) were to assess the efficacy, safety, pharmacokinetics (PK), and immunogenicity of an additional year of treatment with ustekinumab in subjects with moderately to severely active ulcerative colitis (UC) who had completed the 44-week maintenance study and who, in the opinion of the investigator, would benefit from continued treatment.
Methodology: Subjects who completed the safety and efficacy evaluations at Week 44 of the maintenance study and who, in the opinion of the investigator, might benefit from continued treatment had the opportunity to participate in the LTE for an additional 3 years of treatment. Randomized population: The primary (randomized) population in the maintenance study comprised subjects who were in clinical response to IV ustekinumab following induction. Subjects were randomized at maintenance baseline to placebo SC, ustekinumab 90 mg SC every 12 weeks (q12w), or ustekinumab 90 mg SC every 8 weeks (q8w). Nonrandomized population: Additional subjects who entered the maintenance study included: subjects in clinical response to placebo IV induction who received placebo SC during maintenance (ie, placebo induction responder group), and subjects who were delayed responders to ustekinumab induction (ie, subjects who were not in clinical response to ustekinumab at induction Week 8 but were in clinical response at induction Week 16 after receiving a SC administration of ustekinumab at induction Week 8) and received ustekinumab 90 mg SC q8w during maintenance (ie, the ustekinumab induction delayed-responder group).
Subjects were to continue to receive the same treatment regimen during the LTE that they were receiving at Week 44 of the maintenance study (either placebo, ustekinumab 90 mg SC q12w, or ustekinumab 90 mg SC q8w), with the first dose in the LTE being administered at Week 48. During the LTE, all subjects were to be assessed for worsening of UC disease activity based on the clinical judgment of the investigator. Subjects in the primary population (ie, those who were randomized at maintenance Week 0) whose UC disease activity worsened were eligible for a single dose adjustment as follows: placebo SC→ustekinumab 90 mg SC q8w; ustekinumab 90 mg SC q12w→ustekinumab 90 mg SC q8w; ustekinumab 90 mg SC q8w→continue on ustekinumab 90 mg SC q8w (sham dose adjustment). The first visit at which a subject was considered for a dose adjustment was at Week 56. Subjects were allowed 1 dose adjustment during the LTE.
The study blind was maintained during the LTE until the last subject in the maintenance study completed the Week 44 visit evaluations and the Week 44 analyses were completed. Therefore, subjects continued to receive study agent at all monthly visits until that time. After the study was unblinded to the investigative sites, subjects receiving placebo were terminated from study participation, and subjects receiving ustekinumab continued to receive ustekinumab, but had their study visits scheduled to coincide with their dose regimen (either q8w or q12w, as appropriate for their dose regimen).
Number of Subjects (planned and analyzed): 588 subjects who completed the safety and efficacy evaluation at Week 44 and were thought, in the opinion of the investigator, to benefit from continued treatment were treated in the LTE.
The 399 subjects randomized at maintenance baseline who were treated during the LTE were as follows:
   Placebo SC: 115 subjects
   Ustekinumab 90 mg SC q12w: 141 subjects
   Ustekinumab 90 mg SC q8w: 143 subjects
A total of 32.6% (130 subjects) of the randomized population had a dose adjustment during the LTE as follows:
   Among subjects randomized to placebo, 46.1% (53 subjects) had a dose adjustment to a ustekinumab 90 mg SC q8w dose regimen
   Among subjects randomized to ustekinumab 90 mg SC q12w, 28.4% (40 subjects) had a dose adjustment to a ustekinumab 90 mg SC q8w regimen
   Among subjects randomized to ustekinumab 90 mg SC q8w, 25.9% (37 subjects) had a sham dose adjustment (continued on the same dose regimen)
The 189 nonrandomized subjects in maintenance who were treated during the LTE were as follows:
   Placebo SC: 73 subjects who were in clinical response to placebo IV induction (responders to placebo IV induction) continued to receive placebo SC throughout maintenance and during the LTE until study unblinding, when they were discontinued from the study.
   Ustekinumab 90 mg q8w: 116 subjects were ustekinumab induction delayed responders (ie, were not in clinical response to ustekinumab at induction Week 8 but were in clinical response at induction Week 16 after receiving a SC administration of ustekinumab at induction Week 8) and continued to receive ustekinumab 90 mg SC q8w throughout maintenance and into the LTE.
      Diagnosis and Main Criteria for Inclusion: Subjects who completed the safety and efficacy evaluations at Week 44 of the maintenance study and who, in the opinion of the investigator, might benefit from continued treatment had the opportunity to participate in the LTE for an additional 3 years of treatment.
      Test Product, Dose and Mode of Administration, Batch No.: Ustekinumab was supplied as sterile liquid for SC injection in a single-use prefilled syringe (PFS). Each single-use PFS contained 90 mg (1.0 mL fill of liquid; bulk lot numbers 14L012, 15K142, 16B012, 16H032, 16L012, 17B042, 17J012, 18B092, and FJZ02) ustekinumab in an aqueous medium of L-histidine, L-histidine monohydrochloride monohydrate, sucrose, and polysorbate 80 at pH 6.0. No preservatives were present.
      Reference Therapy, Dose and Mode of Administration, Batch No.: Placebo was supplied as a sterile liquid for SC injection at a fill volume of 1.0 mL in a single-use PFS (bulk lot numbers 15L042, 16L022, 17F042, and EJSSL). Each PFS contained L-histidine, sucrose, and polysorbate 80 at pH 6.0. Placebo administrations had the same appearance as the respective ustekinumab administrations.
      Duration of Treatment: The main portion of the maintenance study was through Week 44 and an LTE will continue through Week 220. Duration of treatment in this first portion of the LTE was 52 weeks (Maintenance Week 44 through Week 96).

### Study Evaluations:

Pharmacokinetics: Serum ustekinumab concentration
Immunogenicity: Antibodies to ustekinumab
Efficacy: partial Mayo score, C-reactive protein (CRP), fecal lactoferrin, fecal calprotectin, and corticosteroid use
Health-related Quality of Life: Inflammatory Bowel Disease Questionnaire (IBDQ), 36-item Short Form Health Survey (SF-36)
Health economics: UC disease-related hospitalizations and surgeries; productivity Visual Analog Scale (VAS), and Work Productivity and Activity Impairment Questionnaire-General Health (WPAI-GH)
Safety: AEs, serious adverse events (SAEs), infections, injection-site reactions, allergic reactions, hematology and chemistry parameters, vital signs, physical examinations, and early detection of tuberculosis (TB)

### Statistical Methods:

The primary intent of the efficacy analyses was to assess maintenance of clinical benefit from the end of the main study (Week 44) through Week 92. Demographic and baseline disease characteristics, PK, immunogenicity, efficacy and safety analyses were performed for subjects treated in the LTE (including both randomized and nonrandomized subjects). Descriptive statistics (eg, mean, median, standard deviation, interquartile range, minimum, and maximum) were used to summarize continuous variables. Counts and percentages were used to summarize categorical variables. No statistical comparisons were made between treatment groups.

### Results:

Data are primarily summarized from Week 44 through Week 96 of the LTE.

### Study Population

A total of 588 subjects who completed the safety and efficacy evaluations at Week 44 and were thought, in the opinion of the investigator, to benefit from continued treatment were treated in the LTE with their same treatment regimen that they were receiving at maintenance Week 44. Of these, 399 subjects were from the randomized population in maintenance (115, 141, and 143 subjects in the placebo, ustekinumab 90 mg SC q12w, and ustekinumab 90 mg SC q8w groups, respectively). The remaining 189 subjects were from the nonrandomized population, including 73 placebo induction responders (received placebo) and 116 ustekinumab induction delayed responders (who received ustekinumab 90 mg SC q8w).

Prior to Week 96, 71 subjects (17.8%) from the randomized population discontinued study agent. The most common reasons for discontinuation of study agent in the combined ustekinumab group were Adverse event due to worsening of UC (2.5% [7 subjects]) and Other (2.5% [7 subjects]; most were reported as withdrawal of consent). Among nonrandomized subjects, 5 subjects (4.3%) from the ustekinumab induction delayed-responder group discontinued study agent; the most common reason for discontinuation was Other (1.7% [2 subjects]; both were reported as withdrawal of consent).
The clinical disease characteristics at Week 44 for randomized subjects who were treated in the LTE were generally similar for the ustekinumab q12w and q8w groups and numerically higher (eg, Mayo score, CRP concentrations) or lower (eg, subjects in remission) in the placebo group, indicating greater disease activity for the subjects in the placebo group. The clinical disease characteristics at Week 44 among subjects in the ustekinumab induction delayed-responder group (received ustekinumab q8w during the LTE) compared with the clinical disease characteristics of randomized subjects from the ustekinumab q8w group were indicative of greater disease activity in subjects from the ustekinumab induction delayed-responder group (eg, lower number of subjects in remission for the clinical efficacy endpoints, higher levels of inflammatory biomarkers).
The majority (93.7%) of subjects randomized in maintenance who were treated in the LTE demonstrated either an inadequate response to, or were intolerant of, corticosteroids and/or 6-mercaptopurine/azathioprine, or demonstrated corticosteroid dependence at induction baseline. Of the subjects randomized in maintenance who were treated in the LTE, 55.9% had no documented history of biologic failure at induction baseline (53.1% were biologic naïve and 2.8% were biologic experienced but did not have documentation of biologic failures). A total of 44.1% of randomized subjects had a documented history of biologic failure; the proportion of subjects was lower in the ustekinumab q12w group (37.6%) compared with the ustekinumab q8w group (49.7%). The history of response to and tolerance of UC medications, and UC medication history among subjects in the ustekinumab induction delayed-responder group who were treated during the LTE were generally consistent with those of the randomized population from the ustekinumab q8w group.

### Pharmacokinetics and Immunogenicity Results

Following treatment with ustekinumab 90 mg SC q8w or q12w during the LTE, sustained levels of ustekinumab were observed through Week 92 that were generally consistent with serum ustekinumab levels observed for these treatment groups during the maintenance study.
The incidence of antibodies to ustekinumab was low through Week 96 of the LTE.
   Among 400 subjects who received ustekinumab during both induction and maintenance through Week 96 of the LTE, 22 subjects (5.5%) were positive for antibodies to ustekinumab through Week 96 with most of the subjects having antibody titers 1:800.
   Among 515 all-treated subjects who received at least 1 dose of ustekinumab during induction or maintenance through Week 96 of LTE, 34 subjects (6.6%) were positive for antibodies to ustekinumab through Week 96 of this study with most of the subjects having antibody titers 1:800.
      The incidence of antibodies to ustekinumab appeared higher in subjects randomized to placebo (who originally received 1 infusion of ustekinumab during induction) in this maintenance study, or those who needed dose adjustment from placebo or ustekinumab q12w during the LTE.
      Of the 34 all-treated subjects who were positive for antibodies to ustekinumab, 8 (23.5%) subjects were positive for neutralizing antibodies.

### Efficacy Results

The intent of the efficacy analyses in the LTE was to assess maintenance of clinical benefit from the end of the main study (Week 44) through Week 92. It is important to note that subjects entered the LTE based on investigator determination as to whether the subject would benefit from continuation of treatment. The placebo group represents a subpopulation of UC patients who either were long-term responders to ustekinumab induction therapy (ie, were re-randomized to placebo maintenance) or placebo induction responders with a longer latency of disease. For these reasons, and because placebo subjects were to terminate from study participation after study unblinding, a direct comparison of findings between treatment groups was considered to be confounded; therefore no statistical comparisons were performed.

### Randomized Subjects Treated in the LTE

From Week 44 through Week 92, the proportions of randomized subjects in the ustekinumab q12w and q8w groups in symptomatic remission and the proportions in partial Mayo remission were sustained.
   Sustained efficacy was similarly observed in the biologic-naïve, biologic nonfailure, and biologic-failure populations.
With continued ustekinumab treatment in the LTE, subjects were able to achieve symptomatic remission and partial Mayo remission in the absence of corticosteroids at Week 92.
Continued treatment with ustekinumab enabled patients to eliminate corticosteroids.
With continued ustekinumab treatment from Week 44 through Week 92:
   Reductions in partial Mayo score observed at maintenance baseline were sustained with continued ustekinumab treatment from Week 44 through Week 92; the majority of subjects achieved a Mayo rectal bleeding subscore of 0, a Mayo stool frequency subscore of 0 or 1, and an absolute stool number ≤3.
   The reductions in CRP, fecal lactoferrin, and fecal calprotectin observed at maintenance baseline were sustained from Week 44 through Week 92.
   Improvements in health-related quality of life (IBDQ and SF-36) observed at maintenance baseline were sustained from Week 44 through Week 92.
Some benefit of dose adjustment was observed among randomized subjects treated in the LTE who had a dose adjustment.

### Ustekinumab Induction Delayed Responders Treated in the LTE

Subjects were able to sustain symptomatic remission and partial mayo remission from Week 44 through Week 92, achieve corticosteroid-free remission at Week 92, sustain reduction in inflammatory biomarkers from Week 44 through Week 92, and sustain improvement in health-related quality of life from Week 44 through Week 92
Clinical benefits observed among these subjects was similar to that observed for randomized subjects treated with ustekinumab q8w in the LTE.

### Efficacy and Pharmacokinetics

In general, high proportions (≥80%) of subjects were in symptomatic remission and partial Mayo remission in each concentration quartile. Accordingly, no clear exposure-efficacy relationship was observed between serum ustekinumab concentration and these efficacy endpoints in this population of subjects who were considered to have benefited from maintenance treatment.

### Efficacy and Immunogenicity

The proportions of randomized subjects in remission at Week 92 were comparable between those who were positive and those who were negative for antibodies to ustekinumab.

### Safety Results

Among all treated subjects in the LTE, the overall safety profile from Week 44 through Week 96 was generally consistent with the known safety profile of ustekinumab.
The number of subjects reporting AEs was generally comparable for subjects treated with ustekinumab as compared with subjects treated with placebo. The Infections and infestations and the Gastrointestinal disorders system-organ classes (SOCs) had the highest incidence of subjects who reported AEs.
   The incidences of subjects reporting AEs in the Infections and infestations SOC per hundred subject-years were 43.29, 48.91, and 46.48 in the placebo, ustekinumab q12w, and ustekinumab q8w groups, respectively. Nasopharyngitis was the most frequently reported AE, with incidences of 14.93, 21.55, and 19.83 in the placebo, ustekinumab q12w, and ustekinumab q8w groups, respectively.
   The incidences of subjects reporting AEs in the Gastrointestinal disorders SOC per hundred subject-years were 55.23, 34.82, and 31.53 in the placebo, ustekinumab q12w, and ustekinumab q8w groups, respectively. Ulcerative colitis was the most frequently reported AE, with a greater incidence among subjects in the placebo group (35.08) compared with the ustekinumab q12w and q8w groups (14.09 and 15.60, respectively).
The number of all-treated subjects who discontinued study agent because of 1 or more AEs per hundred subject-years of follow-up was 7.46, 4.97, and 4.23 in the placebo, ustekinumab q12w, and ustekinumab q8w groups, respectively. Ulcerative colitis was the most frequently reported AE leading to discontinuation, reported in 7.46, 2.49, and 2.28 subjects per hundred subject years of follow-up.
One subject died. The subject had received 1 dose of ustekinumab after dose adjustment from placebo; the immediate cause of death was attributed to cardiac arrest and was deemed unrelated to ustekinumab treatment. Prior to cardiac arrest, the subject with multiple comorbidities reported cytomegalovirus colitis, worsening UC, and failure to thrive.
The number of subjects with at least one SAE per hundred subject-years of follow-up were 10.45 in the placebo group and 6.30 in the combined ustekinumab 90 mg SC treatment group, which was comparable for those subjects in the ustekinumab q12w (5.80) and q8w (6.50) groups. The highest incidences of SAEs per hundred subject-years were related to ulcerative colitis: 5.22 in the placebo group and 1.63 in the combined ustekinumab group.
The total number of subjects with 1 or more infections per hundred subject-years of follow up were 45.53 in the placebo group and 49.73 in the combined ustekinumab group, which was comparable for those subjects in the ustekinumab q12w (50.57) and q8w (50.71) groups. The most frequently reported treatment-emergent infections were nasopharyngitis (14.18 and 19.15) and upper respiratory tract infection (5.22 and 6.54) in the placebo and combined ustekinumab groups, respectively.
   The incidences of subjects with 1 or more infections requiring oral or parenteral antimicrobial therapy per hundred subject-years of follow-up were 18.66 in the placebo group and 24.98 in the combined ustekinumab group. The most frequently reported infections requiring oral or parenteral antimicrobial treatment were nasopharyngitis, bronchitis, sinusitis, and upper respiratory tract infection.
Serious infections were reported infrequently; the incidence of subjects with 1 or more serious infections per hundred subject-years of follow up were 2.24 in the placebo group and 2.33 in the combined ustekinumab group. No specific event was reported in more than 1 subject.
No cases of active TB were reported among ustekinumab-treated subjects.
Opportunistic infections were identified in 2 subjects. Cytomegalovirus colitis was diagnosed for 1 subject in the placebo group who had a dose adjustment and received a single dose of ustekinumab 90 mg; the subject subsequently died of a cardiac arrest. Listeria monocytogenes infection was diagnosed for 1 subject in the ustekinumab q8w group; this event was reported as resolved with sequelae.
The proportions of all-treated subjects with 1 or more injection-site reactions to ustekinumab was 2.2% (n=10) and 0.9% (n=4) subjects reported injection-site reactions to placebo. No relationship between the development of antibodies to ustekinumab and injection-site reactions was identified in this study.
There were no cases of anaphylaxis or delayed hypersensitivity reactions identified among ustekinumab-treated subjects.
The number of treatment-emergent malignancies per hundred subject-years of follow-up was 1.49 in the placebo group (1 subject each: lentigo malignant melanoma and basal cell carcinoma [BCC]) and 0.93 in the combined ustekinumab group and was comparable between subjects in the q12w (0.83; 1 subject with BCC) and q8w (0.98; 2 subjects with BCC) groups. One additional subject (ustekinumab q8w group) entered the LTE but was not treated following a diagnosis of melanoma.
Among all treated subjects, serious major adverse cardiovascular events (MACE; 1 fatal) were reported in 3 subjects (2 subjects from the randomized placebo group who had received ustekinumab IV during induction and underwent dose adjustment to ustekinumab during the LTE, and 1 subject in the ustekinumab induction delayed responder group [receiving ustekinumab q8w]). Each of the subjects presented with confounding comorbidities at the time of the events.
There were no notable differences in the proportions of subjects with postbaseline maximum toxicity Grade ≥1 chemistry and hematology laboratory between the placebo and respective ustekinumab groups. Grade 3 and Grade 4 chemistry and hematology laboratory values were infrequent.
   Overall, the safety profile for randomized subjects was consistent with that observed among all treated subjects. Among the limited number of subjects who had a dose adjustment to ustekinumab q8w, the safety profile was generally consistent with that observed in subjects randomized in maintenance to ustekinumab q8w. Serious adverse events and AEs leading to discontinuation of study agent were infrequent events among subjects who had a dose adjustment to ustekinumab q8w, with ulcerative colitis as generally the most frequently reported event.
   The overall safety profile of ustekinumab for subjects who were delayed responders and were treated in the LTE was consistent with that observed in the randomized ustekinumab q8w group.

### Health Economics and Medical Resource Utilization Results

Among randomized subjects treated in the LTE:
The proportion of subjects with a UC disease-related hospitalization or surgery from Week 0 of induction through Week 96 was low in both the placebo group (4.3% [5 subjects]; subjects in this group received a single IV dose of ustekinumab during induction) and the combined ustekinumab group (3.9% [11 subjects]).
At Week 92, the improvements in productivity VAS score observed at maintenance baseline among ustekinumab treatment groups were maintained.
At Week 92, the WPAI-GH mean percentages were maintained from maintenance baseline for the ustekinumab q12w and q8w groups in all 4 WPAI domains, with additional improvement (ie, decrease) observed in subjects in both ustekinumab groups for percent impairment while working due to health, percent overall work impairment due to health, and percent activity impairment due to health.

### Study Limitations

Subjects were selected by the investigator to participate in the study LTE because, in their opinion, they might benefit from continued treatment. This criterion may limit the generalizability of the findings to only those who responded to and tolerated ustekinumab in the first year of treatment.
Subjects could change concomitant medications at any time during the LTE to mimic real world practice.
Direct efficacy comparisons between placebo and ustekinumab treatment groups were not performed since subjects who entered the study LTE on placebo represent a group of patients who were long-term responders to ustekinumab induction or were true placebo responders. In addition, placebo subjects were discontinued from the study when study unblinding occurred, limiting the value of direct comparisons between the placebo and ustekinumab treatment groups. As a result, the emphasis of clinical outcomes reported was on efficacy measures among ustekinumab-treated subjects.
The decision to dose adjust was based on the clinical judgement of the investigator regarding a subject's disease activity; no protocol-specified criteria (eg, clinical flare based on partial Mayo score) were applied, and some subjects were in remission at the time of the dose adjustment, thereby limiting the interpretability of these data.
Clinical outcomes in subpopulations based on biologic-failure status (ie, biologic naïve, biologic nonfailure, and biologic failure) are presented for the purpose of evaluating the consistency of outcomes in these populations with those in the overall population; however, due to the limited sample sizes in these analyses, these results should also be interpreted with caution.

### Conclusions

Treatment with ustekinumab 90 mg SC q12w and q8w maintained remission measured as either symptomatic remission or partial Mayo remission through the second year of treatment.
Maintenance of efficacy through a second year of treatment was supported by sustained reductions in inflammatory markers of disease and sustained improvement in health-related quality of life measures.
No new safety signals were identified in the second year of maintenance therapy.
   The safety profile is consistent with previously reported safety data through the first year of treatment in UC and with the overall ustekinumab safety profile.

### List of Abbreviations and Definitions of Terms

6-MP 6-mercaptopurine
AE adverse event
ALT alanine aminotransferase
AST aspartate aminotransferase
AZA azathioprine
BCC basal cell carcinoma
CMV cytomegalovirus
CRP C-reactive protein
CSR clinical study report
DBL database lock
DMC Data Monitoring Committee
ECG electrocardiogram
eCRF electronic case report form
IBDQ Inflammatory Bowel Disease Questionnaire
ITT intent to treat
IWRS interactive web response system
LLT lower-level term
LTE long-term (study) extension
MACE major adverse cardiovascular event(s)
MCS mental component summary
MedDRA Medical Dictionary for Regulatory Activities
NAb neutralizing antibody
NCI-CTCAE National Cancer Institute Common Terminology Criteria for Adverse Events
NMSC nonmelanoma skin cancer
PCS physical component summary
P.Eq. prednisone equivalent
PFS prefilled syringe
PK pharmacokinetic(s)
PT preferred term
q8w every 8 weeks
q12w every 12 weeks
SAE serious adverse event
SCC squamous cell carcinoma
SF-36 36-item Short Form Health Survey
SOC system-organ class
TB tuberculosis
TNF tumor necrosis factor
UC ulcerative colitis
VAS visual analog scale
WPAI-GH Work Productivity and Activity Impairment Questionnaire-General Health

### 1. Introduction

The Phase 3 development program for ustekinumab in the treatment of ulcerative colitis (UC) consists of 2 separate studies—an induction study and a maintenance study—conducted under the same protocol (CNTO1275UCO3001). Both studies are Phase 3, randomized, double-blind, placebo-controlled, parallel-group, multicenter studies of ustekinumab in subjects 18 years or older with moderately to severely active UC.
The induction study targeted subjects who demonstrated an inadequate response or failure to tolerate conventional or biologic therapy.
Subjects who achieved clinical response to IV ustekinumab at Week 8 or Week 16 of the induction study were eligible for entry into the randomized-withdrawal maintenance study evaluating the safety and efficacy of SC ustekinumab maintenance treatment through 44 weeks. Scope of the 96-Week Clinical Study Report
This CNTO1275UCO3001 96-week CSR summarizes the efficacy, safety, pharmacokinetics (PK), and immunogenicity results from Week 44 through Week 96 for subjects who continued into the long-term extension (LTE) of the maintenance study.

### 2. Objectives

The objectives of the study LTE were to assess the efficacy, safety, PK, and immunogenicity of an additional 3 years of treatment with ustekinumab in subjects with moderately to severely active UC who had completed the maintenance study through Week 44 and who, in the opinion of the investigator, would benefit from continued treatment.

### 3. Methods

### 3.1. Overview of Phase 3 Program Design

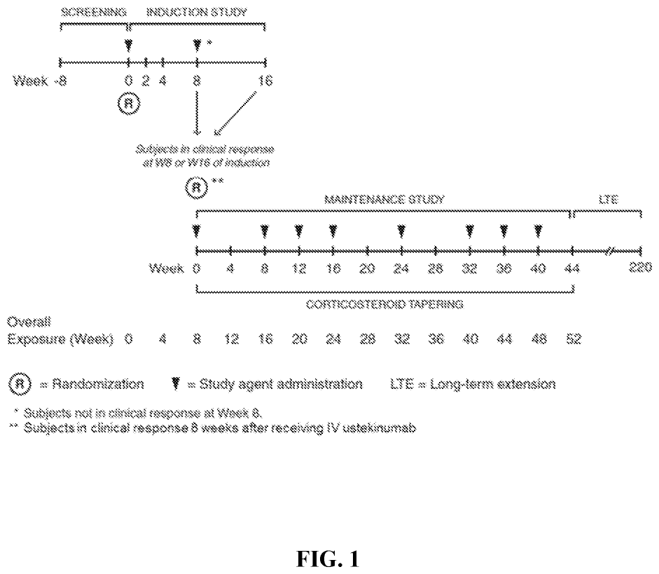

The induction and maintenance studies were Phase 3, randomized, double-blind, placebo-controlled, parallel-group, multicenter studies of ustekinumab in subjects 18 years or older with moderately to severely active UC conducted under a single protocol. The induction study targeted subjects who demonstrated an inadequate response or failure to tolerate conventional or biologic therapy (i.e., a tumor necrosis factor [TNF] antagonist and/or the integrin antagonist, vedolizumab). The maintenance study was a randomized-withdrawal study that targeted subjects who demonstrated a clinical response to induction treatment with IV ustekinumab. After completion of the maintenance study (i.e., through Week 44), eligible subjects were to be followed for an additional 3 years of treatment in an LTE also conducted under this protocol. A diagrammatic representation of the study design is presented in FIG. 1.

### Maintenance Study Design

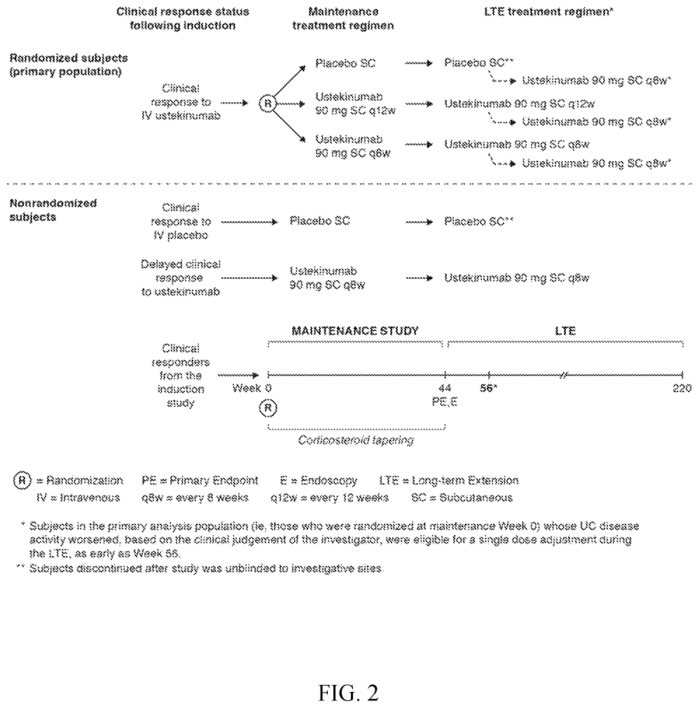

In the maintenance study, all subjects enrolled were to be responders to study agent administered in the induction study. The schema for the maintenance study is shown in FIG. 2.
The primary population in the maintenance study comprised subjects who were in clinical response to IV ustekinumab following induction. This population included the following:
Subjects who were randomized to receive ustekinumab (i.e., 130 mg IV or ˜6 mg/kg IV) at Week 0 of the induction study and were in clinical response at induction Week 8.
Subjects who were randomized to receive placebo at Week 0 of the induction study and were not in clinical response at induction Week 8 but were in clinical response at induction Week 16 after receiving a dose of IV ustekinumab (˜6 mg/kg) at induction Week 8 (placebo→ustekinumab ˜6 mg/kg IV).\
Subjects who were in clinical response to ustekinumab IV induction were randomized in a 1:1:1 ratio to 1 of 3 treatment groups (Table 6) at the Week 0/baseline visit of the maintenance study:
   Placebo SC
   Ustekinumab 90 mg SC every 12 weeks (q12w)
   Ustekinumab 90 mg SC every 8 weeks (q8w)
      Eligible subjects were allocated to a treatment group using a permuted block randomization with clinical remission (defined as a Mayo score ≤2 points, with no individual subscore >1) status at maintenance baseline (yes/no), oral corticosteroid use at maintenance baseline (yes/no), and induction treatment (placebo IV [induction Week 0]→ustekinumab ˜6 mg/kg IV [induction Week 8], ustekinumab 130 mg IV [induction Week 0], or ustekinumab ˜6 mg/kg IV [induction Week 0]) as stratification variables.
      Additional subjects entering the maintenance study included the following; these subjects were not randomized and are not part of the primary population:
Subjects who were in clinical response to placebo IV induction received placebo SC (ie, the placebo induction responder group)
Subjects who were delayed responders to ustekinumab induction (ie, were not in clinical response to ustekinumab at induction Week 8 but were in clinical response at induction Week 16 after receiving a SC administration of ustekinumab at induction Week 8) received ustekinumab 90 mg SC q8w (ie, the ustekinumab induction delayed-responder group)
   All subjects received their assigned dose of SC study agent at the maintenance Week 0 visit. The maintenance study continued to Week 44.

### Long-Term Extension Study Design

Subjects who completed the safety and efficacy evaluations at Week 44 and who, in the opinion of the investigator, might benefit from continued treatment had the opportunity to participate in the LTE The LTE began after the assessments listed for the maintenance Week 44 visit (M-44) were completed and will continue through Week 220.
Subjects were to continue to receive the same treatment regimen during the LTE that they were receiving at the end of the maintenance study (either placebo, ustekinumab 90 mg SC q12w, or ustekinumab 90 mg SC q8w), with the first dose in the LTE being administered at Week 48.
During the LTE, all subjects were to be assessed for worsening of UC disease activity based on the clinical judgment of the investigator. Subjects in the primary population (i.e., those who were randomized at maintenance Week 0) whose UC disease activity worsened were eligible for a single dose adjustment as follows:
Placebo SC→ustekinumab 90 mg SC q8w
Ustekinumab 90 mg SC q12w→ustekinumab 90 mg SC q8w
Ustekinumab 90 mg SC q8w→continue on ustekinumab 90 mg SC q8w
   The first visit at which a subject was considered for a dose adjustment was at Week 56. Subjects were allowed 1 dose adjustment during the LTE.
   The interactive web response system (IWRS) ensured that SC ustekinumab was not administered more frequently than q8w. For example, subjects randomized to the ustekinumab 90 mg SC q12w group whose disease activity was identified as worsening by the investigator were to receive ustekinumab 90 mg SC at the current visit only if the last dose of ustekinumab was administered at least 8 weeks before this visit. If the last administration of ustekinumab 90 mg SC was less than 8 weeks before, the next administration of ustekinumab 90 mg SC was to be initiated at the next scheduled visit that occurred at least 8 weeks after the previous administration of ustekinumab. Starting at Week 56, the investigator was directed per protocol to assess for potential worsening of a subject's UC disease activity and, in their clinical opinion, a need for dose adjustment if the subject had not had a dose adjustment; the site entered “yes” or “no” to a question of whether the subject required a dose adjustment. If “yes” the IWRS managed dose adjustment and the distribution of study agent in a blinded manner until the study was unblinded. Following study unblinding, subjects receiving ustekinumab q12w could have a dose adjustment to ustekinumab q8w if they had not yet had one.
   Subjects who were not in the primary population (i.e., placebo induction responders, ustekinumab induction delayed responders) were not eligible for a dose adjustment during the LTE.
   Any subject who, in the opinion of the investigator, did not show improvement in his or her UC disease activity by 16 weeks after dose adjustment was to be discontinued from further study agent administration.
   During the LTE, all concomitant medications, including UC-specific medications (with the exception of certain prohibited medications listed below), were allowed to be administered at the discretion of the investigator.
   Efficacy evaluations during the LTE include the partial Mayo score, markers of inflammation, and corticosteroid use. The full Mayo score (including an endoscopy) is to be assessed at the final efficacy visit at Week 200, at the time of study agent discontinuation, or at the time of study participation termination. Selected patient-reported outcomes and health economics data were also collected. Safety evaluations include an assessment of adverse events (AEs) and routine laboratory analyses, with a final safety visit at Week 220 or approximately 20 weeks after a subject's last administration of study agent (for subjects who have not terminated study participation). All study evaluations performed during the LTE are listed in the Time and Events Schedule of the protocol.
The study blind was maintained during the LTE until the last subject in the maintenance study completed the Week 44 (M-44) visit evaluations and the Week 44 analyses were completed. Therefore, subjects continued to receive study agent at all monthly visits until that time. After the study was unblinded to the investigative sites, subjects receiving placebo were terminated from study participation, and subjects receiving ustekinumab continued to receive ustekinumab, but had their study visits scheduled to coincide with their dose regimen (either q8w or q12w, as appropriate for their dose regimen).
   The sponsor was blinded to treatment assignment in the maintenance study until after the Week 44 DBL occurred. To minimize bias and protect the integrity of the clinical program, treatment assignment blinding was maintained (for both the induction and maintenance studies) for investigative sites, site monitors, and subjects participating in this protocol until the Week 44 analyses were completed. Subjects entered the LTE at their assigned maintenance dose regimens (eg, q8w or q12w) receiving injections of study agent every 4 weeks (except for Week 52) to maintain the blind, with the first injection administered at Week 48. After the study was unblinded to the investigative sites, subjects receiving placebo were terminated from study participation and subjects receiving ustekinumab continued to receive ustekinumab, but had their study visits scheduled to coincide with their dose regimen (q8w or q12w, as appropriate, through Week 200).
   During the LTE, all concomitant medications, including UC-specific medications (with the exception of prohibited medications listed below), were allowed to be administered at the discretion of the investigator. The prohibited therapies were also not to be used as rescue medications.

### Study Evaluations

The following study evaluations were conducted:
Pharmacokinetics: serum ustekinumab concentration
Immunogenicity: antibodies to ustekinumab
Efficacy: partial Mayo score, C-reactive protein (CRP), fecal lactoferrin, fecal calprotectin, and corticosteroid use
Health-related quality of life: Inflammatory Bowel Disease Questionnaire (IBDQ), 36-item Short Form Health Survey (SF-36)
Health economics: UC disease-related hospitalizations and surgeries; productivity Visual Analog Scale (VAS), and Work Productivity and Activity Impairment Questionnaire-General Health (WPAI-GH)
Safety: AEs, serious adverse events (SAEs), infections, injection-site reactions, allergic reactions, hematology and chemistry parameters, vital signs, physical examinations, and early detection of tuberculosis (TB)

### Pharmacokinetics and Immunogenicity

Blood samples for determining the serum ustekinumab concentrations and immunogenicity of ustekinumab (antibodies to ustekinumab) were collected from all subjects as indicated in the LTE Time and Events Schedule. Analyses were performed as previously presented in the UCO3001 44W.

### Efficacy Evaluations

Efficacy evaluations through Week 92 for those subjects who entered the LTE were performed as indicated in the LTE Time and Events Schedule and included the partial Mayo score, CRP, fecal lactoferrin and fecal calprotectin, corticosteroid use, IBDQ, and SF-36.
Descriptions of the individual efficacy assessments were previously presented in the UCO3001 44W.

### Efficacy Criteria

Efficacy endpoints were defined as follows:
Clinical remission: Mayo score ≤2 points, with no individual subscore >1
Symptomatic remission: Mayo stool frequency subscore of 0 or 1 and a rectal bleeding subscore of 0
Partial Mayo remission: partial Mayo score ≤2
IBDQ remission: IBDQ ≥170
Normalization of CRP concentration: CRP concentration 3 mg/L
Normalization of fecal lactoferrin concentration: fecal lactoferrin concentration ≤7.24 μg/g
Normalization of fecal calprotectin concentration: fecal calprotectin concentration ≤250 mg/kg

### Safety Evaluations

Safety through Week 96 was evaluated based on AEs and clinical laboratory test results (i.e., hematology and serum chemistry) as previously described in the UC03001 44W. With the exception of clinical laboratory data, the data for safety variables were recorded on or appended to the electronic case report forms. Clinical laboratory data were collected and saved in an electronic file format. The timing of all safety procedures was described in the LTE Time and Events Schedule in the protocol.
Safety data obtained during the study were reviewed on a routine basis by an unblinded, independent DMC until the Week 44 DBL.

### Health Economics and Medical Resource Utilization

Medical resource utilization data, including UC-related hospitalizations and UC-related surgeries, were collected. Additionally, the potential impact of ustekinumab on subjects' work limitations and daily productivity was assessed through the WPAI-GH and the productivity VAS, respectively.

### Data Quality Assurance

The study was monitored according to the Sponsor's current standard operating procedure for the monitoring of clinical trials.

### Statistical Methods

The objective of the study LTE was primarily to enable subjects reaching Week 44 of the maintenance study to continue to receive study agent without interruption. The primary intent of this study report is to assess efficacy from the end of the maintenance study (Week 44) through Week 92 (last efficacy assessment prior to Week 96) of the LTE and safety from the end of the maintenance study through Week 96 of the LTE, though the data before Week 44 were also included.
It is important to note that subjects entered the LTE based on investigator determination as to whether the subject would benefit from continuation of treatment. Furthermore, the placebo group represents a subpopulation of UC patients who either were long-term responders to ustekinumab induction therapy (i.e., were re-randomized to placebo maintenance) or placebo induction responders with a longer latency of disease. For these reasons, and because placebo subjects were to terminate from study participation after study unblinding, a direct comparison of findings between placebo and ustekinumab treatment groups was considered to be confounded; therefore, no statistical comparisons were performed.
Descriptive statistics (e.g., mean, median, standard deviation, interquartile range, minimum, and maximum) were used to summarize continuous variables. Counts and percentages were used to summarize categorical variables.

### Planned Analyses

Planned analyses for the LTE are described below.

### Populations for Analysis

### Efficacy

Efficacy summaries were provided for randomized subjects at Week 0 of the maintenance study who were treated in the LTE. Selected efficacy summaries were also provided for subjects who had a dose adjustment during the LTE and for nonrandomized subjects at Week 0 of the maintenance study who were treated in the LTE. In addition, efficacy summaries were provided, separately, for all randomized and nonrandomized subjects at maintenance baseline for the endpoints of symptomatic remission and partial Mayo remission, regardless of whether subjects were treated in the LTE.
The main population for efficacy summaries comprised randomized subjects who were treated in the LTE.

### Safety

Summaries of safety were based on all treated subjects who received at least 1 administration of study agent in the LTE. Additional summaries were also provided based on randomization status (i.e., randomized or nonrandomized in the maintenance study), up to the time of dose adjustment; and based on randomized subjects, including the data following dose adjustment.
Treated subjects in the LTE was the main population for safety summaries.

### Pharmacokinetics

Pharmacokinetic analyses were based on all subjects who received at least 1 administration of ustekinumab during the LTE, including both randomized and nonrandomized subjects. The analyses were also performed for subjects who had a dose adjustment during the LTE.

### Immunogenicity

Immunogenicity analyses were based on all subjects who were treated in the LTE and received at least 1 administration of ustekinumab, and had at least 1 sample obtained after their first dose of ustekinumab for detection of antibodies to ustekinumab.

### Pharmacokinetics

Serum concentrations at Week 44, Week 68, and Week 92 were summarized for each treatment.
All concentrations below the lowest quantifiable concentration were labeled as such in listings containing concentration data. Concentrations below the lowest quantifiable concentration were treated as zero in the summary statistics.
In serum ustekinumab concentration summaries that included data through Week 44, the following data were excluded, from the time of occurrence through Week 44: data collected for subjects who: (1) discontinued study agent, (2) skipped an injection, (3) received an incomplete injection, (4) received an incorrect injection, (5) received an additional injection, and/or (6) received commercial ustekinumab. In addition, PK samples taken outside the scheduled visit window (±10 days of each scheduled visit) were excluded from the summaries. These exclusion rules were not applied to data after Week 44.

### Immunogenicity

The incidence of antibodies to ustekinumab were summarized for subjects who were treated in the LTE through Week 96 and had appropriate samples for detection of antibodies to ustekinumab (ie, subjects with at least 1 sample obtained after their first dose of ustekinumab).
Serum ustekinumab concentrations at Week 44, Week 68 and Week 92 by antibody to ustekinumab status through Week 96 were summarized by treatment group based on randomized subjects in maintenance who received ustekinumab in the LTE.
A listing of subjects who were positive for antibodies to ustekinumab from induction Week 0 through Week 96 was provided.

### Efficacy

### Data-Handling Rules

Treatment Failure Rules: Unless otherwise mentioned, subjects who had an ostomy or colectomy, or discontinued study agent due to lack of therapeutic effect or due to an AE of worsening of UC or had a dose adjustment (only occurred from Week 56 onward) prior to the designated visit, were considered to be a treatment failure from the time of event onward.
For dichotomous endpoints, subjects who had a treatment failure were considered not to have achieved the respective endpoints from the time of treatment failure onwards. For continuous endpoints, subjects who had a treatment failure had their induction baseline values carried forward from the time of the treatment failure onwards.
Missing Data Rules: For subjects with missing data, unless otherwise specified, the last observation was carried forward for continuous endpoints, with the exception of the partial Mayo scores where the last available Mayo subscores were carried forward. For dichotomous endpoints, subjects with missing data were considered not to have achieved the respective endpoints Treatment failure rules overrode missing data rules. This means that if a subject had an event of treatment failure, induction baseline values were assigned from the point of treatment failure onward for continuous endpoints, and subjects were considered as not achieving the respective endpoints for dichotomous endpoints, regardless of whether the data were observed or missing.

### Analysis Approaches

This CSR adopted three analysis approaches for treated subjects in the LTE as described below:
As observed: Data were summarized through Week 92 or up to the time of dose adjustment with treatment failure rules applied, excluding subjects with missing data who had not had a treatment failure prior to the designated analysis timepoint.
Intent-to-treat (ITT): Data were summarized through Week 92 with treatment failure and missing data rules applied.
Dose adjustment as a treatment strategy: Similar to the corresponding ITT analysis approach except that the dose adjustment treatment failure criterion was suspended (i.e., subjects who had a dose adjustment were not considered to be a treatment failure). The rest of the analysis rules were kept the same.
   In the as-observed analysis approach, at each analysis time point, only those subjects who had data available or who had a treatment failure prior to that time point (considered as nonresponders) were included in the analysis. This approach was considered reasonable as only those patients with missing data not related to treatment failure (presumably missing at random) were excluded from the analysis.
   In the ITT analysis approach, the number of subjects included in the analysis was fixed over time. As it was expected that more subjects would undergo dose adjustment (a treatment failure criterion) or discontinue study agent (whether or not it would be due to lack of therapeutic effect or due to an AE of worsening of UC) over time, the proportion of subjects who achieved binary endpoints was expected to decrease over time. As such, the ITT analysis approach was considered conservative.
   The conservative ITT analysis approach was used as the default for efficacy analyses. However, analyses based on an as-observed analysis approach were performed for key efficacy endpoints such as symptomatic remission, partial Mayo remission and the change from baseline in partial Mayo score, and were considered to be more reasonably reflective of efficacy in the LTE.
   The dose-adjustment-as-a-treatment-strategy analysis approach was considered pragmatic as it reflects the clinical practice where treatments are optimized either through increases in dose or dosing frequency.

### Clinical Endpoints

A list of clinical endpoints summarized in this CSR along with the associated analysis population and analysis approach based on subjects treated in the LTE is provided in Table 5. Clinical remission referenced in the analyses was based on the global definition (Mayo score ≤2 points, with no individual subscore >1).
In addition to the summaries based on treated subjects in the LTE, symptomatic remission and partial Mayo remission were summarized, separately, for all randomized and nonrandomized subjects at maintenance baseline, based on the dose-adjustment-as-a-treatment-strategy (randomized subjects only) and ITT analysis approaches, regardless of whether subjects were treated in the LTE. In this type of analysis, consistent with the treatment failure rules applied in UCO3001 W44 CSR, subjects who had a prohibited change in UC medication, an ostomy or colectomy, or used a rescue medication after clinical flare, or discontinued study agent due to lack of therapeutic effect or due to an AE of worsening of UC prior to the Week 44 visit were considered to be a treatment failure prior to or at Week 44. After Week 44, subjects who had an ostomy or colectomy, or discontinued study agent due to lack of therapeutic effect or due to an AE of worsening of UC (or had a dose adjustment in the ITT analysis approach) were considered to be a treatment failure from the time of event onward.

TABLE 5
List of Analysis Approaches by Clinical Endpoint For Subjects Treated in
the Long-Term Extension

| | Analysis | Analysis |
|---|---|---|
| Clinical Endpoint | Population | Approach |
| Symptomatic remission over time through Week 92 | R | As observed, ITT, DATS |
| | NR | As observed, ITT |
| Partial Mayo remission over time through Week 92 | R | As observed, ITT, DATS |
| | NR | As observed, ITT |
| Symptomatic remission over time through Week 92 by | R | As observed, ITT |
| biologic- failure profile (biologic naive, biologic non-failure, | NR | ITT |
| biologic failure) | | |
| Partial Mayo remission over time through Week 92 by | R | As observed, ITT |
| biologic-failure profile (biologic naive, biologic non-failure, | NR | ITT |
| biologic failure) | | |
| Symptomatic remission over time through Week 92 among | R, NR | ITT |
| subjects who had achieved symptomatic remission at Week 44 | | |
| Symptomatic remission at both Week 44 and Week 92 among | R, NR | ITT |
| subjects who had achieved symptomatic remission at | | |
| maintenance baseline | | |
| Symptomatic remission at Week 92 among subjects who had | R, NR | ITT |
| achieved clinical remission at Week 44 | | |
| Symptomatic remission at both Week 44 and Week 92 among | R, NR | ITT |
| subjects who had achieved clinical remission at maintenance | | |
| baseline | | |
| Partial Mayo remission at Week 92 among subjects who had | R, NR | ITT |
| achieved partial Mayo remission at Week 44 | | |
| Partial Mayo remission at both Week 44 and Week 92 among | R, NR | ITT |
| subjects who had achieved partial Mayo remission at | | |
| maintenance baseline | | |
| Partial Mayo remission at Week 92 among subjects who had | R, NR | ITT |
| achieved clinical remission at Week 44 | | |
| Partial Mayo remission at both Week 44 and Week 92 among | R, NR | ITT |
| subjects who had achieved clinical remission at maintenance | | |
| baseline | | |
| The change from baseline (maintenance and induction) in partial | R, NR | As observed, ITT |
| Mayo score over time through Week 92 | | |
| Symptomatic remission/partial Mayo remission and not receiving | R, NR | ITT |
| corticosteroids at Week 92 | | |
| Symptomatic remission/partial Mayo remission and not receiving | R, NR | ITT |
| corticosteroids at Week 92 among subjects receiving | | |
| corticosteroids at maintenance baseline and among subjects | | |
| receiving corticosteroids at Week 44 | | |
| Mayo rectal bleeding subscore of 0 from Week 0 of induction | R, NR | ITT |
| study over time through Week 92 | | |
| Stool frequency subscore of 0 or 1 from Week 0 of induction | R, NR | ITT |
| study over time through Week 92 | | |
| Absolute stool number from induction baseline over time through | R | ITT |
| Week 92 | | |
| Not receiving concomitant corticosteroids at Week 92 among | R | ITT |
| subjects receiving concomitant corticosteroids at maintenance | | |
| baseline and among subjects receiving concomitant | | |
| corticosteroids at Week 44 | | |
| The change from maintenance baseline (or Week 44) in the | R | ITT |
| average daily prednisone-equivalent (P.Eq.) corticosteroid dose | | |
| (excluding budesonide and beclomethasone dipropionate) over time | | |
| through Week 92 among subjects receiving corticosteroids other | | |
| than budesonide and beclomethasone dipropionate at maintenance | | |
| baseline (or Week 44) | | |

Abbreviations: DATS = dose adjustment as a treatment strategy; ITT = intent to treat; LTE = long-term extension; NR = nonrandomized subjects at Week 0 of the maintenance study who were treated in the LTE; P.Eq .= prednisone equivalent; R = Randomized subjects at Week 0 of the maintenance study who were treated in the LTE

### Inflammatory Biomarkers

The following endpoints were summarized for both randomized and nonrandomized subjects at Week 0 of the maintenance study who were treated in the LTE based on the ITT analysis approach:
The change from baseline (maintenance and induction) in CRP, fecal lactoferrin, and fecal calprotectin concentrations over time through Week 92
Normalization of CRP, fecal lactoferrin, and fecal calprotectin over time through Week 92 among subjects with abnormal CRP, fecal lactoferrin, and fecal calprotectin, respectively, at induction baseline

### Health-Related Quality of Life

The following endpoints were summarized for randomized subjects at Week 0 of the maintenance study who were treated in the LTE based on the ITT analysis approach:
IBDQ
   The change from baseline (maintenance and induction) in the IBDQ score and each of the 4 IBDQ dimensions over time through Week 92
   A ≥16-point improvement from induction baseline in IBDQ over time through Week 92
   A ≥16-point improvement from induction baseline in IBDQ over time through Week 92 among subjects with a ≥16-point improvement in IBDQ (from induction baseline) at the maintenance baseline
   A ≥16-point improvement from induction baseline in IBDQ at both Week 44 and Week 92 among subjects with a ≥16-point improvement in IBDQ (from induction baseline) at maintenance baseline
   A ≥16-point improvement from induction baseline in IBDQ at both Weeks 68 and Week 92 among subjects with a ≥16-point improvement in IBDQ (from induction baseline) at Week 44
   IBDQ remission over time through Week 92
   IBDQ remission over time through Week 92 among subjects with IBDQ remission at the maintenance baseline
   IBDQ remission over time at both Week 44 and Week 92 among subjects with IBDQ remission at maintenance baseline
   IBDQ remission over time at both Week 68 and Week 92 among subjects with IBDQ remission at Week 44
SF-36
The change from baseline (maintenance and induction) in SF-36 physical component summary (PCS) and mental component summary (MCS) scores over time through Week 92
A ≥5-point improvement from induction baseline in SF-36 PCS and in SF-36 MCS over time through Week 92
A ≥5-point improvement from induction baseline in SF-36 PCS and in SF-36 MCS over time through Week 92 among subjects with a ≥5-point improvement in the SF-36 PCS and MCS, respectively, at the maintenance baseline
A ≥5-point improvement from induction baseline in SF-36 PCS and in SF-36 MCS at both Week 44 and Week 92 among subjects with a ≥5-point improvement in the SF-36 PCS and MCS, respectively, at maintenance baseline
A ≥5-point improvement from induction baseline in SF-36 PCS and in SF-36 MCS at both Week 68 and Week 92 among subjects with a 5-point improvement in the SF-36 PCS and MCS, respectively, at Week 44
   In addition, the following endpoints were summarized for nonrandomized subjects at Week 0 of the maintenance study who were treated in the LTE based on the ITT analysis approach:
The change from maintenance baseline in the IBDQ score over time through Week 92
A ≥16-point improvement from induction baseline in IBDQ over time through Week 92
IBDQ remission over time through Week 92
The change from maintenance baseline in SF-36 PCS and MCS over time through Week 92
A ≥5-point improvement from induction baseline in SF-36 PCS and in SF-36 MCS over time through Week 92

### Treated Subjects in the Long-Term Extension Who had a Dose Adjustment

For randomized subjects who had a dose adjustment prior to or at Week 76 and had data at least 16 weeks after dose adjustment, data at the time of dose adjustment and at the first visit ≥16 weeks after dose adjustment were summarized for the following endpoints:
Symptomatic remission
Partial Mayo remission
Partial Mayo score
CRP (mg/L)
Fecal calprotectin (mg/kg)
Fecal lactoferrin (μg/g)
   In addition, similar summaries were provided for symptomatic remission and partial Mayo remission by biologic-failure profile (biologic naïve, biologic nonfailure, and biologic failure). Subjects who were not in symptomatic remission/partial Mayo remission at the time of dose adjustment but were in symptomatic remission/partial Mayo remission at the first visit ≥16 weeks after dose adjustment were also summarized. In all of these analyses, subjects who had an ostomy or colectomy, or discontinued study agent due to lack of therapeutic effect or due to an AE of worsening of UC were considered to be a treatment failure from the time of event onward.

### Efficacy and Pharmacokinetics

The following efficacy endpoints were summarized by ustekinumab concentrations (<1^{st} quartile, ≥1st quartile and <2nd quartile, ≥2nd quartile and <3rd quartile, and ≥3rd quartile) at Week 92, and average trough serum ustekinumab concentration through Week 96 based on randomized subjects in maintenance who received ustekinumab in the LTE and did not have a dose adjustment:
Symptomatic remission at Week 92
Partial Mayo remission at Week 92
The change from maintenance baseline in CRP, fecal lactoferrin, and fecal calprotectin concentrations at Week 92
Normalization of CRP, fecal lactoferrin, and fecal calprotectin at Week 92 among subjects with abnormal CRP, fecal lactoferrin, and fecal calprotectin, respectively, at induction baseline

### Efficacy and Immunogenicity

The relationships between antibody to ustekinumab status through Week 96 and partial Mayo remission and symptomatic remission status at Week 92 were explored for randomized subjects in maintenance who were treated in LTE.

### Safety

For this CSR, safety summaries focused on all treated subjects who received at least 1 administration of study agent in the LTE. Summaries of safety were mainly based on data from Week 44 through Week 96, though some key safety analyses also included the data before Week 44. Additional summaries were also provided based on randomization status (i.e., randomized or nonrandomized in the maintenance study), up to the time of dose adjustment; and based on randomized subjects, including the data following dose adjustment.

### Adverse Events

Treatment-emergent AEs were coded in accordance with the Medical Dictionary for Regulatory Activities (MedDRA), version 21.1, using the lower-level term (LLT) as the description most closely related to the investigator's terminology, a preferred term (PT) describing a group of closely related LLTs, and the system-organ class (SOC), which is the broad category including related PTs.
The proportion of subjects with 1 or more of the following treatment-emergent AEs was summarized by treatment group:
Any AEs
SAEs
AEs leading to discontinuation of study agent
Injection-site reactions
Infections and serious infections
   An injection-site reaction is any adverse reaction at an SC study agent injection site and was recorded as an AE (and an injection-site reaction) by the investigator on the electronic case report form (eCRF).
   An infection was defined as any AE that was characterized by the investigator as an infection on the eCRF.
   Listings were provided for treatment-emergent SAEs, AEs leading to discontinuation of study agent, malignancies, serious major adverse cardiovascular events (MACE), embolic and thrombotic events and death.
   The number of events per hundred subject-years of follow-up and the number of subjects with events per hundred subject-years of follow-up were also summarized to adjust for potential differences in the duration of follow-up.
   In addition, the incidence of malignancies was to be described in this CSR.

### Laboratory Tests

The maximum postbaseline National Cancer Institute Common Terminology Criteria for Adverse Events (NCI-CTCAE) Toxicity Grade for laboratory values from Week 44 through Week 96 was summarized by laboratory test and by treatment group.
The laboratory values with maximum CTCAE grade ≥2 were also presented in listings. The NCI-CTCAE Toxicity grades are based on NCI-CTCAE version 4.03.

### Safety and Immunogenicity

The relationships between injection-site reactions from Week 44 through Week 96 and antibody to ustekinumab status through Week 96 were also explored for subjects who received ustekinumab SC during the LTE.

### Medical Resource Utilization and Health Economics

Summaries were provided for the following endpoints based on randomized subjects at Week 0 of the maintenance study who were treated in the LTE; no data-handling rules were applied:
The proportion of subjects with a UC disease-related hospitalization, or UC disease-related surgery, or both from induction Week 0 through Week 96
The proportion of subjects with a UC disease-related hospitalization, or UC disease-related surgery, or both from maintenance Week 0 through Week 96
The proportion of subjects with a UC disease-related hospitalization, or UC disease-related surgery, or both from Week 44 through Week 96
The change from baseline (maintenance and induction) in Productivity VAS over time through Week 92
The change from baseline (maintenance and induction) in each of the four impairment percentages from WPAI-GH over time through Week 92

### Subject and Treatment Information

### Subject Disposition and Study Completion/Withdrawal Information

### Distribution of Enrolled Subjects by Treatment Group and Region

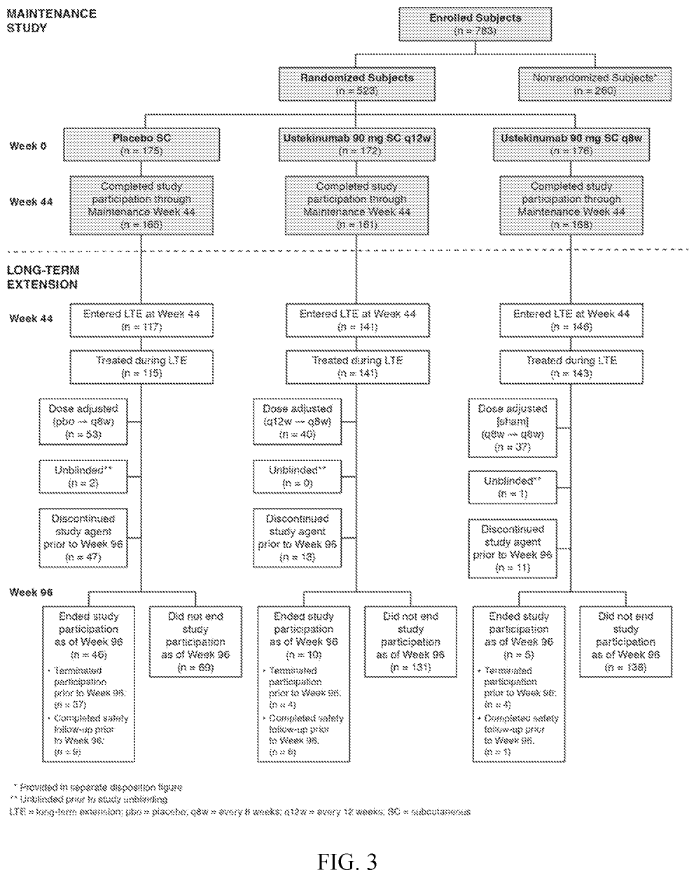

The disposition of subjects through Week 44 of this study was presented in the UC03001 44W CSR. A total of 588 subjects who completed the safety and efficacy evaluation at Week 44 and, in the opinion of the investigator, would benefit from continued treatment were treated in the LTE. Among these, 399 subjects were from the primary population for the maintenance study (i.e., were in clinical response to ustekinumab IV induction and were randomized at maintenance Week 0; FIGS. 3 and 189 subjects were not part of the primary population for the maintenance study (i.e., placebo induction responders and ustekinumab induction delayed responders [nonrandomized subjects]; FIG. 3).
The 399 subjects randomized at maintenance baseline who were treated during the LTE were as follows (FIG. 3):
Placebo SC: 115 subjects
Ustekinumab 90 mg SC q12w: 141 subjects
Ustekinumab 90 mg SC q8w: 143 subjects
   Subjects in the primary population of the maintenance study (i.e., those who were randomized at Week 0) who were treated in the LTE and whose UC disease activity worsened during the LTE were eligible for a single dose adjustment to ustekinumab 90 mg q8w (Section 0). A total of 32.6% (130 subjects) of the randomized population had a dose adjustment during the LTE.
   The 189 nonrandomized subjects in maintenance who were treated during the LTE were as follows (FIG. 3):
Placebo SC: 73 subjects who were in clinical response to placebo IV induction (responders to placebo IV induction) continued to receive placebo SC throughout maintenance and during the LTE until study unblinding, when they were discontinued from the study (see Section 0 for more details).
Ustekinumab 90 mg q8w: 116 subjects were ustekinumab induction delayed responders (ie, were not in clinical response to ustekinumab at induction Week 8 but were in clinical response at induction Week 16 after receiving a SC administration of ustekinumab at induction Week 8) and continued to receive ustekinumab 90 mg SC q8w throughout maintenance and into the LTE.
   Including all randomized and nonrandomized subjects, the 588 subjects were from 196 sites, 14.8% from Asia, 44.9% from Eastern Europe, and 40.3% from the Rest of World (including North America, Western Europe, Israel, Australia, and New Zealand).

### Study Participation Status Through Week 96

### Discontinuation of Study Agent

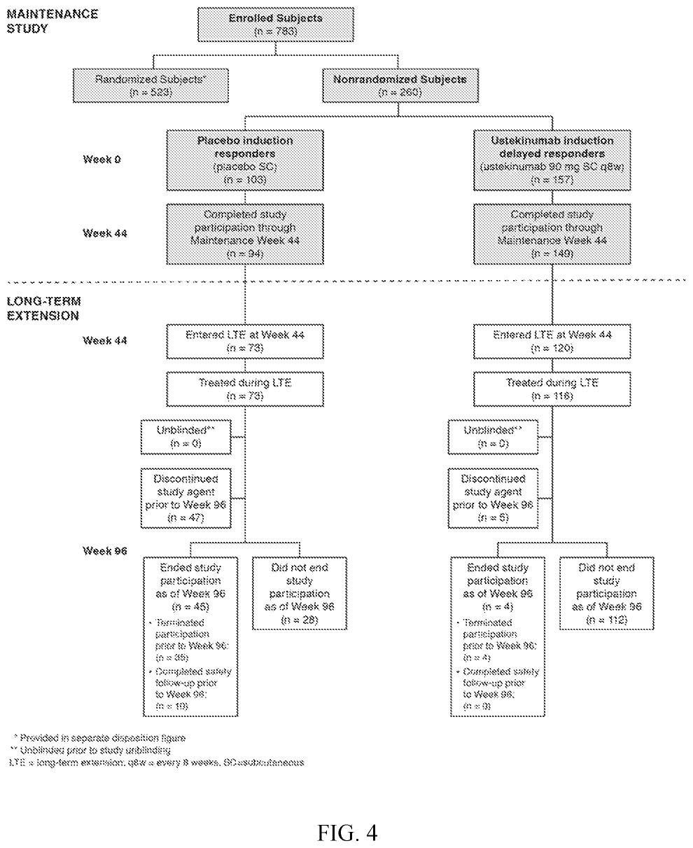

The numbers of subjects who were treated in the LTE and discontinued study agent prior to Week 96 are presented in FIG. 4. Subjects who discontinued study agent were to be followed for safety for approximately 20 weeks following their last dose of study agent.

### Randomized Subjects

The proportion of subjects from the randomized population who discontinued study agent prior to Week 96 was 17.8% (71 subjects; FIG. 4.
The proportions of subjects who discontinued study agent from each treatment group were 40.9% in the placebo group (including those subjects who were discontinued after study unblinding [29.6%]) and 8.5% in the combined ustekinumab group, with comparable proportions in the ustekinumab q12w and q8w groups (9.2% and 7.7%, respectively). The most common reasons for discontinuation of study agent in the combined ustekinumab group were Adverse event due to worsening of UC (2.5% [7 subjects]) and Other (2.5% [7 subjects]; most were reported as withdrawal of consent).

### Nonrandomized Subjects

The proportion of subjects from the nonrandomized population who discontinued study agent prior to Week 96 was 27.5% (52 subjects; FIG. 4).
The proportions of nonrandomized subjects who discontinued study agent from each treatment group were 64.4% (47 subjects) in the placebo induction responder group (including those subjects who were discontinued after study unblinding [39.7% (29 subjects)]) and 4.3% (5 subjects) in the ustekinumab induction delayed-responder group (Table 6). The most common reason for discontinuation of study agent among subjects in the ustekinumab induction delayed-responder group was Other (1.7% [2 subjects]; both were reported as withdrawal of consent).

TABLE 6
Number of Subjects Who Discontinued Study Agent Prior to Week 96 by Reason For Discontinuation;
Subjects Who Were Treated in the Long-Term Extension (CNTO1275UCO3001)

| | | | | | | Nonrandomized subjects | | |
|---|---|---|---|---|---|---|---|---|
| | | Randomized subjects ^{a} | | | | Responders to | Delayed | |
| | | Ustekinumab | | | | placebo IV | responders ^{d} | |
| | Placebo | 90 mg SC | 90 mg SC | | | induction | Ustekinumab | Overall |
| | SC ^{b} | q12w | q8w | Combined | Total | Placebo SC ^{c} | 90 mg SC q8w | total |
| Subjects who were | 115 | 141 | 143 | 284 | 399 | 73 | 116 | 588 |
| treated in the | | | | | | | | |
| long-term extension | | | | | | | | |
| Subjects who | 47 | 13 | 11 | 24 | 71 | 47 | 5 | 123 |
| discontinued | (40.9%) | (9.2%) | (7.7%) | (8.5%) | (17.8%) | (64.4%) | (4.3%) | (20.9%) |
| study agent | | | | | | | | |
| Reason for | | | | | | | | |
| discontinuation | | | | | | | | |
| Adverse event | 5 | 9 | 2 | 11 | 16 | 7 | 2 | 25 |
| | (4.3%) | (6.4%) | (1.4%) | (3.9%) | (4.0%) | (9.6%) | (1.7%) | (4.3%) |
| Worsening of UC | 5 | 6 | 1 | 7 | 12 | 7 | 1 | 20 |
| | (4.3%) | (4.3%) | (0.7%) | (2.5%) | (3.0%) | (9.6%) | (0.9%) | (3.4%) |
| Other than | 0 | 3 | 1 | 4 | 4 | 0 | 1 | 5 |
| worsening of UC | | (2.1%) | (0.7%) | (1.4%) | (1.0%) | | (0.9%) | (0.9%) |
| Lack of efficacy | 4 | 1 | 2 | 3 | 7 | 6 | 1 | 14 |
| | (3.5%) | (0.7%) | (1.4%) | (1.1%) | (1.8%) | (8.2%) | (0.9%) | (2.4%) |
| Did not show | 1 | 1 | 2 | 3 | 4 | 1 | 0 | 5 |
| improvement in UC | (0.9%) | (0.7%) | (1.4%) | (1.1%) | (1.0%) | (1.4%) | | (0.9%) |
| disease activity 16 | | | | | | | | |
| weeks following | | | | | | | | |
| dose adjustment | | | | | | | | |
| Lost to follow-up | 0 | 0 | 0 | 0 | 0 | 0 | 0 | 0 |
| Placebo subjects | 34 | 0 | 0 | 0 | 34 | 29 | 0 | 63 |
| discontinued after | (29.6%) | | | | (8.5%) | (39.7%) | | (10.7%) |
| study unblinding | | | | | | | | |
| Death | 0 | 0 | 0 | 0 | 0 | 0 | 0 | 0 |
| Other | 3 | 2 | 5 | 7 | 10 | 4 | 2 | 16 |
| | (2.6%) | (1.4%) | (3.5%) | (2.5%) | (2.5%) | (5.5%) | (1.7%) | (2.7%) |

Abbreviations: IV = intravenous; q8w = every 8 weeks; q12w = every 12 weeks; SC = subcutaneous; UC = ulcerative colitis
^{a}: Subjects who were in clinical response to ustekinumab IV induction dosing based on the treatment assignment by interactive web response system on entry into the maintenance study, regardless whether subjects had a dose adjustment during the long-term extension.
^{b}: Subjects who were in clinical response to ustekinumab IV induction dosing and were randomized to placebo SC on entry into the maintenance.
^{c}: Subjects who were in clinical response to placebo IV induction dosing and received placebo SC on entry into the maintenance study.
^{d}: Subjects who were not in clinical response to ustekinumab at induction Week 8 but were in clinical response at induction Week 16 after a SC administration of ustekinumab at induction Week 8.

### Termination of Study Participation

A summary of the subjects who entered the LTE and terminated study participation prior to Week 96 is presented below.

### Randomized Subjects

The proportion of randomized subjects who terminated study participation prior to Week 96 was 11.4% (46 subjects), including 32.5% (38 subjects) in the placebo group (with 25.6% [30 subjects] who were discontinued after study unblinding) and 2.8% (8 subjects) in the combined ustekinumab group. The most common reason for termination of study participation in the combined ustekinumab group was withdrawal of consent (2.4% [7 subjects]).

### Nonrandomized Subjects

The proportions of nonrandomized subjects who terminated study participation prior to Week 96 were 47.9% (35 subjects) in the placebo induction responder group (with 28.8% [21 subjects] who were discontinued after study unblinding) and 5.0% (6 subjects) in the ustekinumab induction delayed-responder group. The most common reason for termination of study participation in the ustekinumab induction delayed-responder group was withdrawal of consent (4.2% [5 subjects]).
The results of the subjects who were treated in the LTE and terminated study participation prior to Week 96 were similar to those presented above based on the subjects who entered the LTE.

### Study Agent Unblinding From Week 44 Through Week 96

Three subjects (all from the randomized population) who were treated in the LTE were unblinded prior to study unblinding from Week 44 through Week 96: 2 subjects in the placebo group and 1 subject in the ustekinumab q8w group. All 3 subjects were unblinded to treatment by the site to manage further medical treatment after discontinuation of study agent. All 3 subjects completed an early termination visit as required per protocol.

### Demographic and Baseline Characteristics

The demographic and baseline clinical disease characteristics were based on subjects who were treated in the LTE. The main analysis population is the population of randomized subjects; therefore, presentation of data focuses on these subjects. Data from the nonrandomized subjects are also provided with a focus on the ustekinumab induction delayed-responder group.

### Demographics

### Randomized Subjects

Among randomized subjects, 58.1% were male, 74.4% were white, the median age was 40.0 years, and the median weight was 71.60 kg.

### Nonrandomized Subjects

Generally similar demographic characteristics as those observed in the randomized population were observed among nonrandomized subjects; however, the ustekinumab induction delayed responders were more likely to be male compared with the randomized subjects.

### Clinical Disease Characteristics

The induction and maintenance baseline disease characteristics of the randomized subjects treated in the LTE were consistent with those of the overall randomized population in the maintenance study.

### Disease Characteristics at Maintenance Week 44

### Randomized Subjects

The clinical disease characteristics at Week 44 for randomized subjects who were treated in the LTE were generally similar for the ustekinumab q12w and q8w groups and numerically higher (e.g., Mayo score, CRP concentrations) or lower (e.g., subjects in remission) in the placebo group, indicating higher disease activity in the placebo group. Data for the ustekinumab q12w and q8w group, respectively, are presented below:
Proportion of subjects in clinical remission (global definition): 46.1% and 52.4%
Proportion of subjects with endoscopic healing: 56.7% and 61.5%
Mean Mayo score: 2.6 and 2.4
Median IBDQ score: 193.0 and 194.0
Median CRP concentration: 1.47 mg/L and 1.41 mg/L
Median fecal calprotectin concentration: 118.00 mg/kg and 158.00 mg/kg
Median fecal lactoferrin concentration: 9.08 μg/g and 13.30 μg/g
   The clinical disease characteristics at Week 44 for randomized subjects in the placebo group who were treated in the LTE were as follows:
Proportion of subjects in clinical remission (global definition): 34.8%
Proportion of subjects with endoscopic healing: 47.8%
Mean Mayo score: 3.2
Median IBDQ score: 185.0
Median CRP concentration: 2.56 mg/L
Median fecal calprotectin concentration: 368.00 mg/kg
Median fecal lactoferrin concentration: 28.95 μg/g

### Nonrandomized Subjects

The clinical disease characteristics at Week 44 among subjects in the ustekinumab induction delayed-responder group (received ustekinumab q8w during the LTE) compared with the clinical disease characteristics of randomized subjects from the ustekinumab q8w group were indicative of higher disease activity in the ustekinumab induction delayed-responder group (e.g., lower number of subjects in remission for the clinical efficacy endpoints, higher levels of inflammatory biomarkers); data presented below for each group, respectively:
Proportion of subjects in clinical remission (global definition): 38.8% and 52.4%
Proportion of subjects with endoscopic healing: 47.4% and 61.5%
Mean Mayo score: 3.2 and 2.4
Median IBDQ score: 189.5 and 194.0
Median CRP concentration: 1.72 mg/L and 1.41 mg/L
Median fecal calprotectin concentration: 324.00 mg/kg and 158.00 mg/kg
Median fecal lactoferrin concentration: 30.06 μg/g and 13.30 μg/g

### Prior and Concomitant Therapies

Concomitant UC medications and UC-related medication history presented are from Week 0 of the induction study for all subjects who were treated in the LTE.

### Concomitant Therapies

### Randomized Subjects

At induction baseline, 90.5% of randomized subjects treated in the LTE were receiving a concomitant UC medication. The overall proportions of subjects receiving corticosteroids, immunomodulatory drugs, and aminosalicylates were 50.1%, 29.3%, and 73.9%, respectively.

TABLE 7
Summary of UC-Related Concomitant Medications at Week 0 of the Induction Study;
Subjects Who Were Treated in the Long-Term Extension (CNTO1275UCO3001)

| | | | | | | Nonrandomized subjects | | |
|---|---|---|---|---|---|---|---|---|
| | | | | | | | Delayed | |
| | | Randomized subjects ^{a} | | | | Responders | responders ^{d} | |
| | | Ustekinumab | | | | to placebo | Ustekinumab | |
| | Placebo | 90 mg SC | 90 mg SC | | | IV induction | 90 mg SC | Overall |
| | SC ^{b} | q12w | q8w | Combined | Total | Placebo SC ^{c} | q8w | total |
| Subjects who were | 115 | 141 | 143 | 284 | 399 | 73 | 116 | 588 |
| treated in the | | | | | | | | |
| long-term extension | | | | | | | | |
| Any UC medication | 105 | 128 | 128 | 256 | 361 | 71 | 110 | 542 |
| | (91.3%) | (90.8%) | (89.5%) | (90.1%) | (90.5%) | (97.3%) | (94.8%) | (92.2%) |
| Corticosteroids use | 57 | 69 | 74 | 143 | 200 | 40 | 52 | 292 |
| | (49.6%) | (48.9%) | (51.7%) | (50.4%) | (50.1%) | (54.8%) | (44.8%) | (49.7%) |
| Corticosteroid | 46 | 58 | 65 | 123 | 169 | 34 | 41 | 244 |
| use (excl. | (40.0%) | (41.1%) | (45.5%) | (43.3%) | (42.4%) | (46.6%) | (35.3%) | (41.5%) |
| budesonide and | | | | | | | | |
| beclomethasone | | | | | | | | |
| dipropionate) | | | | | | | | |
| Budesonide | 12 | 11 | 9 | 20 | 32 | 7 | 10 | 49 |
| | (10.4%) | (7.8%) | (6.3%) | (7.0%) | (8.0%) | (9.6%) | (8.6%) | (8.3%) |
| Beclomethasone | 1 | 2 | 0 | 2 | 3 | 0 | 2 | 5 |
| dipropionate | (0.9%) | (1.4%) | | (0.7%) | (0.8%) | | (1.7%) | (0.9%) |
| subjects | | | | | | | | |
| Immunomodulatory | 39 | 37 | 41 | 78 | 117 | 25 | 43 | 185 |
| drugs | (33.9%) | (26.2%) | (28.7%) | (27.5%) | (29.3%) | (34.2%) | (37.1%) | (31.5%) |
| 6-mercaptopurine/ | 39 | 36 | 40 | 76 | 115 | 25 | 42 | 182 |
| azathioprine | (33.9%) | (25.5%) | (28.0%) | (26.8%) | (28.8%) | (34.2%) | (36.2%) | (31.0%) |
| Methotrexate | 0 | 1 | 1 | 2 | 2 | 0 | 1 | 3 |
| | | (0.7%) | (0.7%) | (0.7%) | (0.5%) | | (0.9%) | (0.5%) |
| Aminosalicylates | 86 | 115 | 94 | 209 | 295 | 56 | 91 | 442 |
| | (74.8%) | (81.6%) | (65.7%) | (73.6%) | (73.9%) | (76.7%) | (78.4%) | (75.2%) |

Abbreviations: IV = intravenous; q8w = every 8 weeks; q12w = every 12 weeks; SC = subcutaneous; UC = ulcerative colitis
^{a}: Subjects who were in clinical response to ustekinumab IV induction dosing based on the treatment assignment by interactive web response system on entry into the maintenance study, regardless whether subjects had a dose adjustment during the long-term extension.
^{b}: Subjects who were in clinical response to ustekinumab IV induction dosing and were randomized to placebo SC on entry into the maintenance.
^{c}: Subjects who were in clinical response to placebo IV induction dosing and received placebo SC on entry into the maintenance study.
^{d}: Subjects who were not in clinical response to ustekinumab at induction Week 8 but were in clinical response at induction Week 16 after a SC administration of ustekinumab at induction Week 8.

While the proportions of randomized subjects receiving corticosteroids and immunomodulatory drugs at induction baseline were balanced across the ustekinumab treatment groups, the proportions receiving aminosalicylates were 74.8%, 81.6%, and 65.7% in the placebo, ustekinumab q12w, and ustekinumab q8w groups, respectively.

### Nonrandomized Subjects

Concomitant UC medication use at induction baseline among the ustekinumab induction delayed-responder group was generally consistent with that of subjects in the randomized population from the ustekinumab q8w group.

### Medication History

### Randomized Subjects

The majority (93.7%) of subjects randomized in maintenance who were treated in the LTE demonstrated either an inadequate response to, or were intolerant of, corticosteroids and/or 6-mercaptopurine/azathioprine (6-MP/AZA), or demonstrated corticosteroid dependence at induction baseline. The history of response to and intolerance of UC medications was similar across all treatment groups. Among randomized subjects, 74.9% were refractory to, dependent on, or intolerant of corticosteroid treatment, and 54.9% were refractory to or intolerant of 6-MP/AZA treatment.

Of the subjects randomized in maintenance who were treated in the LTE, 55.9% had no documented history of biologic failure at induction baseline (53.1% were biologic-naïve and 2.8% were biologic-experienced but did not have documentation of biologic failure). Of the 44.1% of randomized subjects who had a documented history of biologic failure, the proportion of subjects was lower in the ustekinumab q12w group (37.6%) compared with the ustekinumab q8w group (49.7%).
Overall, subjects randomized in maintenance who were treated in the LTE had the following histories of biologic failure:
43.9% were biologic failures to at least 1 anti-TNF (regardless of vedolizumab).
   32.1% were biologic failures to only anti-TNF (not to vedolizumab).
12.0% were biologic failures to vedolizumab (regardless of anti-TNF).
11.8% were biologic failures to any anti-TNF and vedolizumab.

### Nonrandomized Subjects

The history of response to and intolerance of UC medications, and UC medication history among subjects in the ustekinumab induction delayed-responder group who were treated during the LTE was generally consistent with those of the randomized population from the ustekinumab q8w group.

### Randomized Subjects in Maintenance Who Had a Dose Adjustment

During the LTE, as early as Week 56, randomized subjects in maintenance whose UC disease activity was determined to have worsened based on the clinical judgment of the investigator were eligible for dose adjustment.

### Distribution of Subjects By Treatment Group

A total of 32.6% (130 subjects) of the randomized population had a dose adjustment as follows:
Among subjects randomized to placebo, 46.1% (53 subjects) had a dose adjustment to a ustekinumab 90 mg SC q8w dose regimen
Among subjects randomized to ustekinumab 90 mg SC q12w, 28.4% (40 subjects) had a dose adjustment to a ustekinumab 90 mg SC q8w regimen
Among subjects randomized to ustekinumab 90 mg SC q8w, 25.9% (37 subjects) had a sham dose adjustment (continued on the same dose regimen) The majority of subjects who underwent a dose adjustment did so prior to Week 68.

### Study Participation Status Through Week 96

### Discontinuation of Study Agent

Among the 130 subjects who had a dose adjustment during the LTE, 19 (14.6%) subjects discontinued study agent. The proportions of subjects who discontinued study agent were 15.1%, 17.5%, and 10.8% in the placebo→ustekinumab q8w, ustekinumab q12w→ustekinumab q8w, and ustekinumab q8w→ustekinumab q8w groups, respectively. The most common reason for discontinuation of study agent was Adverse event due to worsening of UC (5 [9.4%], 5 [12.5%], and 0 subjects in the placebo→ustekinumab q8w, ustekinumab q12w→ustekinumab q8w, and ustekinumab q8w→ustekinumab q8w groups, respectively).

### Termination of Study Participation

Among the 130 subjects who had a dose adjustment during the LTE, 116 (89.2%) subjects did not end study participation as of Week 96. Prior to Week 96, a total of 5 (3.8%) subjects terminated study participation (3 [5.7%], 1 [2.5%], and 1 [2.7%] subjects in the placebo→ustekinumab q8w, ustekinumab q12w→ustekinumab q8w, and ustekinumab q8w→ustekinumab q8w groups, respectively).

### Demographic and Baseline Characteristics

The baseline demographics and disease characteristics of randomized subjects who had a dose adjustment during the LTE were generally consistent with those of the randomized population.

### Demographics

The demographic characteristics at induction baseline for randomized subjects who had a dose adjustment during the LTE were generally well balanced across treatment groups. Overall, 62.3% were male, 75.4% were white, the median age was 40.0 years, and the median weight was 73.60 kg.

### Clinical Disease Characteristics

### Disease Characteristics at Maintenance Week 44

The clinical disease characteristics at Week 44 for randomized subjects who were treated and had a dose adjustment in the LTE were generally similar for the ustekinumab q12w→ustekinumab q8w and ustekinumab q8w→ustekinumab q8w groups (as presented below):
Proportion of subjects in clinical remission (global definition): 40.0% and 51.4%
Proportion of subjects with endoscopic healing: 55.0% and 54.1%
Mean Mayo score: 2.9 and 2.8
Median IBDQ score: 184.0 and 180.0
Median CRP concentration: 1.67 mg/L and 3.15 mg/L
Median fecal calprotectin concentration: 152.0 mg/kg and 284.00 mg/kg
Median fecal lactoferrin concentration: 15.36 μg/g and 24.22 μg/g
   The clinical disease characteristics at Week 44 for randomized subjects in the placebo group who had a dose adjustment to ustekinumab q8w during the LTE were as follows:
Proportion of subjects in clinical remission (global definition): 26.4%
Proportion of subjects with endoscopic healing: 37.7%
Mean Mayo score: 4.1
Median IBDQ score: 178.0
Median CRP concentration: 2.67 mg/L
Median fecal calprotectin concentration: 726.50 mg/kg
Median fecal lactoferrin concentration: 52.91 μg/g

### Prior and Concomitant Therapies

### Concomitant Medications

At induction baseline, 89.2% of subjects in maintenance who had a dose adjustment in the LTE were receiving a concomitant UC medication (88.7%, 85.0%, and 94.6% of subjects in the placebo→ustekinumab q8w, ustekinumab q12w→ustekinumab q8w, and ustekinumab q8w→ustekinumab q8w groups, respectively). The overall proportions of subjects receiving corticosteroids, immunomodulatory drugs, and aminosalicylates were 55.4%, 22.3%, and 70.0%, respectively. The proportions of subjects receiving each type of UC medication in the placebo→ustekinumab q8w, ustekinumab q12w→ustekinumab q8w, and ustekinumab q8w→ustekinumab q8w groups, respectively, were as follows:
Corticosteroids: 49.1%, 60.0%, and 59.5%
Immunomodulatory drugs: 26.4%, 17.5%, and 21.6%
Aminosalicylates: 73.6%, 70.0%, and 64.9%

### Medication History

The majority (95.4%) of subjects randomized in maintenance who had a dose adjustment in the LTE demonstrated either an inadequate response to, or were intolerant of, corticosteroids and/or 6-MP/AZA, or demonstrated corticosteroid dependence at induction baseline. Overall, 80.8% were refractory to, dependent on, or intolerant of corticosteroid treatment, and 58.5% were refractory to or intolerant of 6-MP/AZA treatment.
Of the subjects randomized in maintenance who had a dose adjustment in the LTE, 41.5% had no documented history of biologic failure at induction baseline (all were biologic-naïve). Of the 58.5% of subjects who had a documented history of biologic failure, the proportions of subjects across dose adjustment groups were comparable (56.6%, 60.0%, and 59.5% in the placebo→ustekinumab q8w, ustekinumab q12w→ustekinumab q8w, and ustekinumab q8w→ustekinumab q8w groups, respectively).
Subjects randomized in maintenance who had a dose adjustment in the LTE were more likely to have had a history of biologic failure than the overall randomized population treated in the LTE.
The proportions of dose-adjusters with a history of biologic failure in the following categories were as follows:
57.7% were biologic failures to at least 1 anti-TNF (regardless of vedolizumab)
   39.2% were biologic failures to only anti-TNF (not to vedolizumab)
19.2% were biologic failures to vedolizumab (regardless of anti-TNF)
18.5% were biologic failures to any anti-TNF and vedolizumab

### Protocol Deviations

From Week 44 through Week 96, 27 subjects (4.6%) had the following major protocol deviations:
2 subjects (0.3%) were reported to have met withdrawal criteria but were not withdrawn.
15 subjects (2.6%) were reported to have received the wrong treatment or incorrect dose.
11 subjects (1.9%) were reported to have had protocol deviations for reasons not listed above (eg, “other”).
   Subjects may have been counted in more than 1 category or may have had more than 1 deviation within a category.

### Study Agent Administration Deviations

Among subjects who were treated in the LTE, there were 15 reported study agent administration deviations; all 15 were classified as major protocol deviations, as noted above.

### Randomized Subjects

A total of 11 subjects (5 from the placebo group and 6 from the ustekinumab q12w group) had their dose erroneously adjusted to ustekinumab q8w at the Week 56 visit. Of the 5 subjects from the placebo group, 3 subjects continued on the adjusted dose (ustekinumab q8w) and 2 subjects were adjusted back to placebo. All subjects from the ustekinumab q12w group who were erroneously adjusted were returned to q12w dosing at the subsequent visit.
A total of 2 subjects (both from the ustekinumab q12w group) were not administered the assigned syringe during a visit and were, instead, administered an incorrect syringe (1 subject incorrectly received placebo during a visit, and 1 subject incorrectly received ustekinumab instead of placebo during a visit). The subjects were returned to their assigned dosing at the subsequent visit.

### Nonrandomized Subjects

Two subjects were not administered the assigned syringe during a visit and were, instead, administered an incorrect syringe; the subjects were returned to their assigned dosing at the subsequent visit. One subject was from the placebo induction responder group and was administered expired study agent at the Week 60 visit; the subject incorrectly received ustekinumab instead of placebo; the ustekinumab that was administered was also expired. The subject was followed for safety events and no AEs were reported. One subject was from the ustekinumab induction delayed-responder group and incorrectly received placebo at the Week 56 visit instead of ustekinumab.

### Met Withdrawal Criteria but Not Withdrawn

Among subjects who were treated in the LTE, there were 2 subjects (both from the nonrandomized placebo induction responder group) identified as having met withdrawal criteria but were not withdrawn. Both subjects were identified with worsening UC with no improvement after 16 weeks; however, both deviations were incorrectly reported as one subject discontinued treatment with study agent after reporting an AE of worsening UC within the 16-week period specified per protocol and the other subject did not report an AE of worsening UC during the LTE.

### Disallowed Concomitant Medication Deviations

Administration of concomitant therapy was at the discretion of the investigator with the exception of medications expressly prohibited by the protocol. No subject initiated a prohibited medication during the LTE.

### Other Major Protocol Deviations

Among subjects who were treated in the LTE, there were 11 subjects who were reported to have protocol deviations classified as “other.”
Among randomized subjects, 10 subjects were reported with “other” protocol deviations including 2 subjects in the placebo group and 4 subjects each in the ustekinumab q12w and q8w groups.
Among subjects in the placebo group:
   1 subject underwent local testing for serum ustekinumab levels and remained in the study.
   1 subject did not have clinical laboratory assessments performed at Week 56 and Week 68, so results were not available prior to next dosing visit; chemistry and hematology laboratory values were within normal ranges specified by the central lab on subsequent testing.
Among subjects in the ustekinumab q12w group:
   4 subjects had study agent administered that was later declared unfit for use; all subjects were monitored for safety events following administration, but no adverse safety events were identified.
Among subjects in the ustekinumab q8w group:
   3 subjects were not administered a urine pregnancy test at a dosing visit; testing resumed at the subsequent visit with no subjects reporting pregnancy.
   1 subject was not administered a TB assessment at the Week 60 visit; a TB evaluation was completed at the subsequent visit with no signs of active TB.
      Among nonrandomized subjects, a protocol deviation classified as “other” was reported in 1 subject from the placebo induction responder group who was administered expired study agent at the Week 60 visit, as previously described.

### Summary and Impact of Protocol Deviations

Overall, among the 399 randomized subjects and 189 nonrandomized subjects treated in the LTE, protocol deviations were reported in 23 (5.8%) subjects and 4 (2.1%) subjects, respectively. Most deviations (15 of 27 subjects) were classified as “Received the wrong treatment or incorrect dose”. Subjects on ustekinumab who were identified to have received an incorrect dose were analyzed in their assigned treatment groups, including subjects who had an error in dose adjustment; subjects on placebo who were identified to have received ustekinumab were considered to have a dose adjustment and were analyzed in the ustekinumab q8w group for safety from the time the subject received ustekinumab. Deviations related to protocol-specified procedures (i.e., study drug monitoring, urine pregnancy testing, and TB risk assessment) were addressed with the site and reviewed for impact on patient safety; no safety issues were identified.
During the study, specific deviations were addressed at the site level as well as through study-wide site communications and trainings. Issues were addressed during the conduct of the study with appropriate corrective and preventative action before DBL.
In summary, protocol deviations varied in nature and were determined not to have clinically relevant impact on data integrity or subject safety. Similarly, there was no notable effect of deviations on the safety profile of ustekinumab in this study; overall, the safety profile observed was consistent with the disease under study and the labeled safety information for ustekinumab in other indications.

### Treatment Compliance

Doses of study agent were administered by appropriately licensed and authorized health professionals according to the treatment groups assigned by the IWRS. Compliance with the treatment assignments was controlled by the study site personnel. Site personnel administered the study agent and recorded the amount of study agent given. A site monitor designated by the sponsor monitored all subject eCRFs. During these monitoring visits, all procedures were evaluated for compliance with the protocol. Missed study visits were recorded on the eCRF. Site monitors designated by the sponsor verified source documents, performed study agent accountability, and ensured overall site compliance. Subject charts were reviewed and compared with data entries on the eCRFs to ensure consistency. Study agent was not to be used for any purpose other than that outlined in the protocol. Used vials and syringes of study agent were retained at the site until the study agent accountability forms were checked by the site monitor.

### Extent of Exposure

In total, 454 subjects received at least 1 dose of ustekinumab during the LTE.
Placebo: 188 subjects received placebo (ustekinumab dose of 0.0 mg)
90 mg q12w: 141 subjects received a median cumulative dose of 450.0 mg
90 mg q8w: 353 subjects received a median cumulative dose of 630.0 mg
   Subjects who were randomized in maintenance and were treated in the LTE received study agent from Week 44 through Week 96 or up to the time of dose adjustment as follows:
Placebo: 115 subjects received placebo (ustekinumab dose of 0.0 mg)
90 mg q12w: 141 subjects received a median cumulative dose of 450.0 mg
90 mg q8w: 143 subjects received a median cumulative dose of 630.0 mg
   A total of 116 subjects in the ustekinumab induction delayed-responder group who were treated in the LTE (receiving ustekinumab 90 mg SC q8w) received a median cumulative dose of 630.0 mg from Week 44 through Week 96.
   A total of 73 subjects in the placebo induction responder group continued to receive placebo in the LTE. One subject in this group received ustekinumab. Safety data for this subject prior to the receipt of ustekinumab was included in the placebo group and safety data from the time the subject received ustekinumab was included in the ustekinumab q8w group as appropriate.

### Extent of Exposure Among Subjects Who Had a Dose Adjustment

Subjects who were randomized to ustekinumab in maintenance and had a dose adjustment during the LTE received ustekinumab from the time of dose adjustment onward through Week 96 as follows:
placebo→ustekinumab q8w: 53 subjects received a median cumulative dose of 450.0 mg
ustekinumab q12w→ustekinumab q8w: 40 subjects received a median cumulative dose of 270.0 mg
ustekinumab q8w→ustekinumab q8w: 37 subjects received a median cumulative dose of 180.0 mg

### Pharmacokinetics and Immunogenicity Results

### Pharmacokinetics

All treated subjects who received at least 1 administration of ustekinumab during the LTE were included in the PK analyses. For randomized subjects, serum ustekinumab concentrations are summarized up to the time of dose adjustment. In addition, summaries of ustekinumab concentration after the time of dose adjustment are provided for randomized subjects who had a dose adjustment. For nonrandomized subjects, ustekinumab concentration data was summarized for ustekinumab induction delayed responders. Results from Week 44 through Week 92 are summarized in this report while results from Week 0 through Week 44 were presented in the UCO3001 44W.
A total of 337 subjects who were randomized into maintenance continued into the LTE and received ustekinumab, including 141 subjects who received ustekinumab 90 mg SC q12w and 143 subjects who received ustekinumab 90 mg SC q8w, and 53 placebo subjects who had a dose adjustment during the LTE to ustekinumab q8w. Of the 141 randomized subjects receiving ustekinumab q12w in the LTE, 40 subjects had a dose adjustment to ustekinumab q8w. Of the 143 randomized subjects receiving ustekinumab q8w in the LTE, 37 subjects underwent a sham dose adjustment (i.e., continued to receive ustekinumab q8w).

### Randomized Subjects

Based on the study visit schedule, blood samples for the measurement of serum ustekinumab concentrations were collected every 24 weeks from Week 44 (i.e., at Week 44, Week 68, and Week 92). Accordingly, concentration data for subjects receiving ustekinumab q12w up to the time of dose adjustment were available 8 weeks after the respective ustekinumab dose administration at Week 36, Week 60, and Week 84, but not at trough. On the other hand, concentration data were available for subjects receiving ustekinumab q8w 4 weeks after the respective ustekinumab dose administration at Week 40, Week 64, and Week 88, but not at trough.
Randomized subjects who received ustekinumab during the LTE had sustained levels of ustekinumab throughout the LTE. At the start of the LTE at Week 44 (which corresponds to 8 weeks after the last maintenance dose in the ustekinumab q12w group, and 4 weeks after the last maintenance dose in the ustekinumab q8w group), median [mean] serum ustekinumab was approximately 3-fold greater in the ustekinumab q8w group (9.41 [8.84] g/mL) than in the q12w group (2.50 [3.02] g/mL). Subjects randomized to ustekinumab q12w in maintenance who continued to receive 90 mg ustekinumab in the LTE (i.e., at Week 48, Week 60, Week 72, and Week 84) had median ustekinumab concentrations ranging from 2.13 μg/mL to 2.59 μg/mL, 8 weeks after ustekinumab dosing, over the time period from Week 68 to Week 92. Subjects randomized to ustekinumab q8w in maintenance who continued to receive 90 mg ustekinumab in the LTE (i.e., at Week 48, Week 56, Week 64, Week 72, Week 80, and Week 88) had median ustekinumab concentrations ranging from 6.38 μg/mL to 6.65 μg/mL, 4 weeks after ustekinumab dosing, over the time period from Week 68 to Week 92.

TABLE 8
Summary of Serum Ustekinumab Concentrations (microgram/mL)
at Week 44, Week 68, and Week 92; Randomized Subjects in
Maintenance Who Received Ustekinumab in the Long-Term
Extension (CNTO1275UCO3001)

| | 90 mg SC q12w ^{a} | 90 mg SC q8w ^{a} |
|---|---|---|
| Randomized subjects in | 141 | 143 |
| maintenance who received | | |
| ustekinumab in the | | |
| long-term extension | | |
| Week 44 | | |
| N | 127 | 134 |
| Mean (SD) | 3.02 (2.070) | 8.84 (3.561) |
| Median | 2.50 | 9.41 |
| IQ range | (1.74; 3.82) | (5.92; 11.33) |
| Range | (0.0; 10.3) | (1.8; 20.1) |
| Week 68 | | |
| N | 113 | 109 |
| Mean (SD) | 2.98 (2.072) | 6.72 (2.743) |
| Median | 2.59 | 6.38 |
| IQ range | (1.81; 3.62) | (5.33; 8.54) |
| Range | (0.0; 14.5) | (0.3; 14.7) |
| Week 92 | | |
| N | 59 | 54 |
| Mean (SD) | 2.55 (1.736) | 6.61 (2.481) |
| Median | 2.13 | 6.65 |
| IQ range | (1.49; 2.74) | (5.22; 8.53) |
| Range | (0.2; 8.4) | (0.6; 13.4) |

Abbreviations: IQ = interquartile; q8w = every 8 weeks; q12w = every 12 weeks; SC = subcutaneous; SD = standard deviation
^{a}: Includes data from Week 44 through Week 92, or up to the time of dose adjustment for subjects who had a dose adjustment to ustekinumab 90 mg SC q8w (or a sham dose adjustment for the ustekinumab 90 mg SC q8w group) during the long-term extension.

These results indicate that randomized subjects who continued receiving either the ustekinumab q12w or q8w dose regimen in the LTE had sustained and consistent levels of ustekinumab through Week 92 of the LTE that were generally comparable with serum ustekinumab levels observed during the maintenance phase of the study.

### Randomized Subjects Who Had a Dose Adjustment

Randomized subjects in the maintenance study whose UC disease activity worsened during the LTE were eligible, beginning at Week 56, for a single dose adjustment. There were 3 possible scenarios for those who met the criteria for dose adjustment:
Subjects randomized to placebo had a dose adjustment to ustekinumab 90 mg q8w
Subjects randomized to ustekinumab 90 mg q12w had a dose adjustment to ustekinumab 90 mg q8w
Subjects randomized to ustekinumab 90 mg q8w continued receiving the same dose regimen (sham dose adjustment)
   Because the subjects who had a dose adjustment initiated ustekinumab q8w at different visits, concentration data summaries for these subjects are not representative of the expected concentrations over time for those on ustekinumab q8w. Nevertheless, as expected, serum ustekinumab concentrations increased following dose adjustment from placebo to ustekinumab q8w. Specifically, the median serum ustekinumab concentration increased from 0.0 μg/mL at Week 44 to 4.70 μg/mL and 3.64 μg/mL at Week 68 and Week 92, respectively.
   In subjects randomized to ustekinumab q12w, serum ustekinumab concentrations at Week 44 were similar between subjects who underwent a dose adjustment compared with those who did not (2.51 μg/mL and 2.50 μg/mL, respectively). After dose adjustment from ustekinumab q12w to ustekinumab q8w, median serum ustekinumab concentrations were 2.97 μg/mL and 3.83 μg/mL at Week 68 and Week 92, respectively.
   Among subjects randomized to the ustekinumab q8w group, median ustekinumab concentrations at Week 44 through Week 92 were generally comparable between subjects who underwent a dose adjustment compared with those who did not need dose adjustment.

### Nonrandomized Subjects (Ustekinumab Induction Delayed Responders)

Delayed responders to ustekinumab induction were subjects who did not respond to the Week 0 ustekinumab IV induction dose, received ustekinumab 90 mg SC at induction Week 8, and were in clinical response at induction Week 16. These subjects continued to receive SC ustekinumab 90 mg q8w in the maintenance study and the LTE.
At Week 44, the median serum ustekinumab concentration among subjects in the ustekinumab induction delayed-responder group (7.83 μg/mL) was slightly lower than that of subjects who responded to a single ustekinumab IV induction dose and were randomized to ustekinumab q8w and did not have a dose adjustment (9.67 μg/mL). This difference in ustekinumab concentration was no longer apparent at Week 68 and Week 92 where the median concentrations in delayed responders (6.21 μg/mL and 5.94 μg/mL, respectively) were comparable to those in subjects randomized to ustekinumab q8w and did not have a dose adjustment (6.49 μg/mL and 6.66 μg/mL, respectively).

### Immunogenicity

Immunogenicity (antibodies to ustekinumab) analyses were conducted for all treated subjects who received ustekinumab and for randomized subjects. The relationship between antibodies to ustekinumab and serum ustekinumab concentration in randomized subjects is also discussed.
The incidence of antibodies to ustekinumab was low through Week 96 of the LTE following treatment with ustekinumab.

### Immunogenicity Through Week 96

### Subjects Who Received Ustekinumab in Induction, Maintenance, and the Long-Term Extension

Among 400 subjects who received ustekinumab in maintenance and continued on ustekinumab in the LTE (this population comprised subjects who achieved clinical response after ustekinumab IV induction and were randomized to ustekinumab SC in maintenance, and those who were delayed responders at induction Week 16 and received SC maintenance therapy thereafter), 22 (5.5%) were positive for antibodies to ustekinumab between induction Week 0 and Week 96 of the LTE. The incidence of antibodies to ustekinumab in this group of subjects is considered the most relevant given that this is reflective of the manner in which ustekinumab is used in clinical practice. Most of the subjects (18 of 22 subjects) who were positive for antibodies to ustekinumab had titers at or below 1:800; 4 of the 22 subjects (18.2%) were positive for neutralizing antibodies (NAbs).

### Randomized Subjects

A total of 399 randomized subjects in maintenance (284 to ustekinumab and 115 to placebo) were treated during the LTE and had appropriate samples at some time through Week 96 to assess their antibody status to ustekinumab. The overall incidence of antibodies to ustekinumab among randomized subjects was 6.8% (27 of 399 subjects. Among subjects who did not have a dose adjustment (including sham adjustment) in the LTE, the incidence of antibodies to ustekinumab was similar in subjects who received ustekinumab q12w (5.0%) compared with subjects who received ustekinumab q8w (4.7%), but higher among subjects who continued to receive placebo after ustekinumab induction and did not have a dose adjustment (8.1%). The incidence of antibodies to ustekinumab was also higher in subjects who had a dose adjustment to ustekinumab q8w from placebo (13.2%) or from ustekinumab q12w (7.5%). Accordingly, the incidence of antibodies was higher among subjects who were receiving intermittent ustekinumab therapy (ie, subjects who received ustekinumab during induction and were randomized to placebo in maintenance, or subjects who received ustekinumab during induction, were randomized to placebo in maintenance, and had a dose adjustment to ustekinumab during the LTE) compared to those who were on continuous ustekinumab therapy.
Most of the randomized subjects (23 of 27 subjects) who were positive for antibodies to ustekinumab had titers at or below 1:800. Of the 27 randomized subjects who were positive for antibodies to ustekinumab through Week 96 of the LTE, 8 (29.6%) subjects were positive for NAbs.

TABLE 9
Summary of Antibody to Ustekinumab Status Through Week 96; Subjects Who
Continued on Ustekinumab in the Long-Term Extension (CNTO1275UCO3001)

| | | | | | Nonrandomized | |
|---|---|---|---|---|---|---|
| | Randomized subjects | | | | subjects | |
| | | | 90 mg SC | 90 mg SC | Delayed | |
| | 90 | 90 | q12w → 90 | q8w → 90 | responders | |
| | mg SC | mg SC | mg SC | mg SC | received 90 | |
| | q12w ^{a} | q8w ^{a} | q8w ^{b} | q8w ^{b} | mg SC q8w ^{c} | Total |
| Subjects who were treated | 101 | 106 | 40 | 37 | 116 | 400 |
| in the long-term extension | | | | | | |
| Through Week 96 | | | | | | |
| Subjects with appropriate samples ^{d} | 101 | 106 | 40 | 37 | 116 | 400 |
| Subjects positive for antibodies | 5 | 5 | 3 | 2 | 7 | 22 |
| to ustekinumab at any time ^{e,f} | (5.0%) | (4.7%) | (7.5%) | (5.4%) | (6.0%) | (5.5%) |
| Titers | | | | | | |
| 1:50 | 1 | 0 | 0 | 0 | 0 | 1 |
| 1:100 | 1 | 0 | 0 | 0 | 3 | 4 |
| 1:200 | 1 | 2 | 0 | 1 | 0 | 4 |
| 1:400 | 2 | 1 | 2 | 0 | 2 | 7 |
| 1:800 | 0 | 1 | 0 | 0 | 1 | 2 |
| 1:1600 | 0 | 1 | 0 | 1 | 1 | 3 |
| 1:12800 | 0 | 0 | 1 | 0 | 0 | 1 |
| Subjects negative for antibodies | 96 | 101 | 37 | 35 | 109 | 378 |
| to ustekinumab ^{f,g} | (95.0%) | (95.3%) | (92.5%) | (94.6%) | (94.0%) | (94.5%) |

Abbreviations: IV = intravenous; q8w = every 8 weeks; q12w = every 12 weeks; SC = subcutaneous
^{a}: Subjects who were in clinical response to ustekinumab IV induction dosing and were randomized the specified treatment on entry into the maintenance, and did not have a dose adjustment during the long-term extension.
^{b}: Subjects who had a dose adjustment to ustekinumab 90 mg SC q8w or a sham dose adjustment during the long-term extension.
^{c}: Subjects who were not in clinical response to ustekinumab at induction Week 8 but were in clinical response at induction Week 16 after a SC administration of ustekinumab at induction Week 8, initiate ustekinumab 90 mg SC q8w on entry into the maintenance.
^{d}: Subjects who had 1 or more samples obtained after their first study agent administration of the induction study through the evaluation visit.
^{e}: Subjects who had at least 1 positive sample at any time after their first study agent administration of the induction study through the evaluation visit.
^{f}: Denominator is subjects with appropriate samples.
^{g}: Excludes subjects who were positive for antibodies at any time.

### All Treated Subjects

A total of 515 all-treated subjects (62 received placebo in maintenance and LTE; 453 received ustekinumab in maintenance or LTE) who received at least 1 dose of ustekinumab during induction or maintenance through Week 96 of LTE had appropriate samples for antibodies to ustekinumab. Of the 515 subjects, 34 (6.6%) were positive for antibodies to ustekinumab through Week 96 of this study. Most of the subjects (29 of 34 subjects) who were positive for antibodies to ustekinumab had titers at or below 1:800.
Of the 34 all-treated subjects who were positive for antibodies to ustekinumab through Week 96 of the LTE, 8 subjects (23.5%) were positive for NAbs.

### Immunogenicity and Pharmacokinetics

The relationship between serum ustekinumab concentrations and antibody to ustekinumab status (positive or negative) through Week 96 of the LTE was evaluated among subjects randomized to ustekinumab.
In each ustekinumab SC treatment group (q12w and q8w), median serum ustekinumab concentrations were above the limit of quantification but lower over time in subjects who were positive for antibodies to ustekinumab compared with levels in subjects who were negative for antibodies to ustekinumab. Caution should be exercised in interpreting these data due to the small number of subjects who were positive for antibodies to ustekinumab.

### Pharmacology Summary

Following continued treatment with ustekinumab 90 mg SC q8w or q12w during the LTE, sustained levels of ustekinumab were observed through Week 92 that were generally consistent with serum ustekinumab levels observed for these treatment groups during the maintenance study.
The incidence of antibodies to ustekinumab was low through Week 96 of the LTE.
   Among 400 subjects who received ustekinumab during induction, maintenance, and the LTE, 22 subjects (5.5%) were positive for antibodies to ustekinumab through Week 96 with most of the subjects having antibody titers ≤1:800.
   Among 515 all-treated subjects who received at least 1 dose of ustekinumab during induction or maintenance through Week 96 of LTE, 34 subjects (6.6%) were positive for antibodies to ustekinumab through Week 96 of this study with most of the subjects having antibody titers 1:800.
      The incidence of antibodies to ustekinumab appeared higher in subjects randomized to placebo in this maintenance study (who originally received 1 infusion of ustekinumab during induction), or those who needed dose adjustment from placebo or ustekinumab q12w during the LTE.
      Of the 34 all-treated subjects who were positive for antibodies to ustekinumab, 8 (23.5%) subjects were positive for NAbs.

### Efficacy Results

### Populations for Analysis

The analysis population that is the focus for this CSR consists of the randomized subjects who were treated in the LTE. Additionally, selected summaries were provided for the randomized subjects who had a dose adjustment during the LTE and for nonrandomized subjects who were treated in the LTE with a focus on subjects in the ustekinumab induction delayed-responder group.
Further, selected summaries are provided for all subjects who were randomized at maintenance baseline (i.e., regardless of whether they were treated in the LTE); similar summaries are provided for all subjects who were not randomized at maintenance baseline.

### Efficacy Analyses

The intent of the efficacy analyses in the LTE was to assess maintenance of clinical benefit from the end of the main study (Week 44) through Week 92, though the data before Week 44 were also included.
It is important to note that subjects entered the LTE based on investigator determination as to whether the subject would benefit from continuation of treatment. Furthermore, the placebo group represents a subpopulation of UC patients who either were long-term responders to ustekinumab induction therapy (i.e., were re-randomized to placebo maintenance) or placebo induction responders with a longer latency of disease. For these reasons, and because placebo subjects were to terminate from study participation after study unblinding, a direct comparison of findings between treatment groups was not warranted, and no statistical comparisons were performed. The primary focus of this CSR is on the subjects treated with ustekinumab 90 mg SC q12w and 90 mg SC q8w.
Different analysis approaches were adopted. In the as-observed analysis approach, at each analysis time point, only those subjects who had data available or who had a treatment failure prior to that time point (considered as nonresponders) were included in the analysis. This approach was considered reasonable as only those patients with missing data not related to treatment failure (presumably missing at random) were excluded from the analysis.
In the ITT analysis approach, the number of subjects included in the analysis was fixed over time. As it was expected more subjects underwent dose adjustment (a treatment failure criterion) or discontinued study agent (whether or not due to lack of therapeutic effect or due to an AE of worsening of UC) over time, the proportion of subjects who achieved binary endpoints was expected to decrease over time. As such, the ITT analysis approach was considered conservative.
The conservative ITT analysis approach was used as the default for efficacy analyses. However, analyses based on the as-observed analysis approach were performed for key efficacy endpoints such as symptomatic remission, partial Mayo remission and the change from baseline in partial Mayo score, and were considered to be more reasonably reflective of efficacy in the LTE. The dose-adjustment-as-a-treatment-strategy analysis approach was also included and considered pragmatic as it reflects the clinical practice where treatments are optimized either through increases in dose or dosing frequency.

### Randomized Subjects in Maintenance Who Were Treated in the Long-Term Extension

### Clinical Efficacy

### Symptomatic Remission

Symptomatic Remission from Week 44 Through Week 92
Symptomatic remission was defined as having achieved a Mayo stool frequency subscore of 0 or 1 and a rectal bleeding subscore of 0.

### As-Observed Analysis Approach

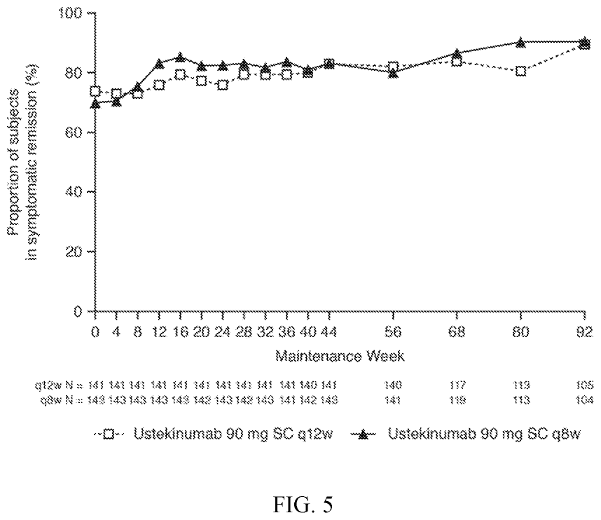

At Week 44, 83.0% and 83.2% of subjects in the ustekinumab q12w and q8w groups, respectively, were in symptomatic remission (Table 14).
Over time, the proportions of subjects in symptomatic remission were sustained from Week 44 through Week 92 in the ustekinumab q12w and q8w groups (FIG. 5). At Week 92, the proportions of subjects in symptomatic remission were 89.5% and 90.4% in the ustekinumab q12w and q8w groups, respectively.
The proportions of subjects in symptomatic remission were sustained from Week 44 to Week 92 in the ustekinumab q12w and q8w groups among the biologic-naïve, biologic-nonfailure, and biologic-failure populations.

### ITT Analysis Approach

Among randomized subjects in maintenance who were treated in the LTE, the proportions of subjects in symptomatic remission at Week 92 were 65.2% and 65.0% in the ustekinumab q12w and q8w groups, respectively.
The proportions of subjects who achieved symptomatic remission at each time point from Week 44 through Week 92 were consistently greater across the ustekinumab q12w and q8w groups in the biologic-naïve and biologic-nonfailure populations compared with the biologic-failure population, with similar proportions observed in the biologic-naïve and biologic-nonfailure populations.

### Maintenance of Symptomatic Remission

The proportions of subjects from the ustekinumab q12w and q8w groups who had achieved symptomatic remission at maintenance baseline were 73.8% and 69.9%, respectively. Among these subjects:
76.0% and 72.0%, respectively, maintained symptomatic remission at Week 92
73.1% and 66.0%, respectively, maintained symptomatic remission at both Week 44 and Week 92
   At Week 44, 83.0% and 83.2% of subjects in the ustekinumab q12w and q8w groups, respectively, were in symptomatic remission. Among these subjects, 72.6% and 70.6%, respectively, maintained symptomatic remission at Week 92.
   Maintenance of symptomatic remission was also assessed based on subjects who had achieved clinical remission at maintenance baseline or at Week 44. The proportions of subjects from the ustekinumab q12w and q8w groups who had achieved clinical remission at maintenance baseline were 24.8% and 22.4%, respectively. Among these subjects, 80.0% and 68.8%, respectively, were in symptomatic remission at both Week 44 and Week 92.
   The proportions of subjects from the ustekinumab q12w and q8w groups who had achieved clinical remission at Week 44 were 46.1% and 52.4%, respectively. Among these subjects, 75.4% and 69.3%, respectively, were in symptomatic remission at Week 92.

### Partial Mayo Remission

### Partial Mayo Remission From Week 44 Through Week 92

### As-Observed Analysis Approach

Using the partial Mayo score to assess remission, the proportions of subjects in partial Mayo remission (i.e., a partial Mayo score ≤2) at Week 44 were similar across ustekinumab treatment groups (83.0% and 84.6% of subjects in the ustekinumab q12w and q8w groups, respectively.
Over time, the proportions of subjects in partial Mayo remission were sustained from Week 44 through Week 92 in the ustekinumab q12w and q8w groups. At Week 92, the proportions of subjects in partial Mayo remission were 91.4% and 91.3% in the ustekinumab q12w and q8w groups, respectively.
The proportions of subjects in partial Mayo remission were sustained from Week 44 to Week 92 in the ustekinumab q12w and q8w groups among the biologic-naïve, biologic-nonfailure, and biologic-failure populations.

### ITT Analysis Approach

Among randomized subjects in maintenance who were treated in the LTE, the proportions of subjects in partial Mayo remission at Week 92 were 66.7% and 65.7% in the ustekinumab q12w and q8w groups, respectively.
The proportions of subjects who achieved partial Mayo remission at each time point from Week 44 through Week 92 were consistently greater across the ustekinumab q12w and q8w groups in the biologic-naïve and biologic-nonfailure populations compared with the biologic-failure population, with similar proportions observed in the biologic-naïve and biologic-nonfailure populations.

### Maintenance of Partial Mayo Remission

The proportions of subjects from the ustekinumab q12w and q8w groups who had achieved partial Mayo remission at maintenance baseline were 68.8% and 70.6%, respectively. Among these subjects, 71.1% and 69.3%, respectively, maintained partial Mayo remission at both Week 44 and Week 92.
At Week 44, 83.0% and 84.6% of subjects in the ustekinumab q12w and q8w groups, respectively, were in partial Mayo remission. Among these subjects, 73.5% and 71.1%, respectively, maintained partial Mayo remission at Week 92.
Maintenance of partial Mayo remission was also assessed based on subjects who had achieved clinical remission at maintenance baseline or at Week 44. At maintenance baseline, 24.8% and 22.4% of subjects in the ustekinumab q12w and q8w groups, respectively, were in clinical remission. Among these subjects, 80.0% and 71.9%, respectively, were in partial Mayo remission at both Week 44 and Week 92.
At Week 44, 46.1% and 52.4% of subjects in the ustekinumab q12w and q8w groups, respectively, had achieved clinical remission. Among these subjects, 75.4% and 69.3%, respectively, were in partial Mayo remission at Week 92.

### Partial Mayo Score

### Partial Mayo Score Over Time

At Week 92, the majority of subjects from the ustekinumab q12w and q8w groups had all 3 subscores of the partial Mayo score (91.5% and 94.4%, respectively). The remaining subjects (12 [8.5%] and 8 [5.6%] subjects in the ustekinumab q12w and q8w groups, respectively) were missing all 3 subscores of the partial Mayo score at Week 92; most of these subjects discontinued study agent prior to Week 92 (10 and 5 subjects in the ustekinumab q12w and q8w groups, respectively). After accounting for treatment failure, 4 and 6 subjects in the ustekinumab q12w and q8w groups, respectively, were missing all 3 subscores of the partial Mayo score at Week 92.

### As-Observed Analysis Approach

At maintenance baseline, the mean partial Mayo scores were 1.9 in both the ustekinumab q12w and q8w groups.
Over time through Week 92, the partial Mayo scores observed at maintenance baseline were generally maintained in the ustekinumab q12w and q8w groups. At Week 92, the mean changes from maintenance baseline in partial Mayo scores for the ustekinumab q12w and q8w groups were −0.8 and −1.0, respectively.

### ITT Analysis Approach

Results based on the ITT analysis approach were generally consistent with those based on the as-observed analysis approach.

### Mayo Rectal Bleeding and Mayo Stool Frequency Subscores Over Time

All Data Presented from this Section Through Section 0 Used the ITT Analysis Approach for the Randomized Population.
The proportions of subjects with a Mayo rectal bleeding subscore of 0 (indicating inactive disease) were comparable in the ustekinumab q12w and q8w groups at maintenance baseline (87.2% and 84.6%, respectively). At Week 92, the proportions of subjects with a Mayo rectal bleeding subscore of 0 were 70.2% and 68.5% in the ustekinumab q12w and q8w groups, respectively.
The proportions of subjects with a Mayo stool frequency subscore of 0 or 1 (indicating inactive or mild disease) were comparable in the ustekinumab q12w and q8w groups at maintenance baseline (80.9% and 80.4%, respectively). At Week 92, the proportions of subjects with a Mayo stool frequency subscore of 0 or 1 were 66.0% and 67.8% in the ustekinumab q12w and q8w groups, respectively.

### Absolute Stool Number Over Time

At maintenance baseline, the mean absolute stool numbers in the ustekinumab q12w and q8w groups were 2.8 and 2.7, respectively, having decreased by at least 3 from induction baseline. Over time, subjects in the ustekinumab q12w and q8w groups maintained their improvement in absolute stool numbers observed at maintenance baseline. At Week 44, the mean absolute stool numbers were 2.4 and 2.3 in the ustekinumab q12w and q8w groups, respectively; at Week 92, the mean absolute stool numbers were 3.4 and 3.2, respectively.
The proportions of subjects with an absolute stool number ≤3 at maintenance baseline were 68.8% and 63.6% in the ustekinumab q12w and q8w groups, respectively. At Week 44, the proportions of subjects with absolute stool numbers ≤3 were 78.0% and 80.4% in the ustekinumab q12w and q8w groups, respectively, and at Week 92, the proportions of subjects were 61.0% and 59.4%, respectively.

### Corticosteriod Endpoints

### Corticosteroid Use

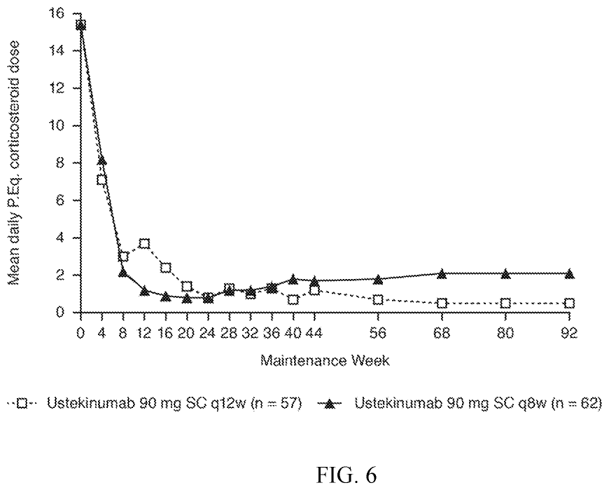

The proportions of randomized subjects who were receiving concomitant corticosteroids (excluding budesonide and beclomethasone dipropionate) at maintenance baseline in the ustekinumab q12w and q8w groups were 40.4% and 43.4%, respectively. Among these subjects:
The mean daily prednisone-equivalent (P.Eq.) corticosteroid dose (excluding budesonide and beclomethasone dipropionate) at maintenance baseline was the same among subjects in both ustekinumab treatment groups (15.4 mg/day). At Week 44, the mean daily doses in the ustekinumab q12w and q8w groups were 1.2 mg/day and 1.7 mg/day, respectively. By Week 92, the mean daily doses were 0.5 mg/day and 2.1 mg/day, respectively.
At Week 92, the mean decreases from maintenance baseline in the average daily P.Eq. doses were 11.3 mg/day and 8.8 mg/day for the ustekinumab q12w and q8w groups, respectively.
A plot of the mean average daily P.Eq. corticosteroid dose (excluding budesonide and beclomethasone dipropionate) through Week 92 is provided in FIG. 6.
   Abbreviations: P.Eq.=prednisone-equivalent; q8w=every 8 weeks; q12w=every 12 weeks; SC=subcutaneous
   The change from Week 44 in the average daily P.Eq. corticosteroid dose (excluding budesonide and beclomethasone dipropionate) from Week 56 through Week 92 among subjects who were receiving corticosteroids other than budesonide and beclomethasone dipropionate at Week 44 is presented.
   Among subjects receiving concomitant corticosteroids (including budesonide and beclomethasone dipropionate) at maintenance baseline, the proportions who were not receiving concomitant corticosteroids at Week 92 were 91.2% and 94.4% in the ustekinumab q12w and q8w groups, respectively.

TABLE 10
Number of Subjects Who Were Not Receiving Concomitant Corticosteroids at Week
92 Among Subjects Who Were Receiving Concomitant Corticosteroids at Maintenance Baseline;
Randomized Subjects in Maintenance Who Were Treated in the Long-Term Extension
(CNTO1275UCO3001).

| | | Ustekinumab | | |
|---|---|---|---|---|
| | Placebo SC ^{a,b} | 90 mg SC q12w ^{b} | 90 mg SC q8w ^{b} | Combined |
| Analysis Set: randomized subjects in | 115 | 141 | 143 | 284 |
| maintenance who were treated in the long- | | | | |
| term extension | | | | |
| Subjects who were receiving concomitant | 54 (47.0%) | 68 (48.2%) | 71 (49.7%) | 139 (48.9%) |
| corticosteroids at maintenance baseline | | | | |
| Week 92 | 44 (81.5%) | 62 (91.2%) | 67 (94.4%) | 129 (92.8%) |
| Subjects not receiving concomitant | | | | |
| corticosteroids ^{c, d, e} | | | | |

Abbreviations: AE = adverse event; IV = intravenous; q8w = every 8 weeks; q12w = every 12 weeks; SC = subcutaneous; UC = ulcerative colitis
^{a} Subjects who were in clinical response to ustekinumab IV induction dosing and were randomized to placebo SC on entry into this maintenance study.
^{b} Randomized group at maintenance Week 0 regardless if subjects had a dose adjustment during the long-term extension.
^{c} Subjects who had an ostomy or colectomy, or discontinued study agent due to lack of therapeutic effect or due to an AE of worsening of UC, or had a dose adjustment (only occurred from Week 56 onward) prior to the Week 92 visit were considered to be receiving concomitant corticosteroids at Week 92.
^{d} Subjects who had a missing value in corticosteroid use at Week 92 had their last available value carried forward.
^{e} Denominator is the number of subjects who were receiving concomitant corticosteroids at maintenance baseline.

### Corticosteroid-Free Symptomatic Remission

The proportions of randomized subjects in the ustekinumab q12w and q8w groups treated during the LTE who were in symptomatic remission and not receiving corticosteroids at Week 92 were 63.8% and 64.3%, respectively (Table 11).
Among subjects receiving corticosteroids at maintenance baseline, the proportions in symptomatic remission and not receiving corticosteroids at Week 92 were consistent with those of the randomized population.

TABLE 11
Number of Subjects in Symptomatic Remission and Not Receiving Corticosteroids at
Week 92; Randomized Subjects in Maintenance Who Were Treated in the Long-Term Extension
(CNTO1275UCO3001).

| | Placebo SC ^{a, b} | 90 mg SC q12w ^{b} | 90 mg SC q8w ^{b} | Combined |
|---|---|---|---|---|
| Analysis Set: randomized | 115 | 141 | 143 | 284 |
| subjects in maintenance who | | | | |
| were treated in the long-term | | | | |
| extension | | | | |
| Week 92 | 25 (21.7%) | 90 (63.8%) | 92 (64.3%) | 182 (64.1%) |
| Subjects in symptomatic | | | | |
| remission and not receiving | | | | |
| corticosteroids at Week 92 ^{c ,d, e, f} | | | | |

Abbreviations: AE = adverse event; IV = intravenous; q8w = every 8 weeks; q12w = every 12 weeks; SC = subcutaneous; UC = ulcerative colitis
^{a} Subjects who were in clinical response to ustekinumab IV induction and were randomized to placebo SC on entry into the maintenance study.
^{b} Randomized group at maintenance Week 0 regardless of whether subjects had a dose adjustment during the long-term extension.
^{c} Symptomatic remission is defined as a stool frequency subscore of 0 or 1 and a rectal bleeding subscore of 0.
^{d} Subjects who had both stool frequency and rectal bleeding subscores missing at a visit were considered not to be in symptomatic remission for that visit.
^{e} Subjects who had an ostomy or colectomy, or discontinued study agent due to lack of therapeutic effect or due to an AE of worsening of UC, or had a dose adjustment (only occurred from Week 56 onward) prior to Week 92 were considered not to be in symptomatic remission.
^{f} Subjects who had a missing value in corticosteroid use had their last value carried forward.

### Corticosteroid-Free Partial Mayo Remission

Among randomized subjects treated during the LTE, the proportions in partial Mayo remission (ie, a partial Mayo score ≤2) and not receiving corticosteroids at Week 92 were 65.2% and 65.0% in the ustekinumab q12w and q8w groups, respectively.
Among subjects receiving corticosteroids at maintenance baseline, the proportions in partial Mayo remission and not receiving corticosteroids at Week 92 were consistent with those of the randomized population.

### Inflammatory Biomarkers

### C-Reactive Protein

Change from Baseline in CRP
At maintenance baseline, median CRP concentrations were 1.5 mg/L and 1.8 mg/L in the ustekinumab q12w and q8w groups, respectively. Over time through Week 92, the median CRP concentrations at maintenance baseline were generally maintained. At Week 92, the median changes from maintenance baseline in CRP concentrations were 0.1 mg/L and 0.0 mg/L for the ustekinumab q12w and q8w groups, respectively.
Normalization of CRP Among Subjects with Abnormal C-Reactive Protein at Induction Baseline
At induction baseline, the proportions of subjects with abnormal CRP (>3 mg/L) were 49.6% and 57.3% in the ustekinumab q12w and q8w groups, respectively. Among these subjects, CRP normalization (≤3 mg/L) at maintenance baseline was reported in 51.4% and 46.3%, respectively. From Week 44 through Week 92, the proportions of subjects with normalized CRP were generally maintained in both ustekinumab groups. At Week 92, the proportions of subjects with normalized CRP were 47.1% and 40.2% in the ustekinumab q12w and q8w groups, respectively.

### Fecal Lactoferrin

Change from Baseline in Fecal Lactoferrin
At maintenance baseline, median fecal lactoferrin concentrations were 37.9 μg/g and 50.0 μg/g in the ustekinumab q12w and q8w groups, respectively. Over time through Week 92, the median fecal lactoferrin concentrations at maintenance baseline were generally maintained. At Week 92, the median changes from maintenance baseline in fecal lactoferrin concentrations were −1.1 μg/g and −12.2 μg/g, respectively.
Normalization of Fecal Lactoferrin Among Subjects with Abnormal Fecal Lactoferrin at Induction Baseline
At induction baseline, the proportion of subjects with abnormal fecal lactoferrin (>7.24 μg/g) was comparable in both ustekinumab treatment groups (89.4% and 90.2% in the q12w and q8w groups, respectively). Of these subjects, normalization of fecal lactoferrin levels at maintenance baseline was reported in 25.4% and 14.7%, respectively. From Week 44 through Week 92, the proportions of subjects with normalized fecal lactoferrin were generally maintained in both ustekinumab groups. At Week 92, 32.5% and 36.4% of subjects in the ustekinumab q12w and q8w groups, respectively, reported normalization of fecal lactoferrin.

### Fecal Calprotectin

Change from Baseline in Fecal Calprotectin Concentration
At maintenance baseline, median fecal calprotectin concentrations were 431.0 mg/kg and 450.5 mg/kg in the ustekinumab q12w and q8w groups, respectively. Over time through Week 92, the median fecal calprotectin concentrations at maintenance baseline were generally maintained. At Week 92, the median changes from maintenance baseline in fecal calprotectin concentrations were −79.5 mg/kg and −94.5 mg/kg, respectively.
Normalization of Fecal Calprotectin Among Subjects with Abnormal Fecal Calprotectin at Induction Baseline
At induction baseline, the proportions of subjects with abnormal fecal calprotectin (>250 mg/kg) were 78.0% and 80.4%, in the ustekinumab q12w and q8w groups, respectively. Among these subjects, normalization of fecal calprotectin levels at maintenance baseline was reported in 28.2% and 28.7% of subject in the ustekinumab q12w and q8w groups, respectively. From Week 44 through Week 92, the proportions of subjects with normalized fecal calprotectin were generally maintained in both ustekinumab groups. At Week 92, 43.6% and 42.6% of subjects in the ustekinumab q12w and q8w groups, respectively, reported normalization of fecal calprotectin.

### Health-Related Quality of Life

### IBDQ

Change from Baseline in Total IBDQ and Each Dimension Score
At induction baseline, the median total IBDQ scores were the same in the ustekinumab q12w and q8w groups (126.0).
At maintenance baseline, the median total IBDQ scores were similar in both ustekinumab treatment groups (181.0 and 175.0 in the ustekinumab q12w and q8w groups, respectively). Over time from Week 44 through Week 92, the median IBDQ scores were generally maintained in the ustekinumab q12w and q8w groups. At Week 92, the median changes from maintenance baseline in the total IBDQ score were 2.0 and 5.0 in the ustekinumab q12w and q8w groups, respectively.
At maintenance baseline, the median IBDQ dimension scores were similar in both ustekinumab treatment groups for each of the 4 dimensions (bowel, emotional, systemic, and social). Over time through Week 92, for each of the 4 dimension scores, the improvements observed at maintenance baseline were maintained in the ustekinumab q12w and q8w groups.
A ≥16-point Improvement from Induction Baseline in the Total IBDQ Score
At Week 92, the proportions of subjects with a ≥16-point improvement from induction baseline in the total IBDQ score were 66.7% and 59.4% in the ustekinumab q12w and q8w groups, respectively.
At maintenance baseline, the proportions of subjects who had a ≥16-point improvement from induction baseline in the total IBDQ score were 88.7% and 87.4% in the ustekinumab q12w and q8w groups, respectively. Among these subjects, the proportions who maintained their 16-point improvement at Week 92 were 68.0% and 61.6% in the ustekinumab q12w and q8w groups, respectively, while 66.4% and 56.8%, respectively, maintained their ≥16-point improvement at both Week 44 and Week 92.
At Week 44, the proportions of subjects with a ≥16-point improvement from induction baseline in the total IBDQ score were 92.2% and 88.8% in the ustekinumab q12w and q8w groups, respectively. Of these subjects, 66.9% and 56.7% of subjects in the ustekinumab q12w and q8w groups, respectively, maintained the 16-point improvement at both Week 68 and Week 92.

### IBDQ Remission

At maintenance baseline, the proportions of subjects who achieved IBDQ remission (IBDQ≥170) were 61.7% and 57.3% in the ustekinumab q12w and q8w groups, respectively. At Week 92, the proportions of subjects who achieved IBDQ remission were 59.6% and 51.7% in the ustekinumab q12w and q8w groups, respectively.
Among subjects in IBDQ remission at maintenance baseline, the proportions who maintained their remission at Week 92 were 74.7% and 59.8% in the ustekinumab q12w and q8w groups, respectively, while 70.1% and 54.9%, respectively, maintained remission at both Week 44 and Week 92.
At Week 44, the proportions of subjects who achieved IBDQ remission were 74.5% and 75.5% in the ustekinumab q12w and q8w groups, respectively. Among subjects with IBDQ remission at Week 44, 66.7% and 54.6% of subjects in the ustekinumab q12w and q8w groups, respectively, maintained remission at both Week 68 and Week 92.

### SF-36

Change from Baseline in SF-36 Physical Component Summary and Mental Component Summary Scores
At induction baseline, the median SF-36 PCS and MCS scores were similar across ustekinumab treatment groups and were below 50 (the United States general population norm score), indicating significant impairment in the subjects' general health (the median PCS scores in the ustekinumab q12w and q8w groups were 43.5 and 43.9, respectively, and the median MCS scores were 41.3 and 39.4, respectively).
At maintenance baseline, the median SF-36 PCS and MCS scores were similar in both ustekinumab treatment groups (median PCS scores of 51.4 and 51.3 in the ustekinumab q12w and q8w groups, respectively; median MCS score of 49.4 in both the ustekinumab q12w and q8w groups).
Over time through Week 92, the median SF-36 PCS scores were maintained in the ustekinumab q12w group and increased (improved) in the ustekinumab q8w group. Median SF-36 MCS scores were maintained in the ustekinumab q12w and q8w groups. At Week 92, the median changes from maintenance baseline in SF-36 PCS scores were 0.0 and 1.4 in the ustekinumab q12w and q8w groups, respectively, and the median changes in SF-36 MCS scores were 0.1 and 0.0, respectively.
A ≥5-point Improvement from Induction Baseline in the SF-36 Physical Component Score
At Week 92, the proportions of subjects who had a ≥5-point improvement from induction baseline in the SF-36 PCS score were 51.8% and 48.3% in the ustekinumab q12w and q8w groups, respectively.
At maintenance baseline, the proportions of subjects who had a ≥5-point improvement from induction baseline in the SF-36 PCS score were 63.8% and 54.5% in the ustekinumab q12w and q8w groups, respectively. Among these subjects, the proportions who maintained their 5-point improvement at Week 92 were 65.6% and 60.3% in the ustekinumab q12w and q8w groups, respectively, while 62.2% and 57.7%, respectively, maintained their ≥5-point improvement at both Week 44 and Week 92.
At Week 44, the proportions of subjects in the ustekinumab q12w and q8w groups who had a ≥5-point improvement from induction baseline in the SF-36 PCS score were 69.5% and 65.7%, respectively. Among these subjects, 61.2% and 56.4% of the ustekinumab q12w and q8w groups, respectively, had a 5-point improvement at both Week 68 and Week 92.
A ≥5-point Improvement from Induction Baseline in the SF-36 Mental Component Score
At Week 92, the proportions of subjects who had a ≥5-point improvement from induction baseline in the SF-36 MCS score were 49.6% and 40.6% in the ustekinumab q12w and q8w groups, respectively.
At maintenance baseline, the proportions of subjects who had a ≥5-point improvement from induction baseline in the SF-36 MCS score were 53.9% and 55.9% in the ustekinumab q12w and q8w groups, respectively. Among these subjects, the proportions who maintained their 5-point improvement at Week 92 were 71.1% and 48.8% in the ustekinumab q12w and q8w groups, respectively, while 65.8% and 43.8%, respectively, maintained their ≥5-point improvement at both Week 44 and Week 92.
At Week 44, the proportions of subjects in the ustekinumab q12w and q8w groups who had a ≥5-point improvement from induction baseline in the SF-36 MCS score were 58.9% and 64.3%, respectively. Among these subjects, 63.9% and 44.6% of the ustekinumab q12w and q8w groups, respectively, had a ≥5-point improvement at both Week 68 and Week 92.

### Clinical Efficacy in Subjects Who had a Dose Adjustment

Subjects in the main analysis population (i.e., those who were randomized at maintenance Week 0) whose UC disease activity worsened, based on the clinical judgment of the investigator, were eligible for a dose adjustment. Eligible subjects randomized to placebo or ustekinumab q12w had a dose adjustment to a ustekinumab q8w regimen while subjects randomized to ustekinumab q8w remained on the q8w regimen (sham dose adjustment). Among randomized subjects treated in the LTE, 46.1% (53 subjects), 28.4% (40 subjects), and 25.9% (37 subjects) from the placebo, ustekinumab q12w, and ustekinumab q8w groups, respectively, had a dose adjustment to ustekinumab q8w during the LTE.
Subjects who had a dose adjustment were assessed 16 weeks after the dose adjustment visit to determine if benefit was achieved from the dose adjustment. Interpretation of these data is limited by the small sample sizes.

### Clinical Efficacy

### Symptomatic Remission

Among the subjects in the ustekinumab q12w→q8w and ustekinumab q8w→q8w groups who had data at least 16 weeks after dose adjustment, 55.0% (11 of 20 subjects) and 64.3% (18 of 28 subjects), respectively, were in symptomatic remission at the time of dose adjustment, and 70.0% (14 of 20 subjects) and 71.4% (20 of 28 subjects), respectively, were in symptomatic remission at the first visit at least 16 weeks after dose adjustment. The majority of subjects were in symptomatic remission at the time of dose adjustment; this may be due to the fact that dose adjustment was based on the clinical judgment of the investigator and no other pre-specified criteria (e.g., clinical flare based on the partial Mayo score as was applied through Week 44 of the maintenance study).
Among the subjects who were not in symptomatic remission at the time of dose adjustment and had data at least 16 weeks after dose adjustment in the ustekinumab q12w→q8w and ustekinumab q8w→q8w groups, 44.4% (4 of 9 subjects) and 60.0% (6 of 10 subjects), respectively, were in symptomatic remission at the first visit at least 16 weeks after dose adjustment. However, it should be noted that the number of subjects in this analysis was limited.

### Partial Mayo Remission

Among the subjects in the ustekinumab q12w→q8w and ustekinumab q8w→q8w groups who had data at least 16 weeks after dose adjustment, 55.0% (11 of 20 subjects) and 60.7% (17 of 28 subjects), respectively, were in partial Mayo remission at the time of dose adjustment, and 70.0% (14 of 20 subjects) and 67.9% (19 of 28 subjects), respectively, were in partial Mayo remission at the first visit at least 16 weeks after dose adjustment.
Among the subjects who were not in partial Mayo remission at the time of dose adjustment and had data at least 16 weeks after dose adjustment in the ustekinumab q12w→q8w and ustekinumab q8w→q8w groups, 44.4% (4 of 9 subjects) and 63.6% (7 of 11 subjects), respectively, were in partial Mayo remission at the first visit at least 16 weeks after dose adjustment. However, it should be noted that the number of subjects in this analysis was limited.

### Partial Mayo Score

Among the subjects in the ustekinumab q12w→q8w and ustekinumab q8w→q8w groups who had data at least 16 weeks after dose adjustment, the mean partial Mayo scores were 2.4 and 2.5, respectively, at the time of dose adjustment, and 2.0 and 2.0, respectively, at the first visit at least 16 weeks after dose adjustment.

### Inflammatory Biomarkers

### C-Reactive Protein

Among the subjects in the ustekinumab q12w→q8w and ustekinumab q8w→q8w groups who had data at least 16 weeks after dose adjustment, the median CRP concentrations were 3.1 and 2.3 mg/L, respectively, at the time of dose adjustment, and 2.6 and 1.8 mg/L, respectively, at the first visit at least 16 weeks after dose adjustment.
Fecal Lactoferrin Among the subjects in the ustekinumab q12w→q8w and ustekinumab q8w→q8w groups who had data at least 16 weeks after dose adjustment, the median fecal lactoferrin concentrations were 38.2 and 30.7 μg/g, respectively, at the time of dose adjustment, and 52.2 and 16.2 μg/g, respectively, at the first visit at least 16 weeks after dose adjustment.

### Fecal Calprotectin

Among the subjects in the ustekinumab q12w→q8w and ustekinumab q8w→q8w groups who had data at least 16 weeks after dose adjustment, the median fecal calprotectin concentrations were 604.5 and 414.5 mg/kg, respectively, at the time of dose adjustment, and 850.0 and 396.5 mg/kg, respectively, at the first visit at least 16 weeks after dose adjustment.

### Dose Adjustment as a Treatment Strategy

To reflect clinical practice where treatments are optimized either through increases in dose or dosing frequency, the data were alternatively evaluated through an analytic approach that treats dose adjustment as a treatment strategy. In this analysis approach, the dose adjustment treatment failure criterion was suspended and subjects starting on q12w or q8w remained in their randomized treatment group whether or not a subsequent dose adjustment occurred.

### Symptomatic Remission

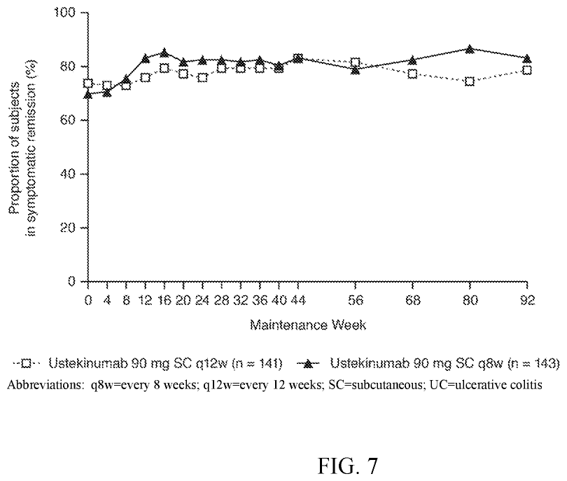

When examined using the dose-adjustment-as-a-treatment-strategy analysis approach, the proportions of subjects in symptomatic remission were sustained from Week 44 through Week 92 in the ustekinumab q12w and q8w groups (FIG. 7).

### Partial Mayo Remission

When examined using the dose-adjustment-as-a-treatment-strategy analysis approach, the proportions of subjects in partial Mayo remission were sustained from Week 44 through Week 92 in the ustekinumab q12w and q8w groups.

### Efficacy in Subjects Resuming Ustekinumab After Treatment Interruption

Among the subjects who were in clinical response to ustekinumab IV induction, randomized to placebo at maintenance baseline, and treated during the LTE, a total of 42 subjects had a dose adjustment to ustekinumab q8w during the LTE and had data at least 16 weeks after dose adjustment.
The results presented below suggest that in the subset of subjects who responded to the ustekinumab IV induction dose but delayed initiation of the SC ustekinumab maintenance therapy, benefit can be regained. However, it should be noted that the number of subjects in this group was limited (42 subjects total).

### Clinical Efficacy Endpoints

### Symptomatic Remission

Among the subjects in the placebo→ustekinumab q8w group who had data at least 16 weeks after dose adjustment, 40.5% (17 of 42 subjects) were in symptomatic remission at the time of dose adjustment, and 71.4% (30 of 42 subjects) were in symptomatic remission at the first visit at least 16 weeks after dose adjustment.
Among the subjects who were not in symptomatic remission at the time of dose adjustment and had data at least 16 weeks after dose adjustment in the placebo→ustekinumab q8w group, 64.0% (16 of 25 subjects) were in symptomatic remission at the first visit at least 16 weeks after dose adjustment.

### Partial Mayo Remission

Among the subjects in the placebo→ustekinumab q8w group who had data at least 16 weeks after dose adjustment, 40.5% (17 of 42 subjects) were in partial Mayo remission at the time of dose adjustment, and 76.2% (32 of 42 subjects) were in partial Mayo remission at the first visit at least 16 weeks after dose adjustment.
Among the subjects who were not in partial Mayo remission at the time of dose adjustment and had data at least 16 weeks after dose adjustment in the placebo→ustekinumab q8w group, 80.0% (20 of 25 subjects) were in partial Mayo remission at the first visit at least 16 weeks after dose adjustment.

### Partial Mayo Score

Among the subjects in the placebo→ustekinumab q8w group who had data at least 16 weeks after dose adjustment, the mean partial Mayo score was 3.2 at the time of dose adjustment and 1.5 at the first visit at least 16 weeks after dose adjustment.

### Inflammatory Biomarkers

Among subjects in the placebo→ustekinumab q8w group who had data at least 16 weeks after dose adjustment, the median inflammatory biomarker concentrations at the time of dose adjustment and at the first visit at least 16 weeks after dose adjustment, respectively, are as follows:
C-reactive protein: 3.6 mg/L, 2.0 mg/L
Fecal lactoferrin: 128.9 μg/g, 28.3 μg/g
Fecal calprotectin: 1016.5 mg/kg, 355.0 mg/kg
   Nonrandomized Subjects in Maintenance Who Were Treated in the Long-Term Extension Efficacy as determined by clinical efficacy measures (symptomatic remission, partial Mayo remission, partial Mayo scores, and corticosteroid-free remission [symptomatic and partial Mayo]), change in inflammatory biomarker levels (CRP, fecal lactoferrin, and fecal calprotectin), and health-related quality of life measures (IBDQ and SF-36) are each summarized for nonrandomized subjects.
   The data presented in this section are from subjects in the ustekinumab induction delayed-responder group (n=116) who were treated in the LTE. These subjects were not in clinical response to IV ustekinumab at induction Week 8 but were in clinical response at induction Week 16 after receiving ustekinumab 90 mg SC at induction Week 8. Subjects in this group received ustekinumab 90 mg SC q8w during maintenance (through Week 44) and during the LTE (Week 44 through Week 96).
   The placebo induction responder group (n=73) consists of subjects who achieved clinical response to placebo at induction Week 8 and were treated in the LTE. These subjects were enrolled into the maintenance study to maintain the blind and continued treatment during the LTE. Subjects in this group received placebo SC during the LTE and the data are summarized in the same tables as listed for the ustekinumab induction delayed-responder group.

### Clinical Efficacy

### Symptomatic Remission

### Symptomatic Remission From Week 44 Through Week 92

### As-Observed Analysis Approach

At Week 44, 74.1% of subjects in the ustekinumab induction delayed-responder group were in symptomatic remission. Over time, the proportion was sustained, with 81.4% in symptomatic remission at Week 92.

### ITT Analysis Approach

Results from ITT analysis approach were similar to those of the as-observed analyses above. At Week 92, 79.3% of subjects in the ustekinumab induction delayed-responder group were in symptomatic remission (Table 12).
Of note, dose adjustment was not part of the treatment failure rule in this analysis since nonrandomized subjects were not eligible for dose adjustment in the LTE. When comparing this analysis with the corresponding analysis treating dose adjustment as a treatment strategy for subjects in the randomized ustekinumab q8w group where the dose adjustment treatment failure criterion was suspended, similar results were observed (Table 12).

TABLE 12
Number of Subjects in Symptomatic Remission at Week 92 From the
Randomized Ustekinumab q8w Group and the Ustekinumab Induction Delayed-Responder
Group with Dose Adjustment Treatment Failure Criterion Suspended; Subjects Who Were
Treated with Ustekinumab 90 mg SC q8w during the Long-Term Extension
(CNTO1275UCO3001).

| | | Nonrandomized subjects |
|---|---|---|
| | Randomized subjects ^{a} | (Delayed responders ^{b} |
| | (ustekinumab q8w) | [ustekinumab q8w]) |
| Analysis Set: subjects in maintenance | 143 | 116 |
| who were treated with ustekinumab 90 mg | | |
| SC q8w during the long-term extension | | |
| Subjects in symptomatic remission at | 119 (83.2%) | 92 (79.3%) |
| Week 92 ^{c, d, e} | | |

Abbreviations: AE = adverse event; q8w = every 8 weeks; q12w = every 12 weeks; SC = subcutaneous; U = ulcerative colitis
^{a} Randomized group at maintenance Week 0 regardless of whether subjects had a dose adjustment during the long-term extension.
^{b} Subjects who were not in clinical response to ustekinumab at induction Week 8 but were in clinical response at induction Week 16 after a SC administration of ustekinumab at induction Week 8.
^{c} Symptomatic remission is defined as a stool frequency subscore of 0 or 1 and a rectal bleeding subscore of 0.
^{d} Subjects who had both stool frequency and rectal bleeding subscores missing at a visit were considered not to be in symptomatic remission for that visit.
^{e} Subjects who had an ostomy or colectomy, or discontinued study agent due to lack of therapeutic effect or due to an AE of worsening of UC, prior to the designated visit were considered not to be in symptomatic remission.

The proportions of subjects in symptomatic remission were sustained from Week 44 to Week 92 in the ustekinumab induction delayed-responder group among the biologic-naïve, biologic-nonfailure, and biologic-failure populations.

### Maintenance of Symptomatic Remission

Among subjects in the ustekinumab induction delayed-responder group who were treated in the LTE, the proportion of subjects who achieved symptomatic remission at maintenance baseline was 63.8%. Among these subjects:
87.8% maintained symptomatic remission at Week 92
82.4% maintained symptomatic remission at both Week 44 and Week 92
   At Week 44, 74.1% of subjects in the ustekinumab induction delayed-responder group were in symptomatic remission. Among these subjects, 89.5% maintained symptomatic remission at Week 92.
   Maintenance of symptomatic remission was also assessed based on subjects who had achieved clinical remission at maintenance baseline or at Week 44. The proportion of subjects from the ustekinumab induction delayed-responder group treated in the LTE who were in clinical remission at maintenance baseline was 12.9%. Among these subjects, 100.0% (15 subjects) were in symptomatic remission at both Week 44 and Week 92.
   At Week 44, 38.8% of subjects from the ustekinumab induction delayed-responder group had achieved clinical remission. Among these subjects, 95.6% were in symptomatic remission at Week 92.

### Partial Mayo Remission

### Partial Mayo Remission From Week 44 Through Week 92

### As-Observed Analysis Approach

Using the partial Mayo score to assess remission, the proportions of subjects from the ustekinumab induction delayed-responder group treated in the LTE who were in partial Mayo remission (ie, a partial Mayo score ≤2) at Week 44 was 72.4%. Overtime, the proportion of subjects was sustained, with 84.1% of subjects in partial Mayo remission at Week 92.

### ITT Analysis Approach

Results from the ITT analysis approach for the proportion of subjects from the ustekinumab induction delayed-responder group treated in the LTE who were in partial Mayo remission over time through Week 92 were similar to those of the as-observed analysis approach above. At Week 92, the proportion of subjects in partial Mayo remission was 81.9%.
The proportions of subjects in partial Mayo remission were sustained from Week 44 to Week 92 in the ustekinumab induction delayed-responder group among the biologic-naïve, biologic-nonfailure, and biologic-failure populations.

### Maintenance of Partial Mayo Remission

Among subjects in the ustekinumab induction delayed-responder group who were treated in the LTE, the proportion that achieved partial Mayo remission at maintenance baseline was 64.7%. Among these subjects, 82.7% maintained partial Mayo remission at both Week 44 and Week 92.
At Week 44, 72.4% of subjects from the ustekinumab induction delayed-responder group treated in the LTE were in partial Mayo remission. Among these subjects, 92.9% maintained partial Mayo remission at Week 92.
Maintenance of partial Mayo remission was also assessed based on subjects who had achieved clinical remission at maintenance baseline or at Week 44. The proportion of subjects from the ustekinumab induction delayed-responder group treated in the LTE who were in clinical remission at maintenance baseline was 12.9%. Among these subjects, 93.3% were in partial Mayo remission at both Week 44 and Week 92.
At Week 44, 38.8% of subjects from the ustekinumab induction delayed-responder group were in clinical remission. Among these subjects, 93.3% were in partial Mayo remission at Week 92.

### Partial Mayo Score

### Partial Mayo Score Over Time

### As-Observed Analysis Approach

The mean partial Mayo score at maintenance baseline among subjects in the ustekinumab induction delayed-responder group was 2.2. At Week 44, the mean change from maintenance baseline in partial Mayo score among subjects in the ustekinumab induction delayed-responder group was −0.5, and at Week 92, the mean change from maintenance baseline was −0.9.

### ITT Analysis Approach

Results from the ITT analysis approach were generally consistent with those of the as-observed analysis approach.

### Mayo Rectal Bleeding and Mayo Stool Frequency Subscores Over Time

All data presented from this section through Section Partial Mayo Remission From Week 0 Through Week 92 are from an ITT analysis approach for the non-randomized population.
The proportions of subjects from the ustekinumab induction delayed-responder group with a Mayo rectal bleeding subscore of 0 (indicating inactive disease) were maintained over time through Week 92: 81.0% at maintenance baseline, 88.8% at Week 44, and 87.9% at Week 92.
The proportions of subjects from the ustekinumab induction delayed-responder group with a Mayo stool frequency subscore of 0 or 1 (indicating inactive or mild disease) were maintained over time through Week 92: 72.4% at maintenance baseline, 79.3% at Week 44, and 82.8% at Week 92.

### Corticosteriod Endpoints

### Corticosteroid-Free Symptomatic Remission

The proportion of subjects from the ustekinumab induction delayed-responder group who were in symptomatic remission and not receiving corticosteroids at Week 92 was 75.0%.
Among subjects receiving corticosteroids at maintenance baseline, the proportion in symptomatic remission and not receiving corticosteroids at Week 92 was 68.6%.

### Corticosteroid-free Partial Mayo Remission

The proportion of subjects in the ustekinumab induction delayed-responder group who were in partial Mayo remission and not receiving corticosteroids at Week 92 was 77.6%.
Among subjects receiving corticosteroids at maintenance baseline, the proportion in partial Mayo remission and not receiving corticosteroids at Week 92 was 72.5%.

### Inflammatory Biomarkers

### C-Reactive Protein

Change from Baseline in CRP
Among subjects from the ustekinumab induction delayed-responder group, the median CRP concentration at maintenance baseline was 1.9 mg/L. Overtime through Week 92, the median CRP concentration observed at maintenance baseline was generally maintained in the ustekinumab induction delayed-responder group, with a median change from maintenance baseline in CRP concentration at Week 92 of −0.2 mg/L.
Normalization of CRP among Subjects with Abnormal C-reactive Protein at Induction Baseline
At induction baseline, the proportion of subjects from the ustekinumab induction delayed-responder group with abnormal CRP (>3 mg/L) was 68.1%. Among these subjects, CRP normalization (≤3 mg/L) at maintenance baseline was reported in 59.5%. Over time, the proportion of subjects with normalized CRP was generally maintained, with normalized CRP reported in 57.0% of subjects at Week 92.

### Fecal Lactoferrin

Change from Baseline in Fecal Lactoferrin
Among subjects from the ustekinumab induction delayed-responder group, the median fecal lactoferrin concentration at maintenance baseline was 52.58 μg/g. Over time through Week 92, the median fecal lactoferrin concentration observed at maintenance baseline was generally maintained in the ustekinumab induction delayed-responder group, with a median change in fecal lactoferrin concentration from maintenance baseline at Week 92 of −19.07 μg/g.
Normalization of Fecal Lactoferrin Among Subjects with Abnormal Fecal Lactoferrin at Induction Baseline
At induction baseline, the proportion of subjects from the ustekinumab induction delayed-responder group with abnormal fecal lactoferrin (>7.24 μg/g) was 93.1%. Among these subjects, normalization of fecal lactoferrin levels at maintenance baseline was reported in 17.6%. From Week 44 through Week 92, the proportion of subjects with normalization of fecal lactoferrin was generally sustained, with normalized fecal lactoferrin reported in 34.3% of subjects at Week 92.

### Fecal Calprotectin

Change from Baseline in Fecal Calprotectin Concentration
Among subjects from the ustekinumab induction delayed-responder group, the median fecal calprotectin concentration at maintenance baseline was 428.0 mg/kg. Over time through Week 92, the median fecal calprotectin concentration observed at maintenance baseline was generally maintained in the ustekinumab induction delayed-responder group with a median change in fecal calprotectin concentration from maintenance baseline at Week 92 of −113.0 mg/kg.
Normalization of Fecal Calprotectin Among Subjects with Abnormal Fecal Calprotectin at Induction Baseline
At induction baseline, the proportion of subjects with abnormal fecal calprotectin (>250 mg/kg) was 82.8%. Among these subjects, normalization of fecal calprotectin levels at maintenance baseline was reported in 26.0%. Over time, this proportion was maintained, with normalized fecal calprotectin reported in 42.7% of subjects at Week 92.

### Health-Related Quality of Life

### IBDQ

Change from Baseline in Total IBDQ
Among subjects from the ustekinumab induction delayed-responder group, the median total IBDQ score at maintenance baseline was 180.0. Over time through Week 92, the median IBDQ score increased (improved), with a median change from baseline in the total IBDQ score at Week 92 of 10.0.
A ≥16-point Improvement from Induction Baseline in the Total IBDQ Score
At maintenance baseline, 80.2% of subjects from the ustekinumab induction delayed-responder group had a ≥16-point improvement from induction baseline in the total IBDQ score. This proportion was maintained over time, with 80.2% of subjects reporting a ≥16-point improvement from induction baseline in the total IBDQ score at Week 92.

### IBDQ Remission

At maintenance baseline, 62.9% of subjects achieved IBDQ remission (IBDQ≥170). This proportion was maintained over time, with IBDQ remission reported in 69.8% of subjects at Week 92.

### SF-36

Change from Baseline in SF-36 Physical and Mental Component Summary Scores Among subjects in the ustekinumab induction delayed-responder group, the median SF-36 PCS and MCS scores at maintenance baseline were 51.8 and 49.5, respectively. Over time through Week 92, the median SF-36 PCS and MCS scores were maintained, with median changes from maintenance baseline in SF-36 PCS and MCS scores at Week 92 of 1.2 and 1.1, respectively.
A ≥5-point Improvement from Induction Baseline in the SF-36 Physical Component Score
At maintenance baseline, 52.6% of subjects from the ustekinumab induction delayed-responder group had a ≥5-point improvement from induction baseline in the SF-36 PCS score. This proportion was maintained over time, with 61.2% of subjects reporting a ≥5-point improvement from induction baseline in the SF-36 PCS score at Week 92.
A ≥5-point Improvement from Induction Baseline in the SF-36 Mental Component Score
At maintenance baseline, 56.9% of subjects from the ustekinumab induction delayed-responder group had a 5-point improvement from induction baseline in the SF-36 MCS score. This proportion was maintained over time, with 57.8% of subjects reporting a ≥5-point improvement from induction baseline in the SF-36 MCS score at Week 92.

### Selected Analyses Among All Subjects Enrolled at Maintenance Baseline

To assess maintenance of clinical benefit from maintenance Week 0 through Week 92 for all subjects enrolled in the maintenance study, symptomatic remission and partial Mayo remission from maintenance Week 0 through Week 96 were summarized separately for all randomized and nonrandomized subjects at maintenance baseline, regardless of whether subjects were treated in the LTE.

### Symptomatic Remission From Week 0 Through Week 92

### Randomized Subjects

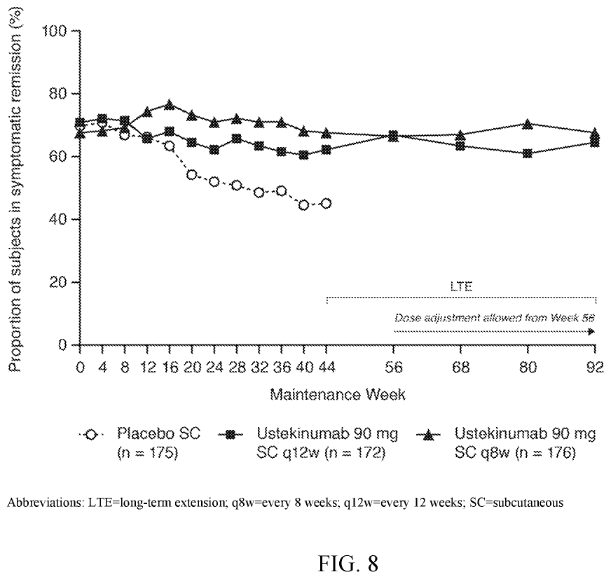

To reflect the clinical practice where treatments are optimized either through increases in dose or dosing frequency, the data presented here focus on an analytic approach that treats dose adjustment as a treatment strategy, where subjects who had a dose adjustment were not considered to be treatment failures. In this analysis, the same treatment failure rules as that used in UCO3001 W44, which included prohibited medication criteria, were applied through Week 44 while treatment failure rules without protocol-prohibited medication changes were applied from Week 44 onward.
The proportions of subjects randomized at maintenance baseline who were in symptomatic remission were sustained from Week 44 to Week 92, with 64.5% (111 subjects) and 67.6% (119 subjects) in the ustekinumab q12w and q8w groups, respectively, at Week 92 (FIG. 8).

### Nonrandomized Subjects

The proportion of subjects in symptomatic remission in the ustekinumab induction delayed-responder group was sustained over time, with 56.1% at Week 0, 51.6% at Week 44, and 58.6% at Week 92.

### Partial Mayo Remission From Week 0 Through Week 92 Randomized Subjects

The proportion of subjects randomized at maintenance baseline who were in partial Mayo remission over time from maintenance baseline through Week 92 was summarized by treatment group.
When dose adjustment was not considered to be a treatment failure, the proportions of subjects who were in partial Mayo remission in the ustekinumab q12w and q8w groups, respectively, over time were as follows:
Week 0: 67.4% and 69.3%
Week 44: 62.2% and 68.8%
Week 92: 66.3% and 67.6%

### Nonrandomized Subjects

The proportion of subjects from the ustekinumab induction delayed-responder group who were in partial Mayo remission was sustained over time, with 56.7% at Week 0, 51.0% at Week 44, and 60.5% at Week 92.

### Efficacy and Pharmacokinetics

The population for efficacy and PK analyses was randomized subjects in this maintenance study who received ustekinumab during the LTE, did not have a dose adjustment, and who had appropriate concentration data through Week 92 of the LTE. Because Week 92 was not a trough concentration timepoint, analysis of the relationship between efficacy at Week 92 and ustekinumab concentration at Week 92 was performed separately for each ustekinumab treatment group. In addition, analyses examining the association between efficacy at Week 92 and average trough ustekinumab concentration (calculated based on respective trough concentration data for each ustekinumab treatment group from Week 24 through Week 88) were also performed.
The relationships between serum ustekinumab concentration and efficacy presented in this report are as follows:
Symptomatic remission at Week 92 versus ustekinumab concentration quartiles at Week 92 or average serum ustekinumab concentration quartiles
Partial Mayo remission at Week 92 versus ustekinumab concentration quartiles at Week 92 or average serum ustekinumab concentration quartiles
Serum CRP concentration and % normalized CRP at Week 92 versus ustekinumab concentration quartiles at Week 92 or average serum ustekinumab concentration quartiles
Fecal calprotectin concentration and % normalized fecal calprotectin at Week 92 versus ustekinumab concentration quartiles at Week 92 or average serum ustekinumab concentration quartiles. Similar analyses were performed for fecal lactoferrin.

### Symptomatic and Partial Mayo Remission

In general, high proportions (≥80%) of subjects were in symptomatic remission and partial Mayo remission in each concentration quartile. Accordingly, no clear exposure-efficacy relationship was observed between serum ustekinumab concentration and these efficacy endpoints in this population of subjects who were considered to have benefited from maintenance treatment.

### Inflammatory Biomarkers

In general, for both ustekinumab SC treatment groups, median CRP at Week 92 decreased with increasing serum ustekinumab concentration quartiles. In the combined ustekinumab treatment group, median CRP was higher in the lowest ustekinumab concentration quartile when compared to the other quartiles. In line with these observations, among subjects with abnormal CRP (>3 mg/L) at induction baseline, the proportions of subjects with normalized CRP at Week 92 increased with increasing serum ustekinumab concentration quartile.
In the respective ustekinumab treatment group, compared to the other quartiles, median fecal calprotectin at Week 92 was lowest in the highest Week 92 ustekinumab concentration quartile. In the combined ustekinumab treatment group, median fecal calprotectin at Week 92 was also lowest in highest average serum trough ustekinumab concentration quartile when compared with the other quartiles. In both ustekinumab treatment groups, among subjects with abnormal fecal calprotectin (>250 mg/kg) at induction baseline, the proportions of subjects with normalized calprotectin at Week 92 increased with increasing serum ustekinumab concentration quartile. Similar patterns were generally observed in the fecal lactoferrin analyses.

### Efficacy and Immunogenicity

The populations for the analyses of efficacy and immunogenicity were randomized subjects in maintenance who were treated in the LTE.
An evaluation of antibody to ustekinumab status through Week 96 versus symptomatic remission and partial Mayo remission at Week 92 was performed to determine the influence of antibodies to ustekinumab on the efficacy of ustekinumab. Because of the limited number of subjects who were positive for antibodies to ustekinumab, these analyses should be interpreted with caution.
In subjects who were randomized in maintenance to ustekinumab and treated in the LTE, the proportions of subjects who were in symptomatic or partial Mayo at Week 92 were comparable between those who were positive and those who were negative for antibodies to ustekinumab. For example, in the ustekinumab q12w group, 80% (4 subjects) of subjects were in symptomatic remission among those who were positive for antibodies compared with 85.4% (82 subjects) among those who were negative. In the ustekinumab q8w group, 80% (4 subjects) of subjects were in partial Mayo remission among those who were positive for antibodies compared with 87.1% (88 subjects) among those who were negative. A similar pattern was observed for partial Mayo remission.

### Efficacy Summary

### Randomized Subjects Treated in the LTE

From Week 44 through Week 92, the proportions of randomized subjects in the ustekinumab q12w and q8w groups in symptomatic remission and the proportions in partial Mayo remission were sustained.
   Sustained efficacy was similarly observed in the biologic-naïve, biologic nonfailure, and biologic-failure populations.
With continued ustekinumab treatment in the LTE, subjects were able to achieve symptomatic remission and partial Mayo remission in the absence of corticosteroids at Week 92.
Continued treatment with ustekinumab enabled patients to eliminate corticosteroids.
With continued ustekinumab treatment from Week 44 through Week 92:
   Reductions in partial Mayo score observed at maintenance baseline were sustained with continued ustekinumab treatment from Week 44 through Week 92; the majority of subjects achieved a Mayo rectal bleeding subscore of 0, a Mayo stool frequency subscore of 0 or 1, and an absolute stool number ≤3.
   The reductions in CRP, fecal lactoferrin, and fecal calprotectin observed at maintenance baseline were sustained from Week 44 through Week 92.
   Improvements in health-related quality of life (IBDQ and SF-36) observed at maintenance baseline were sustained from Week 44 through Week 92.
Some benefit of dose adjustment was observed among randomized subjects treated in the LTE who had a dose adjustment.

### Ustekinumab Induction Delayed Responders Treated in the LTE

Subjects were able to sustain symptomatic remission and partial Mayo remission from Week 44 through Week 92, achieve corticosteroid-free remission at Week 92, sustain reduction in inflammatory biomarkers from Week 44 through Week 92, and sustain improvement in health-related quality of life from Week 44 through Week 92
Clinical benefits observed among these subjects was similar to that observed for randomized subjects treated with ustekinumab q8w in the LTE.

### Efficacy and Pharmacokinetics

In general, high proportions (≥80%) of subjects were in symptomatic remission and partial Mayo remission in each concentration quartile. Accordingly, no clear exposure-efficacy relationship was observed between serum ustekinumab concentration and these efficacy endpoints in this population of subjects who were considered to have benefited from maintenance treatment.

### Efficacy and Immunogenicity

The proportions of randomized subjects in remission at Week 92 were comparable between those who were positive and those who were negative for antibodies to ustekinumab.

### Safety Results

Safety data through Week 44 for randomized subjects and for nonrandomized subjects in maintenance were previously presented in the UCO3001 44W. Summaries of AEs and other safety data in this report are mainly based on data from Week 44 through Week 96. However, key safety summaries from Week 0 of maintenance to Week 96 are also provided.
Safety data from Week 44 through Week 96 are presented for:
All treated subjects, ie, all subjects who were treated in the LTE, including both randomized and nonrandomized subjects, to provide an accounting of all safety events.
Placebo SC
   Randomized subjects from the placebo group (including data up to the time of dose adjustment)
   Nonrandomized subjects from the placebo induction responder group
Ustekinumab 90 mg SC q12w
   Randomized subjects from the ustekinumab q12w group (including data up to the time of dose adjustment)
Ustekinumab 90 mg SC q8w
   Randomized subjects from the ustekinumab q8w group
   Randomized subjects from the placebo group who had a dose adjustment to ustekinumab q8w (including data from the time of dose adjustment onward)
   Randomized subjects from the ustekinumab q12w group who had a dose adjustment to ustekinumab q8w (including data from the time of dose adjustment onward)
   Nonrandomized subjects from the ustekinumab induction delayed-responder group
Combined ustekinumab (ustekinumab 90 mg SC q12w+ustekinumab 90 mg SC q8w)
   Randomized subjects from the ustekinumab q8w group
   Randomized subjects from the ustekinumab q12w group
   Randomized subjects (placebo [including data from the time of dose adjustment onward] or ustekinumab q12w) who had a dose adjustment to ustekinumab q8w
   Nonrandomized subjects from the ustekinumab induction delayed-responder group (treated with ustekinumab q8w)
All treated subjects by randomization status, i.e., all subjects who were treated in the LTE by randomization status:
Randomized subjects (data from Week 44 through Week 96 or up to time of dose adjustment were summarized):
   Placebo SC
   Ustekinumab 90 mg SC q12w
   Ustekinumab 90 mg SC q8w
   Combined ustekinumab
Nonrandomized subjects:
   Placebo induction responders
   Ustekinumab induction delayed responders
   For the purposes of this report, interpretation of data for the nonrandomized subjects focuses on the ustekinumab induction delayed responders.

### Summary of All Adverse Events

### Week 44 Through Week 96

### All Treated Subjects

The average duration of follow-up for subjects in the placebo group (37.1 weeks) was shorter than that in the ustekinumab q12w (44.5 weeks) and q8w (45.3 weeks) groups, largely due to subjects remaining on placebo being discontinued at the time of study unblinding; duration of follow-up was comparable in the ustekinumab groups. To account for the different durations of follow-up across the treatment groups, the incidence of key safety findings per hundred subject-years of follow-up for all treated subjects from Week 44 through Week 96 was summarized.
The number of specified events per hundred subject years of follow-up from Week 44 through Week 96 was generally comparable for subjects treated with ustekinumab as compared with subjects treated with placebo for AEs and SAEs (Table 13). While AEs of infection per 100 subject-years were numerically higher for subjects in the ustekinumab 90 mg q8w group compared with subjects in the placebo and ustekinumab q12w groups, events of serious infection per 100 subject-years were similar across treatment groups.
One subject in the randomized placebo group (who had a dose adjustment to ustekinumab 90 mg q8w during the LTE, receiving only 1 dose) died due to an event of cardiac arrest on Day 573.

TABLE 13
Summary of Key Safety Findings Per Hundred Subject-Years of Follow-Up From
Week 44 Through Week 96; Subjects Who Were Treated in the Long-Term Extension
(CNTO1275UCO3001).

| | | Ustekinumab | | |
|---|---|---|---|---|
| | Placebo | 90 mg SC | | |
| | SC ^{a} | q12w ^{b} | 90 mg SC q8w ^{c} | Combined |
| Analysis Set: subjects who were treated in the long-term | 188 | 141 | 353 | 454 |
| extension | | | | |
| Avg duration of follow-up from Week 44 to Week 96 (weeks) | 37.1 | 44.5 | 45.3 | 49.1 |
| Total subject-years of follow-up from Week 44 to Week 96 | 134.0 | 120.6 | 307.6 | 428.3 |
| Number of specified events per hundred subject-years of | | | | |
| follow-up (95% CI) ^{d} | | | | |
| Adverse events | 267.93 | 223.82 | 268.17 | 255.68 |
| | (240.93, | (197.91, | (250.18, | (240.76, |
| | 297.13) | 252.17) | 287.11) | 271.28) |
| Serious adverse events | 12.69 | 5.80 | 10.73 | 9.34 |
| | (7.39, | (2.33, | (7.38, | (6.67, |
| | 20.31) | 11.96) | 15.06) | 12.72) |
| Infections ^{e} | 80.60 | 81.24 | 90.69 | 88.03 |
| | (66.12, | (65.95, | (80.36, | (79.36, |
| | 97.31) | 99.00) | 101.98) | 97.38) |
| Serious infections ^{e} | 2.99 | 3.32 | 1.95 | 2.33 |
| | (0.81, | (0.90, | (0.72, | (1.12, |
| | 7.64) | 8.49) | 4.25) | 4.29) |
| Adverse events leading to discontinuation of study agent | 7.46 | 4.97 | 4.23 | 4.44 |
| | (3.58, | (1.83, | (2.25, | (2.67, |
| | 13.72) | 10.83) | 7.23) | 6.93) |
| Death | 0.00 | 0.00 | 0.33 | 0.23 |
| | (0.00, | (0.00, | (0.01, | (0.01, |
| | 2.24) | 2.48) | 1.81) | 1.30) |
| All malignancies | 1.49 | 0.83 | 0.98 | 0.93 |
| | (0.18, | (0.02, | (0.20, | (0.25, |
| | 5.39) | 4.62) | 2.85) | 2.39) |
| Excluding nonmelanoma skin cancer | 0.75 | 0.00 | 0.00 | 0.00 |
| | (0.02, | (0.00, | (0.00, | (0.00, |
| | 4.16) | 2.48) | 0.97) | 0.70) |
| Nonmelanoma skin cancer | 0.75 | 0.83 | 0.98 | 0.93 |
| | (0.02, | (0.02, | (0.20, | (0.25, |
| | 4.16) | 4.62) | 2.85) | 2.39) |

Abbreviations: CI = confidence interval; IV = intravenous; q8w = every 8 weeks; q12w = every 12 weeks; SC = subcutaneous; UC = ulcerative colitis
^{a} Includes 1) data from Week 44 through Week 96, or up to the dose adjustment if subjects had a dose adjustment during the long-term extension, for subjects who were in clinical response to ustekinumab IV induction dosing and were randomized to placebo SC on entry into the maintenance study; and 2) data from Week 44 through Week 96 for subjects who were in clinical response to placebo IV induction dosing and received placebo SC on entry into the maintenance study.
^{b} Includes data from Week 44 through Week 96, or up to the dose adjustment if subjects had a dose adjustment during the long-term extension for subjects who were in clinical response to ustekinumab IV induction dosing and were randomized to ustekinumab 90 mg SC q12w on entry into the maintenance study.
c Includes: 1) Subjects who were in clinical response to ustekinumab IV induction dosing and were randomized to receive ustekinumab 90 mg SC q8w on entry into the maintenance study, with data from Week 44 through Week 96; 2) Subjects who were in clinical response to ustekinumab IV induction dosing, randomized to receive placebo SC or ustekinumab 90 mg SC q12w on entry into the maintenance study, and had a dose adjustment to ustekinumab 90 mg SC q8w, with data from the time of dose adjustment onward; 3) Subjects who were not in clinical response to ustekinumab at induction Week 8 but were in clinical response at induction Week 16 after a SC administration of ustekinumab at induction Week 8 and received ustekinumab 90 mg SC q8w on entry into the maintenance study with data from Week 44 through Week 96
^{d} Confidence intervals based on an exact method assuming that the observed number of events follows a Poisson distribution.
^{e} Infection as assessed by the investigator.

The CNTO1275UCO3001 maintenance study through 44 weeks of treatment provided consistent and convincing evidence that ustekinumab 90 mg SC q12w or q8w dose regimens were both effective in subjects with moderate to severe UC who had responded to a single ustekinumab IV induction dose. Namely, ustekinumab maintenance treatment sustained clinical response and clinical remission and resulted in corticosteroid-free clinical remission and endoscopic healing.
The objectives of this portion of the study LTE were to assess the efficacy, PK, immunogenicity, and safety of an additional 1 year of treatment with ustekinumab in subjects with moderately to severely active UC who had completed the 44-week maintenance study and entered the study LTE.
During the study LTE, subjects received the same dose regimen they were receiving at Week 44 of the maintenance study. The first study agent administration in the study LTE occurred at Week 48. Subjects who were in clinical response to a single IV induction dose of ustekinumab and were randomized at Week 0 of the maintenance study were the primary population for the maintenance study; these subjects were eligible for dose adjustment beginning at Week 56 of the LTE; subjects randomized to placebo could receive ustekinumab q8w, those randomized to ustekinumab q12w could receive ustekinumab q8w, and those randomized to ustekinumab q8w had a sham dose adjustment prior to study unblinding. Subjects who were not randomized in the maintenance study were not eligible for a dose adjustment; these subjects included those who were delayed responders to ustekinumab IV induction and received 90 mg SC ustekinumab q8w during the LTE, as well as placebo IV induction responders who remained on placebo.
The study blind was maintained in the study LTE until the last subject in the main study completed the Week 44 evaluations and the Week 44 analyses were completed. Subjects continued to receive study agent at monthly visits until that time. After the study was unblinded to the investigative sites, subjects receiving placebo were terminated from study participation, and subjects receiving ustekinumab continued to receive ustekinumab and had study visits scheduled to coincide with their dose regimen (either every 8 or 12 weeks, as appropriate to their dose regimen).
Of the 523 randomized subjects who participated in the maintenance study, 399 (76.3%) subjects continued treatment in the study LTE including 284 subjects who continued to receive ustekinumab. This included 141 subjects receiving ustekinumab 90 mg SC q12w and 143 subjects receiving ustekinumab 90 mg SC q8w. Among these randomized, ustekinumab-treated subjects, rates of discontinuation were low with 276 of the 284 (97.2%) subjects completing study participation through Week 96. Additionally, among all randomized subjects and nonrandomized subjects in the ustekinumab delayed-responder group receiving ustekinumab during the study LTE, 388/400 (95.3%) subjects completed study participation through Week 96. During the study LTE through Week 96, the placebo groups had 12 fewer weeks of follow-up on average (37.1 weeks) as compared to the combined ustekinumab groups (49.1 weeks), which is primarily attributed to the protocol-specified discontinuation of placebo subjects at the time of study unblinding.
Overall, the baseline demographics at Week 0 of induction were similar among randomized and nonrandomized subjects treated in the study LTE. Among randomized subjects, the majority of subjects were male with a median age of 40 years and a median body weight of 71.6 kg. In general, the induction baseline clinical disease characteristics of randomized subjects who were treated in the LTE are representative of a population with moderately to severely active UC and are similar to the disease characteristics of the randomized population who entered at Week 0 of the maintenance study. The UC disease characteristics at Week 0 of induction and Week 0 of maintenance for randomized and nonrandomized subjects who were treated in the LTE were consistent with those of all randomized and nonrandomized subjects who entered the maintenance study. At Week 44 of maintenance, measures of UC disease activity (eg, Mayo and IBDQ scores) were generally comparable among subjects randomized to ustekinumab q12w and q8w with 46.1% and 52.4% in clinical remission and 56.7% and 61.5% with endoscopic healing, respectively. Among the nonrandomized delayed responders treated in the LTE, disease activity measures indicated benefit from ustekinumab maintenance therapy; however, across measures these subjects tended to have somewhat higher disease activity at Week 44 of maintenance and inflammatory burden accompanied by lower rates of clinical remission (38.8%) and endoscopic healing (47.4%) relative to those subjects in response to a single induction dose of IV ustekinumab and randomized to ustekinumab q8w.
There were important limitations of the design of the study LTE that are worth noting. First, subjects were selected by the investigator to participate in the study LTE because, in their opinion, they might benefit from continued treatment. This criterion may limit the generalizability of the findings to only those who responded to and tolerated ustekinumab in the first year of treatment. Second, subjects could change concomitant medications at any time during the LTE to mimic real world practice. Third, direct efficacy comparisons between placebo and ustekinumab treatment groups were not performed since subjects who entered the study LTE on placebo represent a group of patients who were long-term responders to ustekinumab induction or were true placebo responders. Importantly, placebo subjects were discontinued from the study when study unblinding occurred limiting the value of direct comparisons between the placebo and ustekinumab treatment groups. As a result, the emphasis of clinical outcomes reported herein is on efficacy measures among ustekinumab-treated subjects. It should be recognized that the main intent of presenting data for each of the ustekinumab 90 mg q12w and q8w treatment groups is to show that both of these treatment regimens maintained remission over time; as the study was not designed to compare between ustekinumab groups, results are descriptive, and any comparisons should be interpreted with caution. In addition, when considering the data for dose adjustment within the randomized population, it should be noted that the decision to dose adjust was based on the clinical judgement of the investigator regarding a subject's disease activity; no protocol-specified criteria (e.g., clinical flare based on partial Mayo score) were applied and some subjects were in remission at the time of the dose adjustment, thereby limiting the interpretability of these data. Finally, clinical outcomes in subpopulations based on biologic-failure status (i.e., biologic naïve, biologic nonfailure, and biologic failure) are presented for the purpose of evaluating the consistency of outcomes in these populations with those in the overall population; however, due to the limited sample sizes in these subpopulations, these results should also be interpreted with caution.
With the above caveats noted, among randomized subjects continuing treatment in the study LTE, ustekinumab 90 mg SC q12w and 90 mg SC q8w were effective in maintaining remission measured as either symptomatic or partial Mayo remission through the second year of treatment. When dose adjustment was considered as a treatment strategy for ustekinumab (and not a treatment failure), rates of symptomatic remission or partial Mayo remission were also maintained over time. Through Week 92 with dose adjustment considered as treatment failure, reductions in mean daily P.Eq. corticosteroid dose were generally sustained; the majority (93%) of subjects who were receiving corticosteroids at maintenance baseline were not receiving corticosteroids at Week 92. Of note, among subjects who were in symptomatic remission at Week 92, the majority (99%) were corticosteroid-free. Similar results were observed when remission was measured using the partial Mayo score. Furthermore, reductions in inflammatory burden (ie, CRP as well as fecal calprotectin and lactoferrin) at maintenance baseline were sustained through 2 years of ustekinumab treatment. In addition, improvements in health-related quality of life, measured by IBDQ and SF-36, that were measured at maintenance baseline were generally maintained with continued ustekinumab treatment in the study LTE. Sustained efficacy was observed regardless of biologic-failure status including subjects with a history of biologic failure as well as those subjects who did not have a history of biologic failure or were naïve to biologic therapy. Furthermore, some clinical benefit was observed for subjects whose disease activity worsened in the opinion of the investigator and had a dose adjustment from ustekinumab q12w to q8w.
Importantly, among nonrandomized subjects who were delayed responders to ustekinumab IV induction, similar results indicating a sustained benefit of ustekinumab maintenance treatment in this group of subjects were also observed.
Continued maintenance therapy with ustekinumab 90 mg SC q12w or q8w resulted in sustained ustekinumab exposure during the study LTE that was comparable to ustekinumab levels observed through Week 44 of the maintenance phase. Dose adjustment to ustekinumab q8w occurred at different visits thus concentration data summaries for subjects who had a dose adjustment were not representative of the expected concentrations at the respective timepoints for those on ustekinumab q8w. No clear exposure-response relationship was observed between serum ustekinumab concentration and symptomatic or partial remission at Week 92 in randomized subjects who were considered to have benefited from maintenance treatment and were treated in the study LTE.
Overall, the proportion of subjects who were positive for antibodies to ustekinumab was low, with 22 of 400 (5.5%) randomized subjects who received ustekinumab during induction and maintenance and continued to receive ustekinumab during the study LTE positive for antibodies at any time through Week 96.
In the study LTE through Week 96, ustekinumab was generally well tolerated, with a safety profile consistent with the well-described overall ustekinumab safety profile and similar to the safety observations in the first year of the maintenance study. There were no new safety signals identified.
Among all treated subjects, the number of events per hundred subject-years of AEs, SAEs, and serious infections were generally comparable among the q12w and q8w groups individually and among combined ustekinumab groups compared to placebo with no evidence of a dose effect. Similar to the findings through Week 44, the Infections and infestations SOC and Gastrointestinal disorders SOC had the highest incidence of AE's from Week 44 through Week 96. Nasopharyngitis was the most frequently reported infection in all treatment groups occurring in 19.40, 29.01, and 27.95 per hundred subject years in the placebo, q12w, and q8w groups, respectively. Ulcerative colitis was the most frequently reported AE in the Gastrointestinal disorders SOC reported in 42.54, 15.75, and 18.53 per hundred subject years in the placebo, q12w and q8w groups, respectively. The number of subjects reporting SAEs was comparable across treatment groups with UC being the SAE of highest incidence in both the placebo and combined ustekinumab groups (5.22 and 1.63 per hundred subject-years, respectively). One death due to cardiac arrest was reported in a subject randomized to the placebo group at maintenance baseline and had a dose adjustment during the study LTE, receiving a single ustekinumab dose for worsening UC with concurrent CMV colitis. In addition to this subject who was categorized as having a serious MACE, 2 subjects reported myocardial infarction, and 1 subject reported a nonserious event of retinal vein occlusion.
Serious infections were infrequent events and no event was reported in more than 1 subject. No cases of TB were reported in ustekinumab-treated subjects. Two subjects were reported with serious infections considered to be opportunistic infections: one report of CMV colitis described above, and an event of L. monocytogenes reported for a subject in the ustekinumab 90 mg q8w group.
Rates of injection-site reactions remained low from Weeks 44 to 96, with no reports of serious reactions, anaphylaxis to ustekinumab, or serum sickness-like reactions. Similar to the findings through the first year of the study, injection-site erythema was the most commonly occurring reaction. No relationship between the development of antibodies to ustekinumab and injection-site reactions was identified in this study.
Among subjects treated with ustekinumab in the study LTE, 3 subjects were reported to have NMSC: 2 subjects (1 subject each from the ustekinumab 90 mg q12w group and ustekinumab delayed-responder group [receiving ustekinumab 90 mg q8w]) with BCC and 1 subject with SCC and BCC (ustekinumab delayed-responder group [receiving ustekinumab 90 mg q8w]). Age, prior immunomodulator use, and sun exposure were confounding factors. One additional subject randomized to placebo (i.e., exposed to IV ustekinumab in induction) also was reported with BCC. One subject randomized to placebo also reported lentigo malignant melanoma.
Overall, the safety profile for ustekinumab among the delayed induction responders receiving ustekinumab 90 mg q8w as well as for those randomized subjects who had a dose adjustment to ustekinumab 90 mg q8w was consistent with that reported in the subjects randomized to ustekinumab 90 mg q8w; no new safety signals were identified in either group.
In summary, subjects with moderately to severely active UC who received ustekinumab in the study LTE, sustained symptomatic and partial Mayo remission through the second year of exposure to the drug. Remission was achieved in the absence of corticosteroids for the majority of patients. Clinical outcomes were supported by sustained reductions in inflammatory markers and improvements in health-related quality of life outcome measures. The safety profile observed for ustekinumab in the second year of treatment was consistent with the safety through the first year of treatment and with the established ustekinumab safety profile with no new safety signals identified.

### Conclusions

Treatment with ustekinumab 90 mg SC q12w and q8w maintained remission measured as either symptomatic remission or partial Mayo remission through the second year of treatment.
Maintenance of efficacy through a second year of treatment was supported by sustained reductions in inflammatory markers of disease and sustained improvement in health-related quality of life measures.
No new safety signals were identified in the second year of maintenance therapy.
   The safety profile is consistent with previously reported safety data through the first year of treatment in UC and with the overall ustekinumab safety profile.

What is claimed:

1. A method of treating moderately to severely active ulcerative colitis (UC) in a subject in need thereof, comprising administering to the subject an anti-IL-12/IL-23p40 antibody, wherein the antibody comprises a heavy chain variable region and a light chain variable region, the heavy chain variable region comprising: a heavy chain complementarity determining region 1 (CDRH1) amino acid sequence of SEQ ID NO:1; a heavy chain CDRH2 amino acid sequence of SEQ ID NO:2; and a heavy chain CDRH3 amino acid sequence of SEQ ID NO:3; and the light chain variable region comprising: a light chain complementarity determining region 1 (CDRL1) amino acid sequence of SEQ ID NO:4; a light chain CDRL2 amino acid sequence of SEQ ID NO:5; and a light chain CDRL3 amino acid sequence of SEQ ID NO:6, and wherein the subject is a responder to treatment at week 92 of treatment based on satisfying one or more clinical endpoints selected from the group consisting of:
(a) symptomatic remission;
(b) partial Mayo remission;
(c) Mayo rectal bleeding subscore of 0;
(d) Mayo stool frequency subscore of 0 or 1;
(e) mean absolute stool numbers decreased by at least 3;
(f) decrease in corticosteroid usage and/or dosage;
(g) corticosteroid-free symptomatic remission;
(h) corticosteroid-free partial mayo remission;
(i) normalized fecal lactoferrin;
(j) normalization of fecal calprotectin levels;
(k) ≥16-point improvement from induction baseline in the total Inflammatory Bowel Disease Questionnaire (IBDQ) score;
(l) IBDQ remission;
(m) a ≥5-point improvement from induction baseline in the SF-36 PCS score; and
(n) a ≥5-point improvement from induction baseline in the SF-36 MCS score.

2. The method of claim 1, wherein the antibody comprises a heavy chain variable region comprising the amino acid sequence of SEQ ID NO:7 and a light chain variable region comprising the amino acid sequence of SEQ ID NO: 8.

3. The method of claim 1, wherein the antibody comprises a heavy chain amino acid sequence of SEQ ID NO:10 and a light chain amino acid sequence of SEQ ID NO:11.


SEQUENCE LISTING (11 sequences)

SEQ ID NO: 1 - protein, 5 aa, synthetic construct
  REGION 1..5: note = anti-IL-12/IL-23p40 antibody complementarity determining region heavy chain 1
TYWLG

SEQ ID NO: 2 - protein, 17 aa, synthetic construct
  REGION 1..17: note = anti-IL-12/IL-23p40 antibody complementarity determining region heavy chain 2
IMSPVDSDIRYSPSFQG

SEQ ID NO: 3 - protein, 10 aa, synthetic construct
  REGION 1..10: note = anti-IL-12/IL-23p40 antibody complementarity determining region heavy chain 3
RRPGQGYFDF

SEQ ID NO: 4 - protein, 11 aa, synthetic construct
  REGION 1..11: note = anti-IL-12/IL-23p40 antibody complementarity determining region light chain 1
RASQGISSWLA

SEQ ID NO: 5 - protein, 7 aa, synthetic construct
  REGION 1..7: note = anti-IL-12/IL-23p40 antibody complementarity determining region light chain 2
AASSLQS

SEQ ID NO: 6 - protein, 9 aa, synthetic construct
  REGION 1..9: note = anti-IL-12/IL-23p40 antibody complementarity determining region light chain 3
QQYNIYPYT

SEQ ID NO: 7 - protein, 119 aa, synthetic construct
  REGION 1..119: note = anti-IL-12/IL-23p40 antibody variable heavy chain region
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 8 - protein, 108 aa, synthetic construct
  REGION 1..108: note = anti-IL-12/IL-23p40 antibody variable light chain region
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKR

SEQ ID NO: 9 - protein, 503 aa, synthetic construct
  REGION 1..503: note = Human IL-12 with alpha and beta subunits
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 10 - protein, 449 aa, synthetic construct
  REGION 1..449: note = anti-IL-12/IL-23p40 antibody heavy chain
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 214 aa, synthetic construct
  REGION 1..214: note = anti-IL-12/IL-23p40 antibody light chain
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC